(19) United States
(12) Patent Application Publication
(10) Pub. No.: US 2024/0110180 A1
(43) Pub. Date: Apr. 4, 2024

(54) COMPOSITIONS AND METHODS FOR INHIBITION OF EXPRESSION OF APOLIPOPROTEIN C-III (APOC3) GENES

(71) Applicant: Alnylam Pharmaceuticals, Inc., Cambridge, MA (US)

(72) Inventors: Brian Bettencourt, Groton, MA (US); Kevin Fitzgerald, Brookline, MA (US); Stuart Milstein, Arlington, MA (US); Martin A. Maier, Belmont, MA (US); Klaus Charisse, Acton, MA (US); Kallanthottathil G. Rajeev, Wayland, MA (US); Satyanarayana Kuchimanchi, Acton, MA (US); Muthiah Manoharan, Weston, MA (US); Tuyen M. Nguyen, Milton, MA (US)

(21) Appl. No.: 18/199,390

(22) Filed: May 19, 2023

Related U.S. Application Data
(63) Continuation of application No. 17/356,946, filed on Jun. 24, 2021, now abandoned.
Application No. 17/356,946 is a continuation of application No. 16/700,870, filed on Dec. 2, 2019, now abandoned.
Application No. 16/700,870 is a continuation of application No. 16/383,273, filed on Apr. 12, 2019, now abandoned.
Application No. 16/383,273 is a continuation of application No. 15/948,624, filed on Apr. 9, 2018, now abandoned.
Application No. 15/948,624 is a continuation of application No. 15/076,269, filed on Mar. 21, 2016, now Pat. No. 9,970,006.
Application No. 15/076,269 is a continuation of application No. 14/125,567, filed on Dec. 11, 2013, now Pat. No. 9,315,813.
(60) Provisional application No. 61/499,620, filed on Jun. 21, 2011.

(51) Int. Cl. C12N 15/113

(52) U.S. Cl. CPC C12N 15/113; C12N 15/1137; C12N 2310/14; C12N 2310/315; C12N 2310/321; C12N 2310/344; C12N 2310/3515; C12N 2310/3521

(57) ABSTRACT

The invention relates to double-stranded ribonucleic acid (dsRNA) targeting an APOC3 gene, and methods of using the dsRNA to inhibit expression of APOC3.

### CROSS REFERENCE TO RELATED APPLICATIONS

This application is a continuation of U.S. patent application Ser. No. 17/356,946, filed on Jun. 24, 2021, which is a continuation of U.S. patent application Ser. No. 16/700,870, filed on Dec. 2, 2019, abandoned, which is a continuation of U.S. application Ser. No. 16/383,273, filed on Apr. 12, 2019, abandoned, which is a continuation of U.S. application Ser. No. 15/948,624, filed on Apr. 9, 2018, abandoned, which is a continuation of U.S. application Ser. No. 15/076,269, filed on Mar. 21, 2016, now U.S. Pat. No. 9,970,006, issued on May 15, 2018, which is a continuation of U.S. application Ser. No. 14/125,567, filed on Dec. 11, 2013, now U.S. Pat. No. 9,315,813, issued on Apr. 19, 2016, which is the National Phase of International Application No. PCT/US2012/043642, filed on Jun. 21, 2012, which claims the benefit of priority to U.S. Provisional Application No. 61/499,620, filed on Jun. 21, 2011. The entire contents of each of the foregoing applications is incorporated herein by reference.

### SEQUENCE LISTING

The application contains a Sequence Listing which has been submitted electronically in .XML format and is hereby incorporated by reference in its entirety. Said .XML copy, created on Oct. 10, 2023, is named “121301_15007_SL.xml” and is 7,359,147 bytes in size. The sequence listing contained in this .XML file is part of the specification and is hereby incorporated by reference herein in its entirety.

### FIELD OF THE INVENTION

The invention relates to double-stranded ribonucleic acid (dsRNA) targeting an APOC3 gene, and methods of using the dsRNA to inhibit expression of APOC3.

### BACKGROUND OF THE INVENTION

In the U.S., 30% of adults have elevated triglycerides (TG) >150 mg/dL. The prevalence of adults with severe hypertriglyceridemia (TG>500 mg/dL) is 1.7%. Current treatments include lifestyle modification (diet, exercise and smoking cessation), prescription grade fish oil, fibrates, and niacin.

ApoC3 is a secreted liver protein shown to inhibit lipoprotein lipases that hydrolyze TG into free fatty acids; inhibit ApoE-mediated hepatic uptake of TG-rich lipoproteins through LDLR and LRP as well as receptor independent endocytosis; and promote hepatic VLDL secretion. At least one mutation in the human APOC3 gene has been associated with a favorable lipid profile. (Pollin T I et al. (2008) A null mutation in human APOC3 confers a favorable plasma lipid profile and apparent cardioprotection. Science. 322(5908):1702-5).

Double-stranded RNA molecules (dsRNA) have been shown to block gene expression in a highly conserved regulatory mechanism known as RNA interference (RNAi). WO 99/32619 (Fire et al.) discloses the use of a dsRNA of at least 25 nucleotides in length to inhibit the expression of genes in C. elegans. dsRNA has also been shown to degrade target RNA in other organisms, including plants (see, e.g., WO 99/53050, Waterhouse et al.; and WO 99/61631, Heifetz et al.), Drosophila (see, e.g., Yang, D., et al., Curr. Biol. (2000) 10:1191-1200), and mammals (see WO 00/44895, Limmer; and DE 101 00 586.5, Kreutzer et al.).

### SUMMARY OF THE INVENTION

Disclosed herein are double-stranded ribonucleic acid (dsRNA) for inhibiting expression of an APOC3 gene, wherein the dsRNA comprises a sense strand and an antisense strand each 30 nucleotides or less in length, wherein the antisense strand comprises at least 15 contiguous nucleotides of an antisense sequence in Table 1, 2, 6, 7, or 10. In one embodiment, the dsRNA comprises a sense strand consisting of the nucleotide sequence SEQ ID NO:70 and an antisense strand consisting of a nucleotide sequence SEQ ID NO: 151 (AD-45149.1 UM). In another embodiment, the sense strand sequence is selected from Table 1, 2, 6, 7, or 10, and the antisense strand is selected from Table 1, 2, 6, 7, or 10.

In some embodiments, at least one nucleotide of the dsRNA is a modified nucleotide, e.g., at least one modified nucleotide is chosen from the group consisting of: a 2′-O-methyl modified nucleotide, a nucleotide comprising a 5′-phosphorothioate group, and a terminal nucleotide linked to a cholesteryl derivative or dodecanoic acid bisdecylamide group, a 2′-deoxy-2′-fluoro modified nucleotide, a 2′-deoxy-modified nucleotide, a locked nucleotide, an abasic nucleotide, 2′-amino-modified nucleotide, 2′-alkyl-modified nucleotide, morpholino nucleotide, a phosphoramidate, and a non-natural base comprising nucleotide.

In some embodiments, at least one strand comprises a 3′ overhang of at least 1 nucleotide or each strand comprises a 3′ overhang of at 2 nucleotides.

Any dsRNA of the invention can further comprising a ligand, for example a ligand that is conjugated to the 3′ end of the sense strand of the dsRNA. In some embodiments, a dsRNA of the invention further comprises at least one N-Acetyl-Galactosamine.

In addition the invention provides a cell comprising any dsRNA of the invention; a vector encoding at least one strand of any dsRNA of the invention and a cell comprising the vector.

Also included in the invention are pharmaceutical compositions for inhibiting expression of an APOC3 gene comprising any dsRNA of the invention. The pharmaceutical composition can include a lipid formulation, e.g., a lipid formulation comprising MC3.

Another aspect of the invention is a method of inhibiting APOC3 expression in a cell, the method comprising: (a) contacting the cell a APOC3 dsRNA of the invention and (b) maintaining the cell produced in step (a) for a time sufficient to obtain degradation of the mRNA transcript of an APOC3 gene, thereby inhibiting expression of the APOC3 gene in the cell. In some embodiments APOC3 expression is inhibited by at least 30%.

A further aspect of the invention is a method of treating a disorder mediated by APOC3 expression comprising administering to a human in need of such treatment a therapeutically effective amount of the APOC3 dsRNA of the invention or a pharmaceutical composition of the invention. The disorder can be, e.g., elevated triglyceride levels, e.g., triglyceride levels >150 mg/dL or >500 mg/dL. In some embodiments administration causes an increase in lipoprotein lipase and/or hepatic lipase activity. The dsRNA or the pharmaceutical composition can be administered at a dose of about 0.01 mg/kg to about 10 mg/kg or about 0.5 mg/kg to about 50 mg/kg.

### DESCRIPTION OF THE DRAWINGS

FIG. 1 is a graph showing the effect on target mRNA, triglyceride (TG) and total cholesterol levels in mice after treatment with siRNA targeting APOC3 (“siRNA #1” and siRNA #2”).

FIG. 2 shows the structure of GalNAc.

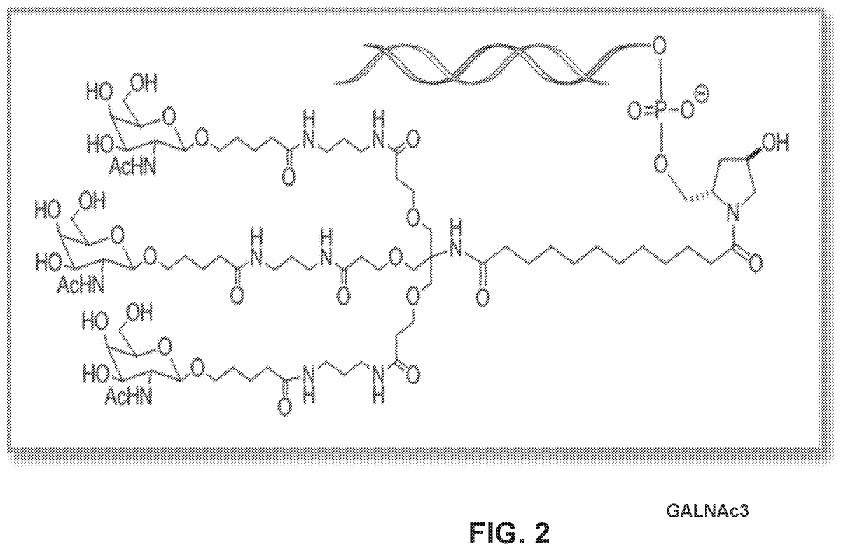

FIG. 3 shows the structure of an siRNA conjugated to Chol-p-(GalNAc)₃ via phosphate linkage at the 3′ end.

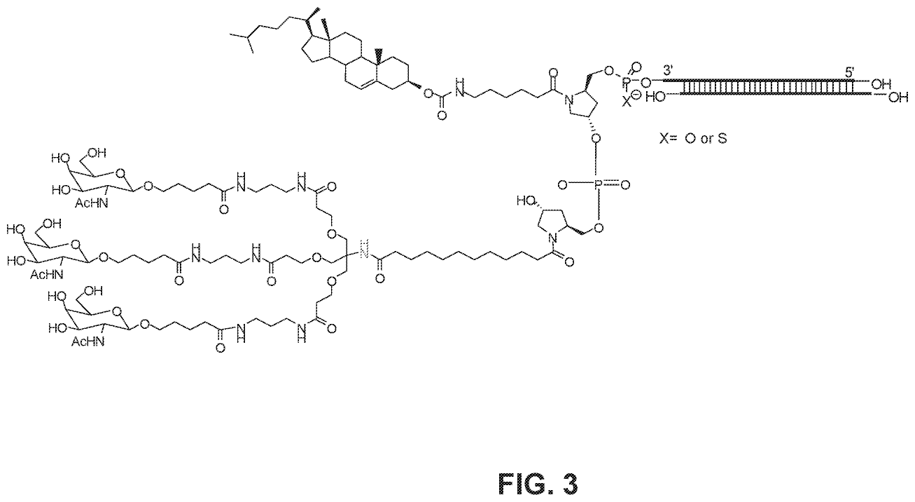

FIG. 4 shows the structure of an siRNA conjugated to LCO(GalNAc)₃ (a (GalNAc)₃-3′-Lithocholic-oleoyl siRNA Conjugate).

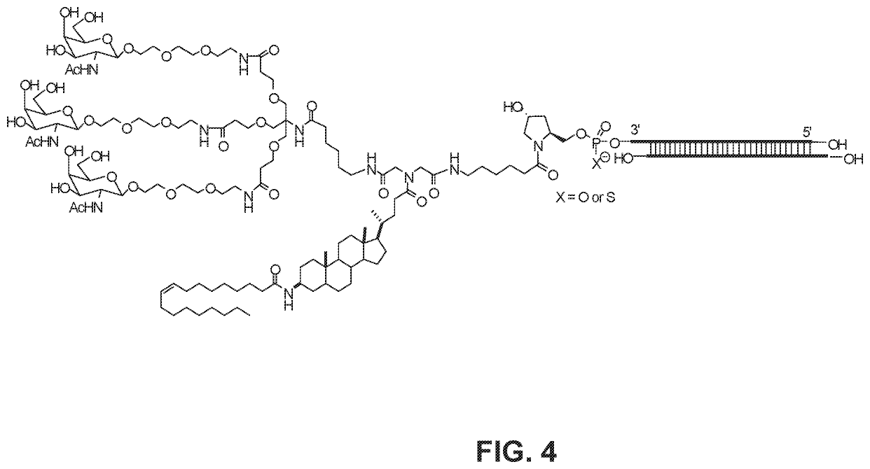

### DETAILED DESCRIPTION OF THE INVENTION

The details of one or more embodiments of the invention are set forth in the description below. Other features, objects, and advantages of the invention will be apparent from the description and the drawings, and from the claims.

The invention provides dsRNAs and methods of using the dsRNAs for inhibiting the expression of an APOC3 gene in a cell or a mammal where the dsRNA targets an APOC3 gene. The invention also provides compositions and methods for treating pathological conditions and diseases in a mammal caused by the expression of an APOC3 gene. AN APOC3 dsRNA directs the sequence-specific degradation of APOC3 mRNA.

### Definitions

For convenience, the meaning of certain terms and phrases used in the specification, examples, and appended claims, are provided below. If there is an apparent discrepancy between the usage of a term in other parts of this specification and its definition provided in this section, the definition in this section shall prevail.

“G,” “C,” “A” and “U” each generally stand for a nucleotide that contains guanine, cytosine, adenine, and uracil as a base, respectively. “T” and “dT” are used interchangeably herein and refer to a deoxyribonucleotide wherein the nucleobase is thymine, e.g., deoxyribothymine. However, it will be understood that the term “ribonucleotide” or “nucleotide” or “deoxyribonucleotide” can also refer to a modified nucleotide, as further detailed below, or a surrogate replacement moiety. The skilled person is well aware that guanine, cytosine, adenine, and uracil may be replaced by other moieties without substantially altering the base pairing properties of an oligonucleotide comprising a nucleotide bearing such replacement moiety. For example, without limitation, a nucleotide comprising inosine as its base may base pair with nucleotides containing adenine, cytosine, or uracil. Hence, nucleotides containing uracil, guanine, or adenine may be replaced in the nucleotide sequences of the invention by a nucleotide containing, for example, inosine. Sequences comprising such replacement moieties are embodiments of the invention.

“APOC3” refers to the Apolipoprotein C-III gene. According to the NCBI NLM website, Apolipoprotein C-III is a very low density lipoprotein (VLDL) protein. APOC3 inhibits lipoprotein lipase and hepatic lipase; it is thought to delay catabolism of triglyceride-rich particles. The APOA1, APOC3 and APOA4 genes are closely linked in both rat and human genomes. The A-I and A-IV genes are transcribed from the same strand, while the A-1 and C-III genes are convergently transcribed. An increase in apoC-III levels induces the development of hypertriglyceridemia. A human APOC3 mRNA sequence is GenBank accession number NM_000040.1, included herein as SEQ ID NO:1. A cynomolgus monkey (Macaca fascicularis) ANGPTL3 mRNA sequence is GenBank accession number X68359.1.

As used herein, “target sequence” refers to a contiguous portion of the nucleotide sequence of an mRNA molecule formed during the transcription of an APOC3 gene, including mRNA that is a product of RNA processing of a primary transcription product.

As used herein, the term “strand comprising a sequence” refers to an oligonucleotide comprising a chain of nucleotides that is described by the sequence referred to using the standard nucleotide nomenclature.

As used herein, and unless otherwise indicated, the term “complementary,” when used to describe a first nucleotide sequence in relation to a second nucleotide sequence, refers to the ability of an oligonucleotide or polynucleotide comprising the first nucleotide sequence to hybridize and form a duplex structure under certain conditions with an oligonucleotide or polynucleotide comprising the second nucleotide sequence, as will be understood by the skilled person.

For example, a first nucleotide sequence can be described as complementary to a second nucleotide sequence when the two sequences hybridize (e.g., anneal) under stringent hybridization conditions. Hybridization conditions include temperature, ionic strength, pH, and organic solvent concentration for the annealing and/or washing steps. The term stringent hybridization conditions refers to conditions under which a first nucleotide sequence will hybridize preferentially to its target sequence, e.g., a second nucleotide sequence, and to a lesser extent to, or not at all to, other sequences. Stringent hybridization conditions are sequence dependent, and are different under different environmental parameters. Generally, stringent hybridization conditions are selected to be about 5° C. lower than the thermal melting point (Tₘ) for the nucleotide sequence at a defined ionic strength and pH. The Tₘ is the temperature (under defined ionic strength and pH) at which 50% of the first nucleotide sequences hybridize to a perfectly matched target sequence. An extensive guide to the hybridization of nucleic acids is found in, e.g., Tijssen (1993) Laboratory Techniques in Biochemistry and Molecular Biology-Hybridization with Nucleic Acid Probes part I, chap. 2, “Overview of principles of hybridization and the strategy of nucleic acid probe assays,” Elsevier, N.Y. (“Tijssen”).

Other conditions, such as physiologically relevant conditions as may be encountered inside an organism, can apply. The skilled person will be able to determine the set of conditions most appropriate for a test of complementarity of two sequences in accordance with the ultimate application of the hybridized nucleotides.

This includes base-pairing of the oligonucleotide or polynucleotide comprising the first nucleotide sequence to the oligonucleotide or polynucleotide comprising the second nucleotide sequence over the entire length of the first and second nucleotide sequence. Such sequences can be referred to as “fully complementary” with respect to each other herein. However, where a first sequence is referred to as “substantially complementary” with respect to a second sequence herein, the two sequences can be fully complementary, or they may form one or more, but generally not more than 4, 3 or 2 mismatched base pairs upon hybridization, while retaining the ability to hybridize under the conditions most relevant to their ultimate application. However, where two oligonucleotides are designed to form, upon hybridization, one or more single stranded overhangs, such overhangs shall not be regarded as mismatches with regard to the determination of complementarity. For example, a dsRNA comprising one oligonucleotide 21 nucleotides in length and another oligonucleotide 23 nucleotides in length, wherein the longer oligonucleotide comprises a sequence of 21 nucleotides that is fully complementary to the shorter oligonucleotide, may yet be referred to as “fully complementary” for the purposes described herein.

“Complementary” sequences, as used herein, may also include, or be formed entirely from, non-Watson-Crick base pairs and/or base pairs formed from non-natural and modified nucleotides, in as far as the above requirements with respect to their ability to hybridize are fulfilled. Such non-Watson-Crick base pairs includes, but not limited to, G:U Wobble or Hoogstein base pairing.

The terms “complementary,” “fully complementary” and “substantially complementary” herein may be used with respect to the base matching between the sense strand and the antisense strand of a dsRNA, or between the antisense strand of a dsRNA and a target sequence, as will be understood from the context of their use.

As used herein, a polynucleotide that is “substantially complementary to at least part of” a messenger RNA (mRNA) refers to a polynucleotide that is substantially complementary to a contiguous portion of the mRNA of interest (e.g., an mRNA encoding APOC3) including a 5′ UTR, an open reading frame (ORF), or a 3′ UTR. For example, a polynucleotide is complementary to at least a part of an APOC3 mRNA if the sequence is substantially complementary to a non-interrupted portion of an mRNA encoding APOC3.

In one embodiment, the antisense strand of the dsRNA is sufficiently complementary to a target mRNA so as to cause cleavage of the target mRNA.

The term “double-stranded RNA” or “dsRNA,” as used herein, refers to a complex of ribonucleic acid molecules, having a duplex structure comprising two anti-parallel and substantially complementary, as defined above, nucleic acid strands. In general, the majority of nucleotides of each strand are ribonucleotides, but as described in detail herein, each or both strands can also include at least one non-ribonucleotide, e.g., a deoxyribonucleotide and/or a modified nucleotide. In addition, as used in this specification, “dsRNA” may include chemical modifications to ribonucleotides, including substantial modifications at multiple nucleotides and including all types of modifications disclosed herein or known in the art. Any such modifications, as used in an siRNA type molecule, are encompassed by “dsRNA” for the purposes of this specification and claims.

The two strands forming the duplex structure may be different portions of one larger RNA molecule, or they may be separate RNA molecules. Where the two strands are part of one larger molecule, and therefore are connected by an uninterrupted chain of nucleotides between the 3′-end of one strand and the 5′-end of the respective other strand forming the duplex structure, the connecting RNA chain is referred to as a “hairpin loop.” Where the two strands are connected covalently by means other than an uninterrupted chain of nucleotides between the 3′-end of one strand and the 5′-end of the respective other strand forming the duplex structure, the connecting structure is referred to as a “linker.” The RNA strands may have the same or a different number of nucleotides. The maximum number of base pairs is the number of nucleotides in the shortest strand of the dsRNA minus any overhangs that are present in the duplex. In addition to the duplex structure, a dsRNA may comprise one or more nucleotide overhangs. The term “siRNA” is also used herein to refer to a dsRNA as described above.

As used herein, a “nucleotide overhang” refers to the unpaired nucleotide or nucleotides that protrude from the duplex structure of a dsRNA when a 3′-end of one strand of the dsRNA extends beyond the 5′-end of the other strand, or vice versa. “Blunt” or “blunt end” means that there are no unpaired nucleotides at that end of the dsRNA, i.e., no nucleotide overhang. A “blunt ended” dsRNA is a dsRNA that is double-stranded over its entire length, i.e., no nucleotide overhang at either end of the molecule.

The term “antisense strand” refers to the strand of a dsRNA which includes a region that is substantially complementary to a target sequence. As used herein, the term “region of complementarity” refers to the region on the antisense strand that is substantially complementary to a sequence, for example a target sequence, as defined herein. Where the region of complementarity is not fully complementary to the target sequence, the mismatches are most tolerated in the terminal regions and, if present, are generally in a terminal region or regions, e.g., within 6, 5, 4, 3, or 2 nucleotides of the 5′ and/or 3′ terminus.

The term “sense strand,” as used herein, refers to the strand of a dsRNA that includes a region that is substantially complementary to a region of the antisense strand.

As used herein, the term “nucleic acid lipid particle” includes the term “SNALP” and refers to a vesicle of lipids coating a reduced aqueous interior comprising a nucleic acid such as a dsRNA or a plasmid from which a dsRNA is transcribed. Nucleic acid lipid particles, e.g., SNALP are described, e.g., in U.S. Patent Application Publication Nos. 20060240093, 20070135372, and U.S. Ser. No. 61/045,228 filed on Apr. 15, 2008. These applications are hereby incorporated by reference.

“Introducing into a cell,” when referring to a dsRNA, means facilitating uptake or absorption into the cell, as is understood by those skilled in the art. Absorption or uptake of dsRNA can occur through unaided diffusive or active cellular processes, or by auxiliary agents or devices. The meaning of this term is not limited to cells in vitro; a dsRNA may also be “introduced into a cell,” wherein the cell is part of a living organism. In such instance, introduction into the cell will include the delivery to the organism. For example, for in vivo delivery, dsRNA can be injected into a tissue site or administered systemically. In vitro introduction into a cell includes methods known in the art such as electroporation and lipofection. Further approaches are described herein or known in the art.

The terms “silence,” “inhibit the expression of,” “down-regulate the expression of,” “suppress the expression of” and the like in as far as they refer to an APOC3 gene, herein refer to the at least partial suppression of the expression of an APOC3 gene, as manifested by a reduction of the amount of mRNA which may be isolated from a first cell or group of cells in which an APOC3 gene is transcribed and which has or have been treated such that the expression of an APOC3 gene is inhibited, as compared to a second cell or group of cells substantially identical to the first cell or group of cells but which has or have not been so treated (control cells). The degree of inhibition is usually expressed in terms of

(mRNA in control cells)−(mRNA in treated cells)/(mRNA in control cells)·100%

Alternatively, the degree of inhibition may be given in terms of a reduction of a parameter that is functionally linked to APOC3 gene expression, e.g., the amount of protein encoded by an APOC3 gene which is secreted by a cell, or the number of cells displaying a certain phenotype, e.g., apoptosis. In principle, APOC3 gene silencing may be determined in any cell expressing the target, either constitutively or by genomic engineering, and by any appropriate assay. However, when a reference is needed in order to determine whether a given dsRNA inhibits the expression of an APOC3 gene by a certain degree and therefore is encompassed by the instant invention, the assays provided in the Examples below shall serve as such reference.

For example, in certain instances, expression of an APOC3 gene is suppressed by at least about 5%, 10%, 15%, 20%, 25%, 30%, 35%, 40%, 45%, or 50% by administration of the double-stranded oligonucleotide featured in the invention. In some embodiments, an APOC3 gene is suppressed by at least about 60%, 70%, or 80% by administration of the double-stranded oligonucleotide featured in the invention. In some embodiments, an APOC3 gene is suppressed by at least about 85%, 90%, or 95% by administration of the double-stranded oligonucleotide featured in the invention.

As used herein in the context of APOC3 expression, the terms “treat,” “treatment,” and the like, refer to relief from or alleviation of pathological processes mediated by APOC3 expression. In the context of the present invention insofar as it relates to any of the other conditions recited herein below (other than pathological processes mediated by APOC3 expression), the terms “treat,” “treatment,” and the like mean to relieve or alleviate at least one symptom associated with such condition, or to slow or reverse the progression of such condition.

As used herein, the phrases “effective amount” refers to an amount that provides a therapeutic benefit in the treatment, prevention, or management of pathological processes mediated by APOC3 expression or an overt symptom of pathological processes mediated by APOC3 expression. The specific amount that is effective can be readily determined by an ordinary medical practitioner, and may vary depending on factors known in the art, such as, for example, the type of pathological processes mediated by APOC3 expression, the patient's history and age, the stage of pathological processes mediated by APOC3 expression, and the administration of other anti-pathological processes mediated by APOC3 expression agents.

As used herein, a “pharmaceutical composition” comprises a pharmacologically effective amount of a dsRNA and a pharmaceutically acceptable carrier. As used herein, “pharmacologically effective amount,” “therapeutically effective amount” or simply “effective amount” refers to that amount of an RNA effective to produce the intended pharmacological, therapeutic or preventive result. For example, if a given clinical treatment is considered effective when there is at least a 25% reduction in a measurable parameter associated with a disease or disorder, a therapeutically effective amount of a drug for the treatment of that disease or disorder is the amount necessary to effect at least a 25% reduction in that parameter. For example, a therapeutically effective amount of a dsRNA targeting APOC3 can reduce APOC3 serum levels by at least 25%.

The term “pharmaceutically acceptable carrier” refers to a carrier for administration of a therapeutic agent. Such carriers include, but are not limited to, saline, buffered saline, dextrose, water, glycerol, ethanol, and combinations thereof. The term specifically excludes cell culture medium. For drugs administered orally, pharmaceutically acceptable carriers include, but are not limited to pharmaceutically acceptable excipients such as inert diluents, disintegrating agents, binding agents, lubricating agents, sweetening agents, flavoring agents, coloring agents and preservatives. Suitable inert diluents include sodium and calcium carbonate, sodium and calcium phosphate, and lactose, while corn starch and alginic acid are suitable disintegrating agents. Binding agents may include starch and gelatin, while the lubricating agent, if present, will generally be magnesium stearate, stearic acid or talc. If desired, the tablets may be coated with a material such as glyceryl monostearate or glyceryl distearate, to delay absorption in the gastrointestinal tract.

As used herein, a “transformed cell” is a cell into which a vector has been introduced from which a dsRNA molecule may be expressed.

Double-Stranded Ribonucleic Acid (dsRNA)

As described in more detail herein, the invention provides double-stranded ribonucleic acid (dsRNA) molecules for inhibiting the expression of an APOC3 gene in a cell or mammal, where the dsRNA includes an antisense strand having a region of complementarity which is complementary to at least a part of an mRNA formed in the expression of an APOC3 gene, and where the region of complementarity is less than 30 nucleotides in length, generally 19-24 nucleotides in length, and where said dsRNA, upon contact with a cell expressing said APOC3 gene, inhibits the expression of said APOC3 gene by at least 30% as assayed by, for example, a PCR or branched DNA (bDNA)-based method, or by a protein-based method, such as by Western blot. Expression of an APOC3 gene can be reduced by at least 30% when measured by an assay as described in the Examples below. For example, expression of an APOC3 gene in cell culture, such as in Hep3B cells, can be assayed by measuring APOC3 mRNA levels, such as by bDNA or TaqMan assay, or by measuring protein levels, such as by ELISA assay. The dsRNA of the invention can further include one or more single-stranded nucleotide overhangs.

The dsRNA can be synthesized by standard methods known in the art as further discussed below, e.g., by use of an automated DNA synthesizer, such as are commercially available from, for example, Biosearch, Applied Biosystems, Inc. The dsRNA includes two RNA strands that are sufficiently complementary to hybridize to form a duplex structure. One strand of the dsRNA (the antisense strand) includes a region of complementarity that is substantially complementary, and generally fully complementary, to a target sequence, derived from the sequence of an mRNA formed during the expression of an APOC3 gene, the other strand (the sense strand) includes a region that is complementary to the antisense strand, such that the two strands hybridize and form a duplex structure when combined under suitable conditions. Generally, the duplex structure is between 15 and 30 or between 25 and 30, or between 18 and 25, or between 19 and 24, or between 19 and 21, or 19, 20, or 21 base pairs in length. In one embodiment the duplex is 19 base pairs in length. In another embodiment the duplex is 21 base pairs in length. When two different siRNAs are used in combination, the duplex lengths can be identical or can differ.

Each strand of the dsRNA of invention is generally between 15 and 30, or between 18 and 25, or 18, 19, 20, 21, 22, 23, 24, or 25 nucleotides in length. In other embodiments, each is strand is 25-30 nucleotides in length. Each strand of the duplex can be the same length or of different lengths. When two different siRNAs are used in combination, the lengths of each strand of each siRNA can be identical or can differ.

The dsRNA of the invention include dsRNA that are longer than 21-23 nucleotides, e.g., dsRNA that are long enough to be processed by the RNase III enzyme Dicer into 21-23 basepair siRNA which are then incorporated into a RISC. Accordingly, a dsRNA of the invention can be at least 25, 26, 27, 28, 29, 30, 40, 50, 60, 70, 80, 90, or at least 100 basepairs in length.

The dsRNA of the invention can include one or more single-stranded overhang(s) of one or more nucleotides. In one embodiment, at least one end of the dsRNA has a single-stranded nucleotide overhang of 1 to 4, generally 1 or 2 nucleotides. In another embodiment, the antisense strand of the dsRNA has 1-10 nucleotides overhangs each at the 3′ end and the 5′ end over the sense strand. In further embodiments, the sense strand of the dsRNA has 1-10 nucleotides overhangs each at the 3′ end and the 5′ end over the antisense strand.

A dsRNAs having at least one nucleotide overhang can have unexpectedly superior inhibitory properties than the blunt-ended counterpart. In some embodiments the presence of only one nucleotide overhang strengthens the interference activity of the dsRNA, without affecting its overall stability. A dsRNA having only one overhang has proven particularly stable and effective in vivo, as well as in a variety of cells, cell culture mediums, blood, and serum. Generally, the single-stranded overhang is located at the 3′-terminal end of the antisense strand or, alternatively, at the 3′-terminal end of the sense strand. The dsRNA can also have a blunt end, generally located at the 5′-end of the antisense strand. Such dsRNAs can have improved stability and inhibitory activity, thus allowing administration at low dosages, i.e., less than 5 mg/kg body weight of the recipient per day. Generally, the antisense strand of the dsRNA has a nucleotide overhang at the 3′-end, and the 5′-end is blunt. In another embodiment, one or more of the nucleotides in the overhang is replaced with a nucleoside thiophosphate.

In one embodiment, an APOC3 gene is a human APOC3 gene. In specific embodiments, the sense strand of the dsRNA is one of the sense sequences from Tables 1, 2, 6, 7, 11 or 12, and the antisense strand is one of the antisense sequences of Tables 1, 2, 6, 7, 11 or 12. Alternative antisense agents that target elsewhere in the target sequence provided in Tables 1, 2, 6, 7, 11 or 12 can readily be determined using the target sequence and the flanking APOC3 sequence.

The skilled person is well aware that dsRNAs having a duplex structure of between 20 and 23, but specifically 21, base pairs have been hailed as particularly effective in inducing RNA interference (Elbashir et al., EMBO 2001, 20:6877-6888). However, others have found that shorter or longer dsRNAs can be effective as well. In the embodiments described above, by virtue of the nature of the oligonucleotide sequences provided in Tables 1, 2, 6, 7, 11 or 12, the dsRNAs featured in the invention can include at least one strand of a length described herein. It can be reasonably expected that shorter dsRNAs having one of the sequences of Tables 1, 2, 6, 7, 11 or 12 minus only a few nucleotides on one or both ends may be similarly effective as compared to the dsRNAs described above. Hence, dsRNAs having a partial sequence of at least 15, 16, 17, 18, 19, 20, or more contiguous nucleotides from one of the sequences of Tables 1, 2, 6, 7, 11 or 12, and differing in their ability to inhibit the expression of an APOC3 gene in an assay as described herein below by not more than 5, 10, 15, 20, 25, or 30% inhibition from a dsRNA comprising the full sequence, are contemplated by the invention. Further, dsRNAs that cleave within a desired APOC3 target sequence can readily be made using the corresponding APOC3 antisense sequence and a complementary sense sequence.

In addition, the dsRNAs provided in Tables 1, 2, 6, 7, 11 or 12 identify a site in an APOC3 that is susceptible to RNAi based cleavage. As such, the present invention further features dsRNAs that target within the sequence targeted by one of the agents of the present invention. As used herein, a second dsRNA is said to target within the sequence of a first dsRNA if the second dsRNA cleaves the message anywhere within the mRNA that is complementary to the antisense strand of the first dsRNA. Such a second dsRNA will generally consist of at least 15 contiguous nucleotides from one of the sequences provided in Tables 1, 2, 6, 7, 11 or 12 coupled to additional nucleotide sequences taken from the region contiguous to the selected sequence in an APOC3 gene.

Cleavage of the RNA target can be routinely detected by gel electrophoresis and, if necessary, associated nucleic acid hybridization techniques known in the art. The cleavage site on the target mRNA of a dsRNA can be determined using methods generally known to one of ordinary skill in the art, e.g., the 5′-RACE method described in Soutschek et al., Nature; 2004, Vol. 432, pp. 173-178 (which is herein incorporated by reference for all purposes).

The dsRNA featured in the invention can contain one or more mismatches to the target sequence. In one embodiment, the dsRNA featured in the invention contains no more than 3 mismatches. If the antisense strand of the dsRNA contains mismatches to a target sequence, it is preferable that the area of mismatch not be located in the center of the region of complementarity. If the antisense strand of the dsRNA contains mismatches to the target sequence, it is preferable that the mismatch be restricted to 5 nucleotides from either end, for example 5, 4, 3, 2, or 1 nucleotide from either the 5′ or 3′ end of the region of complementarity. For example, for a 23 nucleotide dsRNA strand which is complementary to a region of an APOC3 gene, the dsRNA generally does not contain any mismatch within the central 13 nucleotides. The methods described within the invention can be used to determine whether a dsRNA containing a mismatch to a target sequence is effective in inhibiting the expression of an APOC3 gene. Consideration of the efficacy of dsRNAs with mismatches in inhibiting expression of an APOC3 gene is important, especially if the particular region of complementarity in an APOC3 gene is known to have polymorphic sequence variation within the population.

In another aspect, the invention is a single-stranded antisense oligonucleotide RNAi. An antisense oligonucleotide is a single-stranded oligonucleotide that is complementary to a sequence within the target mRNA. Antisense oligonucleotides can inhibit translation in a stoichiometric manner by base pairing to the mRNA and physically obstructing the translation machinery, see Dias, N. et al., (2002) Mol. Cancer Ther. 1:347-355. Antisense oligonucleotides can also inhibit target protein expression by binding to the mRNA target and promoting mRNA target destruction via RNase-H. The single-stranded antisense RNA molecule can be about 13 to about 30 nucleotides in length and have a sequence that is complementary to a target sequence. For example, the single-stranded antisense RNA molecule can comprise a sequence that is at least about 13, 14, 15, 16, 17, 18, 19, 20, or more contiguous nucleotides from one of the antisense sequences in Table 1, 2, 6, 7, or 10.

Modifications

In yet another embodiment, the dsRNA is chemically modified to enhance stability. The nucleic acids featured in the invention may be synthesized and/or modified by methods well established in the art, such as those described in “Current protocols in nucleic acid chemistry,” Beaucage, S. L. et al. (Eds.), John Wiley & Sons, Inc., New York, NY, USA, which is hereby incorporated herein by reference. Specific examples of dsRNA compounds useful in this invention include dsRNAs containing modified backbones or no natural internucleoside linkages. As defined in this specification, dsRNAs having modified backbones include those that retain a phosphorus atom in the backbone and those that do not have a phosphorus atom in the backbone. For the purposes of this specification, and as sometimes referenced in the art, modified dsRNAs that do not have a phosphorus atom in their internucleoside backbone can also be considered to be oligonucleosides.

Modified dsRNA backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those) having inverted polarity wherein the adjacent pairs of nucleoside units are linked 3′-5′ to 5′-3′ or 2′-5′ to 5′-2′. Various salts, mixed salts and free acid forms are also included.

Representative U.S. patents that teach the preparation of the above phosphorus-containing linkages include, but are not limited to, U.S. Pat. Nos. 3,687,808; 4,469,863; 4,476,301; 5,023,243; 5,177,195; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,316; 5,550,111; 5,563,253; 5,571,799; 5,587,361; and 5,625,050, each of which is herein incorporated by reference

Modified dsRNA backbones that do not include a phosphorus atom therein have backbones that are formed by short chain alkyl or cycloalkyl internucleoside linkages, mixed heteroatoms and alkyl or cycloalkyl internucleoside linkages, or ore or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Representative U.S. patents that teach the preparation of the above oligonucleosides include, but are not limited to, U.S. Pat. Nos. 5,034,506; 5,166,315; 5,185,444; 5,214,134; 5,216,141; 5,235,033; 5,64,562; 5,264,564; 5,405,938; 5,434,257; 5,466,677; 5,470,967; 5,489,677; 5,541,307; 5,561,225; 5,596,086; 5,602,240; 5,608,046; 5,610,289; 5,618,704; 5,623,070; 5,663,312; 5,633,360; 5,677,437; and, 5,677,439, each of which is herein incorporated by reference.

In other suitable dsRNA mimetics, both the sugar and the internucleoside linkage, i.e., the backbone, of the nucleotide units are replaced with novel groups. The base units are maintained for hybridization with an appropriate nucleic acid target compound. One such oligomeric compound, a dsRNA mimetic that has been shown to have excellent hybridization properties, is referred to as a peptide nucleic acid (PNA). In PNA compounds, the sugar backbone of a dsRNA is replaced with an amide containing backbone, in particular an aminoethylglycine backbone. The nucleobases are retained and are bound directly or indirectly to aza nitrogen atoms of the amide portion of the backbone. Representative U.S. patents that teach the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al., Science, 1991, 254, 1497-1500.

Other embodiments of the invention are dsRNAs with phosphorothioate backbones and oligonucleosides with heteroatom backbones, and in particular —CH₂—NH—CH₂—, —CH₂—N(CH₃)—O—CH₂—[known as a methylene (methylimino) or MMI backbone], —CH₂—O—N(CH₃)—CH₂—, —CH₂—N(CH₃)—N(CH₃)—CH₂— and —N(CH₃)—CH₂—CH₂—[wherein the native phosphodiester backbone is represented as —O—P—O—CH₂—] of the above-referenced U.S. Pat. No. 5,489,677, and the amide backbones of the above-referenced U.S. Pat. No. 5,602,240. Also preferred are dsRNAs having morpholino backbone structures of the above-referenced U.S. Pat. No. 5,034,506.

Modified dsRNAs may also contain one or more substituted sugar moieties. Preferred dsRNAs comprise one of the following at the 2′ position: OH; F; O—, S—, or N-alkyl; O—, S—, or N-alkenyl; O- , S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Particularly preferred are O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. Other preferred dsRNAs comprise one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of a dsRNA, or a group for improving the pharmacodynamic properties of a dsRNA, and other substituents having similar properties. A preferred modification includes 2′-methoxyethoxy (2′-O—CH₂CH₂OCH₃, also known as 2′-O-(2-methoxyethyl) or 2′-MOE) (Martin et al., Helv. Chim. Acta, 1995, 78, 486-504) i.e., an alkoxy-alkoxy group. A further preferred modification includes 2′-dimethylaminooxyethoxy, i.e., a O(CH₂)₂ON(CH₃)₂ group, also known as 2′-DMAOE, as described in examples herein below, and 2′-dimethylaminoethoxyethoxy (also known in the art as 2′-O-dimethylaminoethoxyethyl or 2′-DMAEOE), i.e., 2′-O—CH₂—O—CH₂—N(CH₂)₂, also described in examples herein below.

Other preferred modifications include 2′-methoxy (2′-OCH₃), 2′-aminopropoxy (2′-OCH₂CH₂CH₂NH₂) and 2′-fluoro (2′-F). Similar modifications may also be made at other positions on the dsRNA, particularly the 3′ position of the sugar on the 3′ terminal nucleotide or in 2′-5′ linked dsRNAs and the 5′ position of 5′ terminal nucleotide. DsRNAs may also have sugar mimetics such as cyclobutyl moieties in place of the pentofuranosyl sugar. Representative U.S. patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, certain of which are commonly owned with the instant application, and each of which is herein incorporated by reference in its entirety.

dsRNAs may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl anal other 8-substituted adenines and guanines, 5-halo, particularly 5-bromo, 5-trifluoromethyl and other 5-substituted uracils and cytosines, 7-methylguanine and 7-methyladenine, 8-azaguanine and 8-azaadenine, 7-deazaguanine and 7-daazaadenine and 3-deazaguanine and 3-deazaadenine. Further nucleobases include those disclosed in U.S. Pat. No. 3,687,808, those disclosed in The Concise Encyclopedia Of Polymer Science And Engineering, pages 858-859, Kroschwitz, J. L, ed. John Wiley & Sons, 1990, these disclosed by Englisch et al., Angewandte Chemie, International Edition, 1991, 30, 613, and those disclosed by Sanghvi, Y S., Chapter 15, DsRNA Research and Applications, pages 289-302, Crooke, S. T. and Lebleu, B., Ed., CRC Press, 1993. Certain of these nucleobases are particularly useful for increasing the binding affinity of the oligomeric compounds featured in the invention. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., Eds., DsRNA Research and Applications, CRC Press, Boca Raton, 1993, pp. 276-278) and are exemplary base substitutions, even more particularly when combined with 2′-O-methoxyethyl sugar modifications.

Representative U.S. patents that teach the preparation of certain of the above noted modified nucleobases as well as other modified nucleobases include, but are not limited to, the above noted U.S. Pat. No. 3,687,808, as well as U.S. Pat. Nos. 4,845,205; 5,130,30; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; and 5,681,941, each of which is herein incorporated by reference, and U.S. Pat. No. 5,750,692, also herein incorporated by reference.

Conjugates

Another modification of the dsRNAs of the invention involves chemically linking to the dsRNA one or more moieties or conjugates which enhance the activity, cellular distribution or cellular uptake of the dsRNA. Such moieties include but are not limited to lipid moieties such as a cholesterol moiety (Letsinger et al., Proc. Natl. Acid. Sci. USA, 1989, 86: 6553-6556), cholic acid (Manoharan et al., Biorg. Med. Chem. Let., 1994, 4:1053-1060), a thioether, e.g., beryl-S-tritylthiol (Manoharan et al., Ann. N.Y. Acad. Sci., 1992, 660:306-309; Manoharan et al., Biorg. Med. Chem. Let., 1993, 3:2765-2770), a thiocholesterol (Oberhauser et al., Nucl. Acids Res., 1992, 20:533-538), an aliphatic chain, e.g., dodecandiol or undecyl residues (Saison-Behmoaras et al., EMBO J, 1991, 10:1111-1118; Kabanov et al., FEBS Lett., 1990, 259:327-330; Svinarchuk et al., Biochimie, 1993, 75:49-54), a phospholipid, e.g., di-hexadecyl-rac-glycerol or triethyl-ammonium 1,2-di-O-hexadecyl-rac-glycero-3-Hphosphonate (Manoharan et al., Tetrahedron Lett., 1995, 36:3651-3654; Shea et al., Nucl. Acids Res., 1990, 18:3777-3783), a polyamine or a polyethylene glycol chain (Manoharan et al., Nucleosides & Nucleotides, 1995, 14:969-973), or adamantane acetic acid (Manoharan et al., Tetrahedron Lett., 1995, 36:3651-3654), a palmityl moiety (Mishra et al., Biochim. Biophys. Acta, 1995, 1264:229-237), or an octadecylamine or hexylamino-carbonyloxycholesterol moiety (Crooke et al., J. Pharmacol. Exp. Ther., 1996, 277:923-937).

It is not necessary for all positions in a given compound to be uniformly modified, and in fact more than one of the aforementioned modifications may be incorporated in a single compound or even at a single nucleoside within a dsRNA. The present invention also includes dsRNA compounds which are chimeric compounds. “Chimeric” dsRNA compounds or “chimeras,” in the context of this invention, are dsRNA compounds, particularly dsRNAs, which contain two or more chemically distinct regions, each made up of at least one monomer unit, i.e., a nucleotide in the case of a dsRNA compound. These dsRNAs typically contain at least one region wherein the dsRNA is modified so as to confer upon the dsRNA increased resistance to nuclease degradation, increased cellular uptake, and/or increased binding affinity for the target nucleic acid. An additional region of the dsRNA may serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of dsRNA inhibition of gene expression. Consequently, comparable results can often be obtained with shorter dsRNAs when chimeric dsRNAs are used, compared to phosphorothioate deoxy dsRNAs hybridizing to the same target region.

In certain instances, the dsRNA may be modified by a non-ligand group. A number of non-ligand molecules have been conjugated to dsRNAs in order to enhance the activity, cellular distribution or cellular uptake of the dsRNA, and procedures for performing such conjugations are available in the scientific literature. Such non-ligand moieties have included lipid moieties, such as cholesterol (Letsinger et al., Proc. Natl. Acad. Sci. USA, 1989, 86:6553), cholic acid (Manoharan et al., Bioorg. Med. Chem. Lett., 1994, 4:1053), a thioether, e.g., hexyl-S-tritylthiol (Manoharan et al., Ann. N.Y. Acad. Sci., 1992, 660:306; Manoharan et al., Bioorg. Med. Chem. Let., 1993, 3:2765), a thiocholesterol (Oberhauser et al., Nucl. Acids Res., 1992, 20:533), an aliphatic chain, e.g., dodecandiol or undecyl residues (Saison-Behmoaras et al., EMBO J., 1991, 10:111; Kabanov et al., FEBS Lett., 1990, 259:327; Svinarchuk et al., Biochimie, 1993, 75:49), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36:3651; Shea et al., Nucl. Acids Res., 1990, 18:3777), a polyamine or a polyethylene glycol chain (Manoharan et al., Nucleosides & Nucleotides, 1995, 14:969), or adamantane acetic acid (Manoharan et al., Tetrahedron Lett., 1995, 36:3651), a palmityl moiety (Mishra et al., Biochim. Biophys. Acta, 1995, 1264:229), or an octadecylamine or hexylamino-carbonyl-oxycholesterol moiety (Crooke et al., J. Pharmacol. Exp. Ther., 1996, 277:923). Representative United States patents that teach the preparation of such dsRNA conjugates have been listed above. Typical conjugation protocols involve the synthesis of dsRNAs bearing an aminolinker at one or more positions of the sequence. The amino group is then reacted with the molecule being conjugated using appropriate coupling or activating reagents. The conjugation reaction may be performed either with the dsRNA still bound to the solid support or following cleavage of the dsRNA in solution phase. Purification of the dsRNA conjugate by HPLC typically affords the pure conjugate.

Conjugating a ligand to a dsRNA can enhance its cellular absorption as well as targeting to a particular tissue or uptake by specific types of cells such as liver cells. In certain instances, a hydrophobic ligand is conjugated to the dsRNA to facilitate direct permeation of the cellular membrane and or uptake across the liver cells. Alternatively, the ligand conjugated to the dsRNA is a substrate for receptor-mediated endocytosis. These approaches have been used to facilitate cell permeation of antisense oligonucleotides as well as dsRNA agents. For example, cholesterol has been conjugated to various antisense oligonucleotides resulting in compounds that are substantially more active compared to their non-conjugated analogs. See M. Manoharan Antisense & Nucleic Acid Drug Development 2002, 12, 103. Other lipophilic compounds that have been conjugated to oligonucleotides include 1-pyrene butyric acid, 1,3-bis-O-(hexadecyl)glycerol, and menthol. One example of a ligand for receptor-mediated endocytosis is folic acid. Folic acid enters the cell by folate-receptor-mediated endocytosis. dsRNA compounds bearing folic acid would be efficiently transported into the cell via the folate-receptor-mediated endocytosis. Li and coworkers report that attachment of folic acid to the 3′-terminus of an oligonucleotide resulted in an 8-fold increase in cellular uptake of the oligonucleotide. Li, S.; Deshmukh, H. M.; Huang, L. Pharm. Res. 1998, 15, 1540. Other ligands that have been conjugated to oligonucleotides include polyethylene glycols, carbohydrate clusters, cross-linking agents, porphyrin conjugates, delivery peptides and lipids such as cholesterol and cholesterylamine. Examples of carbohydrate clusters include Chol-p-(GalNAc)₃ (N-acetyl galactosamine cholesterol) and LCO(GalNAc)₃ (N-acetyl galactosamine-3′-Lithocholic-oleoyl.

Carbohydrate Conjugates

In Some Embodiments of the Compositions and Methods of the Invention, a dsRNA oligonucleotide further comprises a carbohydrate. The carbohydrate conjugated dsRNA are advantageous for the in vivo delivery of nucleic acids, as well as compositions suitable for in vivo therapeutic use, as described herein. As used herein, “carbohydrate” refers to a compound which is either a carbohydrate per se made up of one or more monosaccharide units having at least 6 carbon atoms (which can be linear, branched or cyclic) with an oxygen, nitrogen or sulfur atom bonded to each carbon atom; or a compound having as a part thereof a carbohydrate moiety made up of one or more monosaccharide units each having at least six carbon atoms (which can be linear, branched or cyclic), with an oxygen, nitrogen or sulfur atom bonded to each carbon atom. Representative carbohydrates include the sugars (mono-, di-, tri- and oligosaccharides containing from about 4, 5, 6, 7, 8, or 9 monosaccharide units), and polysaccharides such as starches, glycogen, cellulose and polysaccharide gums. Specific monosaccharides include C5 and above (e.g., C5, C6, C7, or C8) sugars; di- and trisaccharides include sugars having two or three monosaccharide units (e.g., C5, C6, C7, or C8).

In one embodiment, a carbohydrate conjugate for use in the compositions and methods of the invention is a monosaccharide. In one embodiment, the monosaccharide is an N-acetylgalactosamine, such as

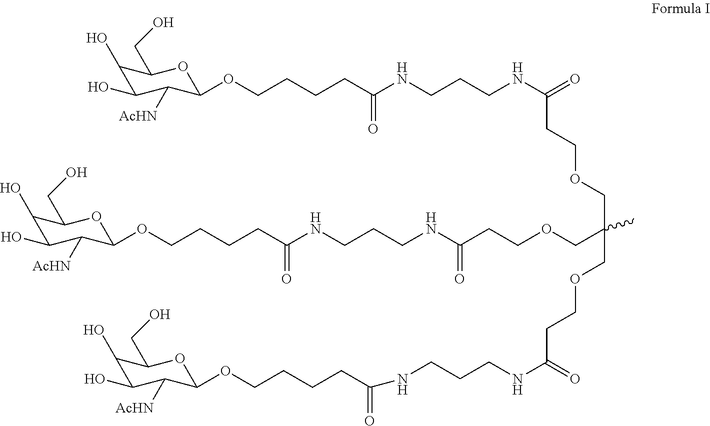 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

In another embodiment, a carbohydrate conjugate for use in the compositions and methods of the invention is selected from the group consisting of:

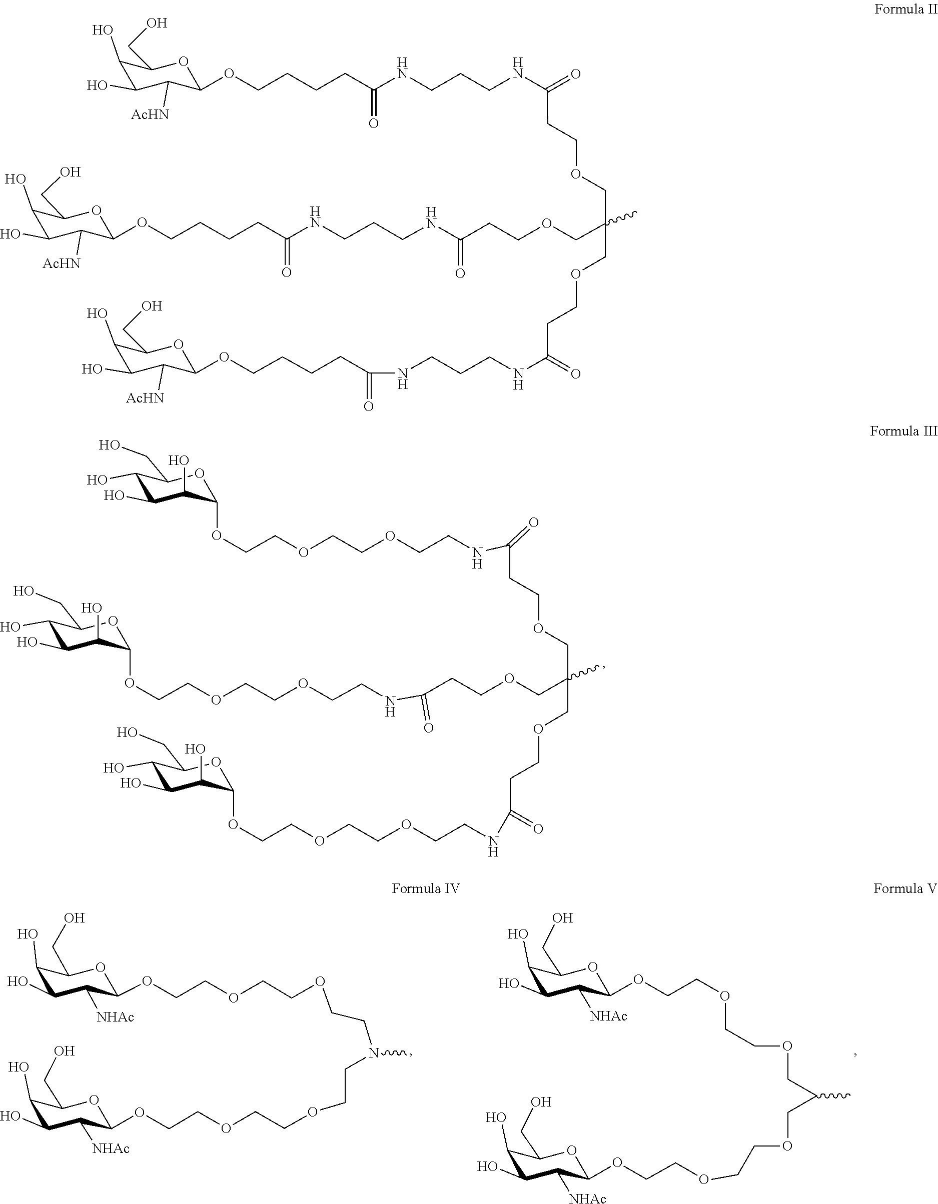

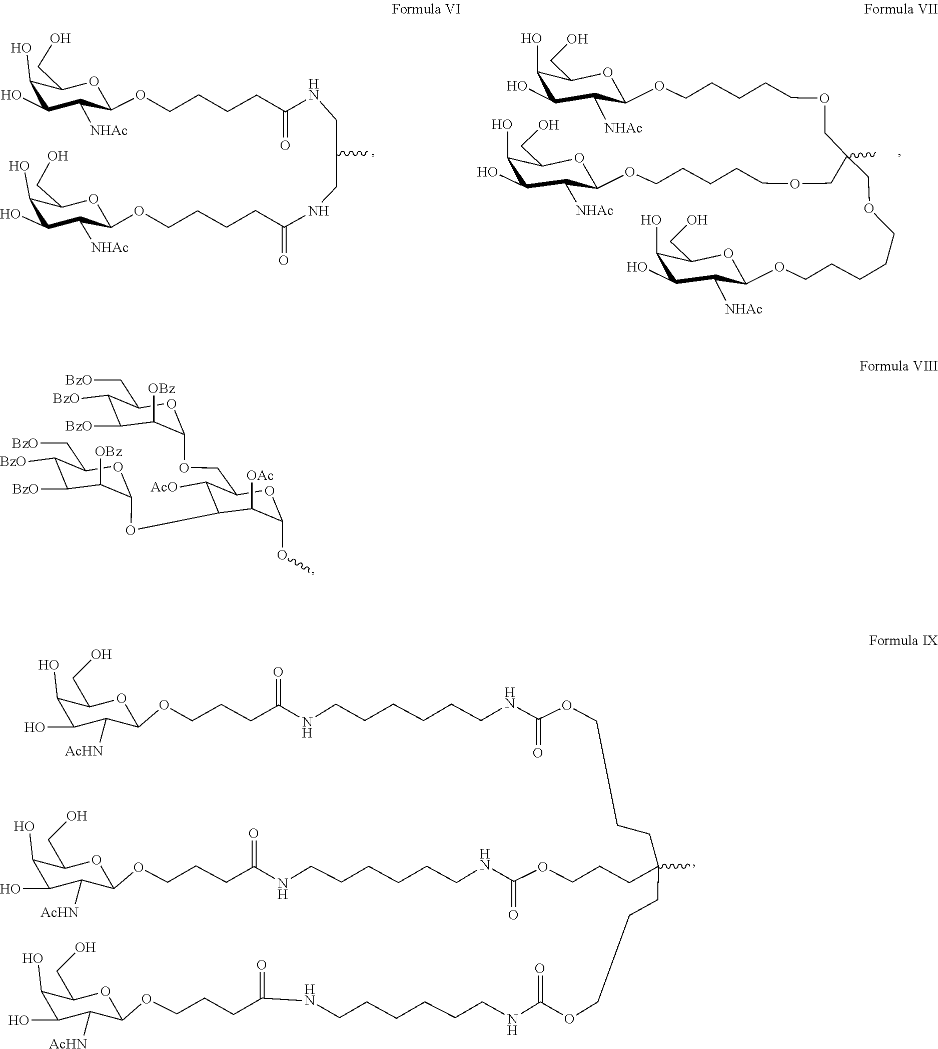

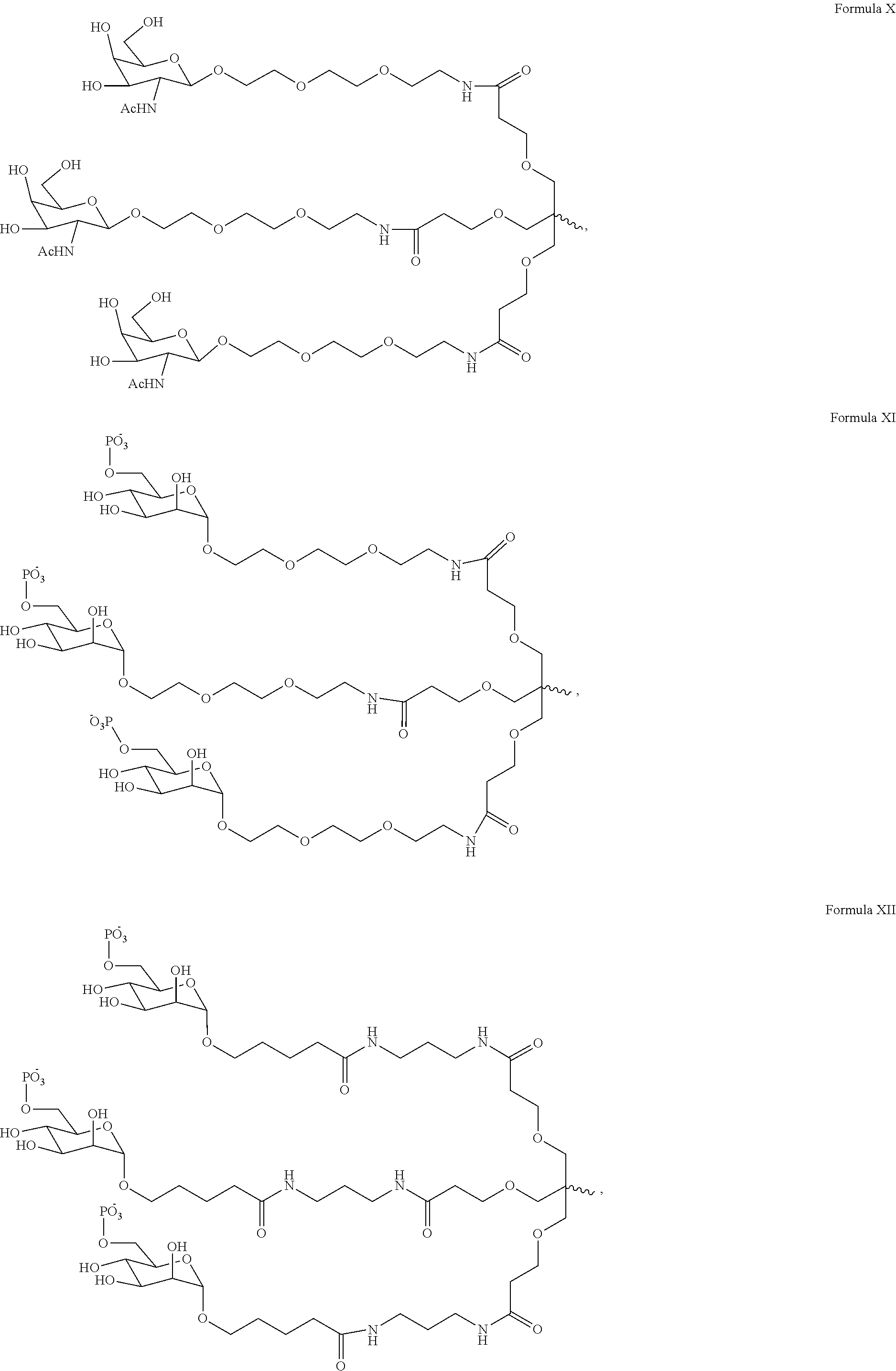

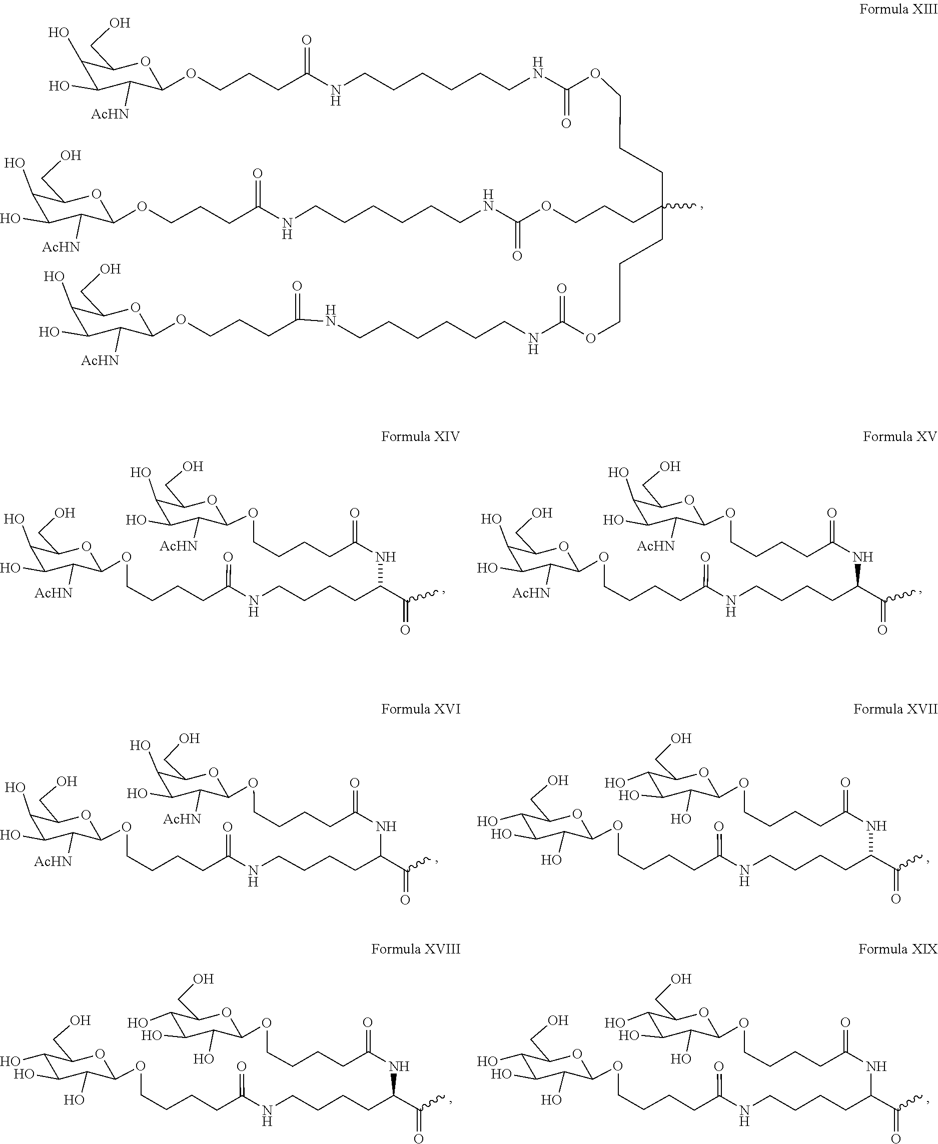

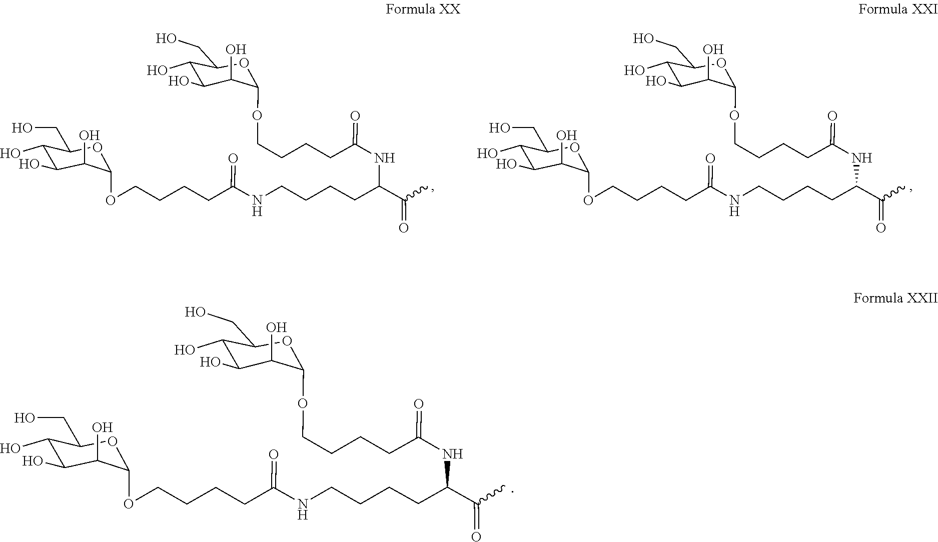

Another representative carbohydrate conjugate for use in the embodiments described herein includes, but is not limited to,

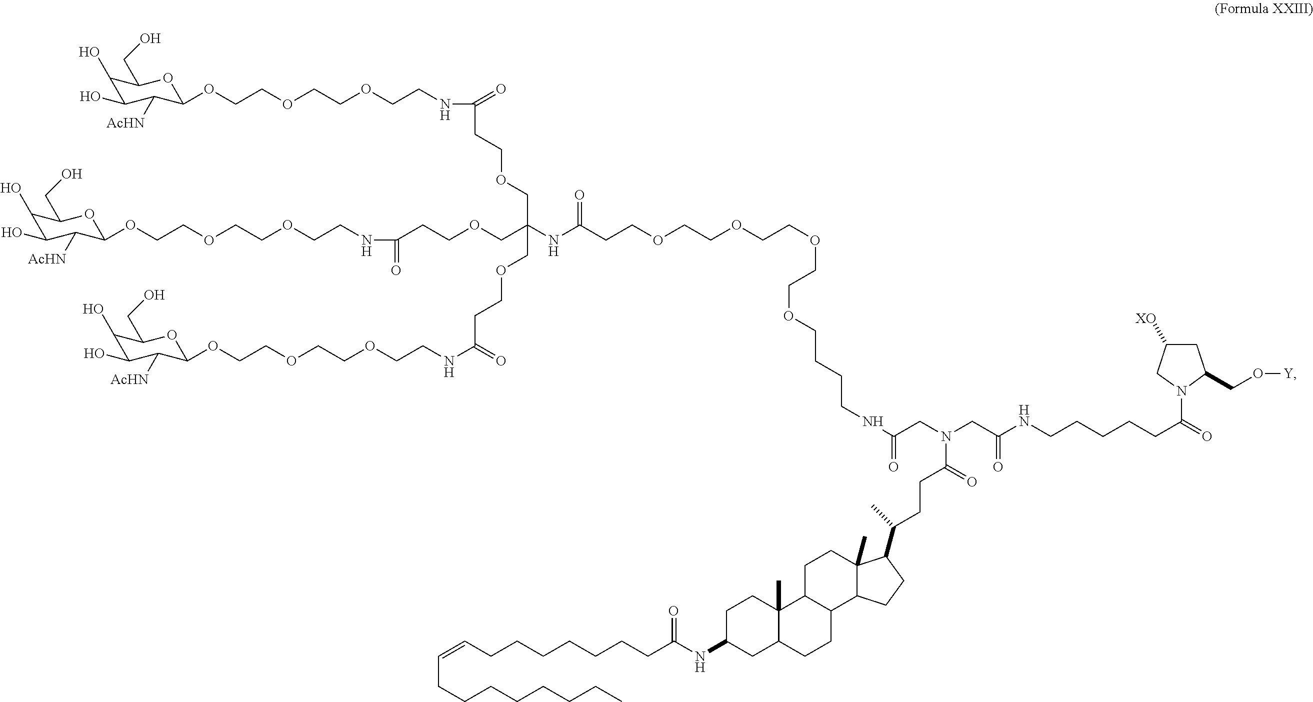

when one of X or Y is an oligonucleotide, the other is a hydrogen.

In some embodiments, the carbohydrate conjugate further comprises one or more additional ligands as described above, such as, but not limited to, a PK modulator and/or a cell permeation peptide.

Linkers

In some embodiments, the conjugate or ligand described herein can be attached to a dsRNA of the invention with various linkers that can be cleavable or non cleavable.

The term “linker” or “linking group” means an organic moiety that connects two parts of a compound, e.g., covalently attaches two parts of a compound. Linkers typically comprise a direct bond or an atom such as oxygen or sulfur, a unit such as NR8, C(O), C(O)NH, SO, SO₂, SO₂NH or a chain of atoms, such as, but not limited to, substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, alkynylhereroaryl, which one or more methylenes can be interrupted or terminated by O, S, S(O), SO₂, N(R8), C(O), substituted or unsubstituted aryl, substituted or unsubstituted heteroaryl, substituted or unsubstituted heterocyclic; where R8 is hydrogen, acyl, aliphatic or substituted aliphatic. In one embodiment, the linker is between about 1-24 atoms, 2-24, 3-24, 4-24, 5-24, 6-24, 6-18, 7-18, 8-18 atoms, 7-17, 8-17, 6-16, 7-17, or 8-16 atoms.

A cleavable linking group is one which is sufficiently stable outside the cell, but which upon entry into a target cell is cleaved to release the two parts the linker is holding together. In a preferred embodiment, the cleavable linking group is cleaved at least about 10 times, 20, times, 30 times, 40 times, 50 times, 60 times, 70 times, 80 times, 90 times or more, or at least about 100 times faster in a target cell or under a first reference condition (which can, e.g., be selected to mimic or represent intracellular conditions) than in the blood of a subject, or under a second reference condition (which can, e.g., be selected to mimic or represent conditions found in the blood or serum).

Cleavable linking groups are susceptible to cleavage agents, e.g., pH, redox potential or the presence of degradative molecules. Generally, cleavage agents are more prevalent or found at higher levels or activities inside cells than in serum or blood. Examples of such degradative agents include: redox agents which are selected for particular substrates or which have no substrate specificity, including, e.g., oxidative or reductive enzymes or reductive agents such as mercaptans, present in cells, that can degrade a redox cleavable linking group by reduction; esterases; endosomes or agents that can create an acidic environment, e.g., those that result in a pH of five or lower; enzymes that can hydrolyze or degrade an acid cleavable linking group by acting as a general acid, peptidases (which can be substrate specific), and phosphatases.

A cleavable linkage group, such as a disulfide bond can be susceptible to pH. The pH of human serum is 7.4, while the average intracellular pH is slightly lower, ranging from about 7.1-7.3. Endosomes have a more acidic pH, in the range of 5.5-6.0, and lysosomes have an even more acidic pH at around 5.0. Some linkers will have a cleavable linking group that is cleaved at a preferred pH, thereby releasing a cationic lipid from the ligand inside the cell, or into the desired compartment of the cell.

A linker can include a cleavable linking group that is cleavable by a particular enzyme. The type of cleavable linking group incorporated into a linker can depend on the cell to be targeted. For example, a liver-targeting ligand can be linked to a cationic lipid through a linker that includes an ester group. Liver cells are rich in esterases, and therefore the linker will be cleaved more efficiently in liver cells than in cell types that are not esterase-rich. Other cell-types rich in esterases include cells of the lung, renal cortex, and testis.

Linkers that contain peptide bonds can be used when targeting cell types rich in peptidases, such as liver cells and synoviocytes.

In general, the suitability of a candidate cleavable linking group can be evaluated by testing the ability of a degradative agent (or condition) to cleave the candidate linking group. It will also be desirable to also test the candidate cleavable linking group for the ability to resist cleavage in the blood or when in contact with other non-target tissue. Thus, one can determine the relative susceptibility to cleavage between a first and a second condition, where the first is selected to be indicative of cleavage in a target cell and the second is selected to be indicative of cleavage in other tissues or biological fluids, e.g., blood or serum. The evaluations can be carried out in cell free systems, in cells, in cell culture, in organ or tissue culture, or in whole animals. It can be useful to make initial evaluations in cell-free or culture conditions and to confirm by further evaluations in whole animals. In preferred embodiments, useful candidate compounds are cleaved at least about 2, 4, 10, 20, 30, 40, 50, 60, 70, 80, 90, or about 100 times faster in the cell (or under in vitro conditions selected to mimic intracellular conditions) as compared to blood or serum (or under in vitro conditions selected to mimic extracellular conditions).

In one embodiment, a cleavable linking group is a redox cleavable linking group that is cleaved upon reduction or oxidation. An example of reductively cleavable linking group is a disulphide linking group (—S—S—). To determine if a candidate cleavable linking group is a suitable “reductively cleavable linking group,” or for example is suitable for use with a particular dsRNA moiety and particular targeting agent one can look to methods described herein. For example, a candidate can be evaluated by incubation with dithiothreitol (DTT), or other reducing agent using reagents know in the art, which mimic the rate of cleavage which would be observed in a cell, e.g., a target cell. The candidates can also be evaluated under conditions which are selected to mimic blood or serum conditions. In one, candidate compounds are cleaved by at most about 10% in the blood. In other embodiments, useful candidate compounds are degraded at least about 2, 4, 10, 20, 30, 40, 50, 60, 70, 80, 90, or about 100 times faster in the cell (or under in vitro conditions selected to mimic intracellular conditions) as compared to blood (or under in vitro conditions selected to mimic extracellular conditions). The rate of cleavage of candidate compounds can be determined using standard enzyme kinetics assays under conditions chosen to mimic intracellular media and compared to conditions chosen to mimic extracellular media.

In another embodiment, a cleavable linker comprises a phosphate-based cleavable linking group. A phosphate-based cleavable linking group is cleaved by agents that degrade or hydrolyze the phosphate group. An example of an agent that cleaves phosphate groups in cells are enzymes such as phosphatases in cells. Examples of phosphate-based linking groups are —O—P(O)(ORk)-O—, —O—P(S)(ORk)-O—, —O—P(S)(SRk)-O—, —S—P(O)(ORk)-O—, —O—P(O)(ORk)-S—, —S—P(O)(ORk)-S—, —O—P(S)(ORk)-S—, —S—P(S)(ORk)-O—, —O—P(O)(Rk)-O—, —O—P(S)(Rk)-O—, —S—P(O)(Rk)-O—, —S—P(S)(Rk)-O—, —S—P(O)(Rk)-S—, —O—P(S)(Rk)-S—. Preferred embodiments are —O—P(O)(OH)—O—, —O—P(S)(OH)—O—, —O—P(S)(SH)—O—, —S—P(O)(OH)—O—, —O—P(O)(OH)—S—, —S—P(O)(OH)—S—, —O—P(S)(OH)—S—, —S—P(S)(OH)—O—, —O—P(O)(H)—O—, —O—P(S)(H)—O—, —S—P(O)(H)—O—, —S—P(S)(H)—O—, —S—P(O)(H)—S—, —O—P(S)(H)—S—. A preferred embodiment is —O—P(O)(OH)—O—. These candidates can be evaluated using methods analogous to those described above.

In another embodiment, a cleavable linker comprises an acid cleavable linking group. An acid cleavable linking group is a linking group that is cleaved under acidic conditions. In preferred embodiments acid cleavable linking groups are cleaved in an acidic environment with a pH of about 6.5 or lower (e.g., about 6.0, 5.75, 5.5, 5.25, 5.0, or lower), or by agents such as enzymes that can act as a general acid. In a cell, specific low pH organelles, such as endosomes and lysosomes can provide a cleaving environment for acid cleavable linking groups. Examples of acid cleavable linking groups include but are not limited to hydrazones, esters, and esters of amino acids. Acid cleavable groups can have the general formula —C═NN—, C(O)O, or —OC(O). A preferred embodiment is when the carbon attached to the oxygen of the ester (the alkoxy group) is an aryl group, substituted alkyl group, or tertiary alkyl group such as dimethyl pentyl or t-butyl. These candidates can be evaluated using methods analogous to those described above.

In another embodiment, a cleavable linker comprises an ester-based cleavable linking group. An ester-based cleavable linking group is cleaved by enzymes such as esterases and amidases in cells. Examples of ester-based cleavable linking groups include but are not limited to esters of alkylene, alkenylene and alkynylene groups. Ester cleavable linking groups have the general formula —C(O)O—, or —OC(O)—. These candidates can be evaluated using methods analogous to those described above.

In yet another embodiment, a cleavable linker comprises a peptide-based cleavable linking group. A peptide-based cleavable linking group is cleaved by enzymes such as peptidases and proteases in cells. Peptide-based cleavable linking groups are peptide bonds formed between amino acids to yield oligopeptides (e.g., dipeptides, tripeptides, etc.) and polypeptides. Peptide-based cleavable groups do not include the amide group (—C(O)NH—). The amide group can be formed between any alkylene, alkenylene or alkynelene. A peptide bond is a special type of amide bond formed between amino acids to yield peptides and proteins. The peptide based cleavage group is generally limited to the peptide bond (i.e., the amide bond) formed between amino acids yielding peptides and proteins and does not include the entire amide functional group. Peptide-based cleavable linking groups have the general formula —NHCHRAC(O)NHCHRBC(O)—(SEQ ID NO: 13), where RA and RB are the R groups of the two adjacent amino acids. These candidates can be evaluated using methods analogous to those described above.

In one embodiment, a dsRNA of the invention is conjugated to a carbohydrate through a linker. Non-limiting examples of dsRNA carbohydrate conjugates with linkers of the compositions and methods of the invention include, but are not limited to,

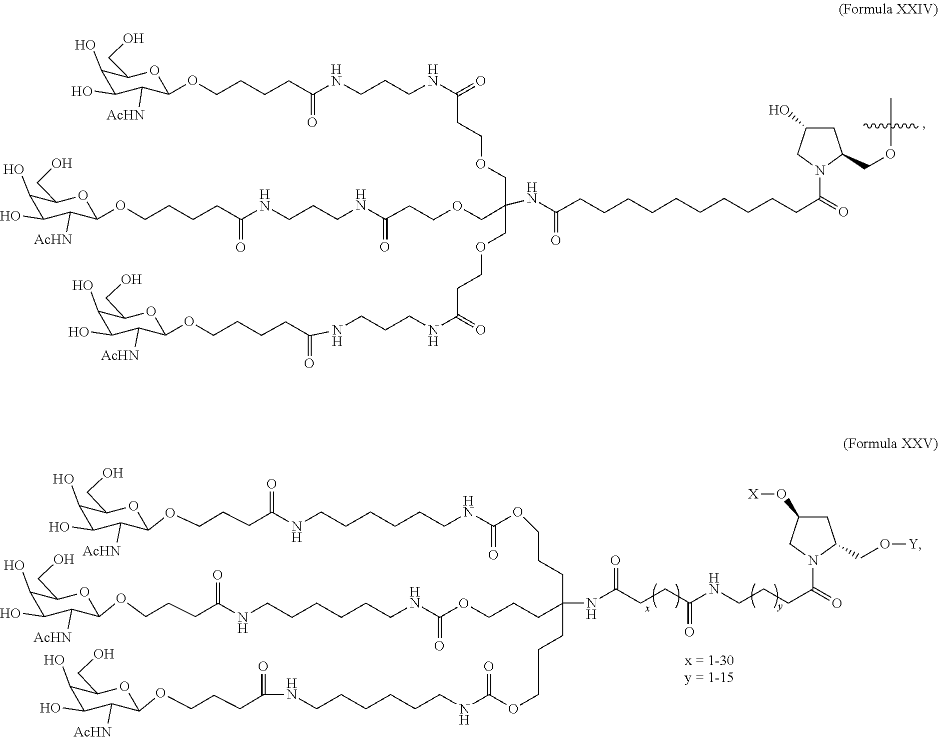

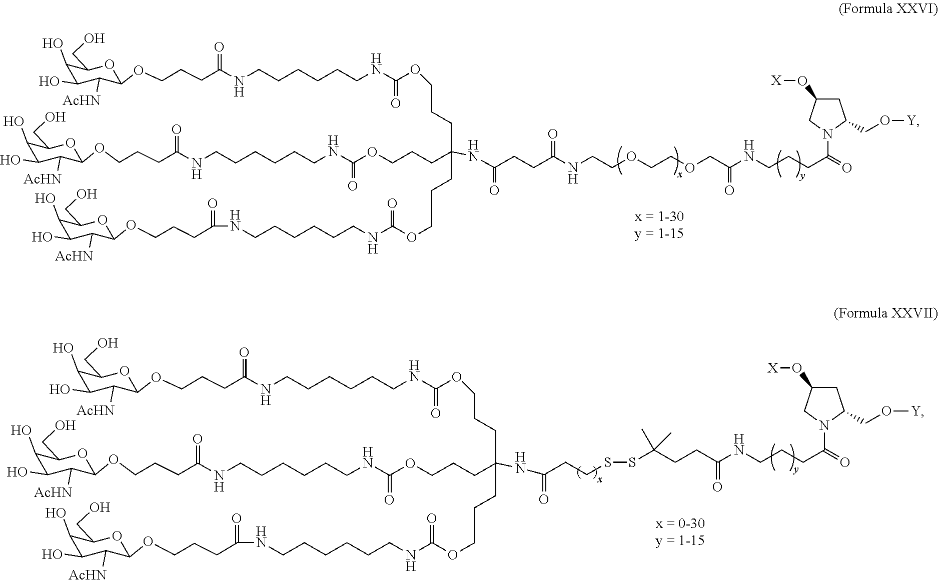

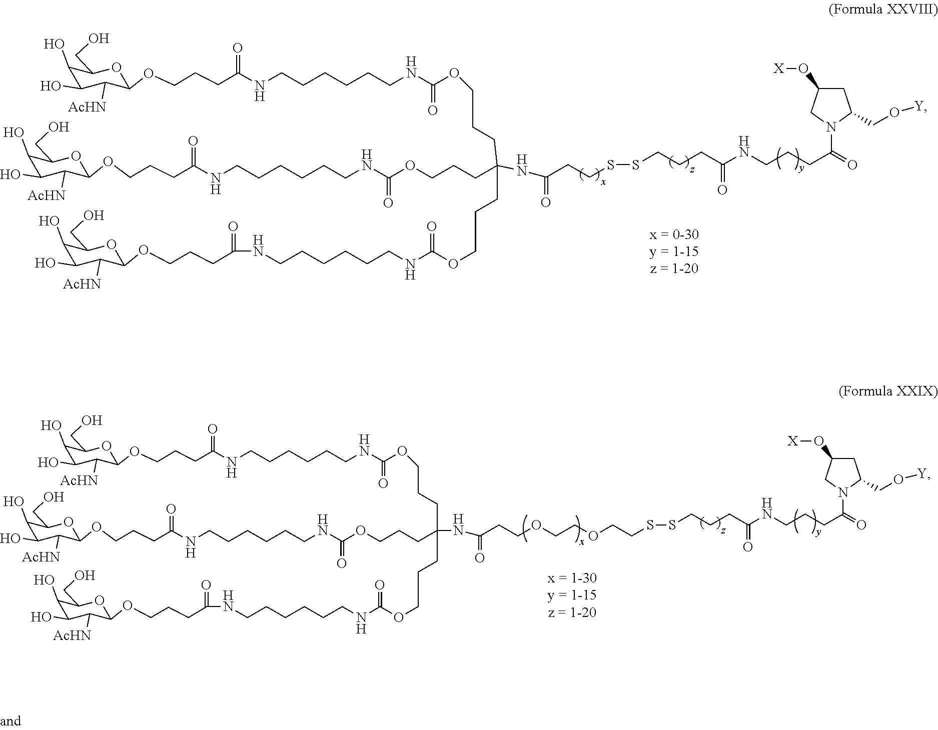

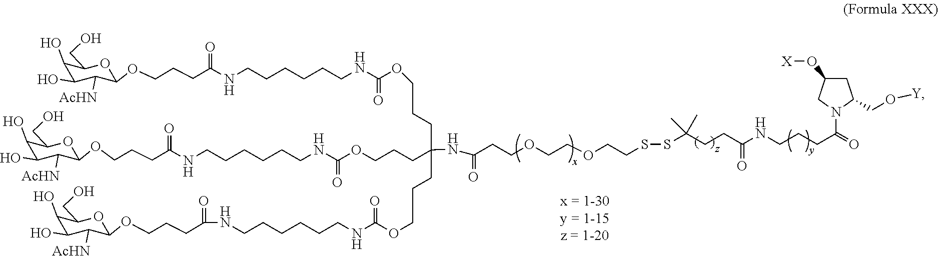

when one of X or Y is an oligonucleotide, the other is a hydrogen.

In certain embodiments of the compositions and methods of the invention, a ligand is one or more GalNAc (N-acetylgalactosamine) derivatives attached through a bivalent or trivalent branched linker.

In one embodiment, a dsRNA of the invention is conjugated to a bivalent or trivalent branched linker selected from the group of structures shown in any of formula (XXXI)-(XXXIV):

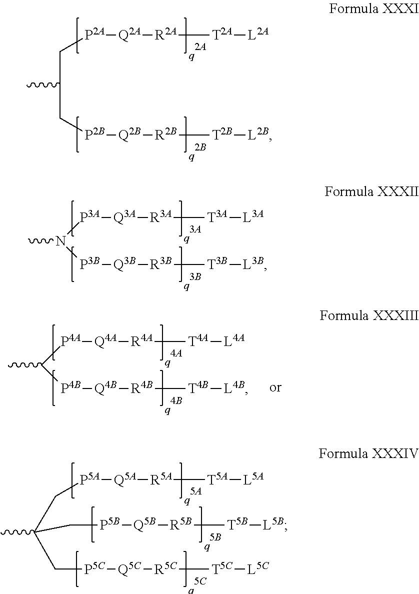

wherein: q2A, q2B, q3A, q3B, q4A, q4B, q5A, q5B and q5C represent independently for each occurrence 0-20 and wherein the repeating unit can be the same or different;
P^{2A}, P^{2B}, P^{3A}, P^{3B}, P^{4A}, P^{4B}, P^{5A}, P^{5B}, P^{5C}, T^{2A}, T^{2B}, T^{3A}, T^{3B}, T^{4A}, T^{4B}, T^{4A}, T^{5B}, T^{5C} are each independently for each occurrence absent, CO, NH, O, S, OC(O), NHC(O), CH₂, CH₂NH or CH₂O;
Q^{2A}, Q^{2B}, Q^{3A}, Q^{3B}, Q^{4A}, Q^{4B}, Q^{5A}, Q^{5B}, Q⁵C are independently for each occurrence absent, alkylene, substituted alkylene wherein one or more methylenes can be interrupted or terminated by one or more of 0, S, S(O), S02, N(R^{N}), C(R′)═C(R″), C≡C or C(O);
R^{2A}, R^{2B}, R^{3A}, R^{3B}, R^{4A}, R^{4B}, R^{5A}, R^{5B}, R^{5C} are each independently for each occurrence absent, NH, O, S, CH₂, C(O)O, C(O)NH, NHCH(R^{a})C(O), —C(O)—CH(R^{a})—NH—, CO,

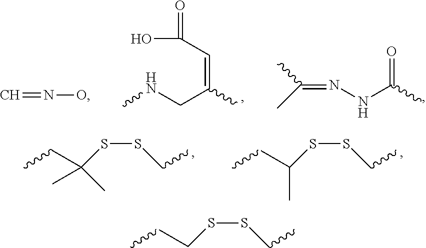

or heterocyclyl;
L^{2A}, L^{2B}, L^{3A}, L^{3B}, L^{4A}, L^{4B}, L^{5A}, L^{5B}, and L^{5C} represent the ligand; i.e. each independently for each occurrence a monosaccharide (such as GalNAc), disaccharide, trisaccharide, tetrasaccharide, oligosaccharide, or polysaccharide; and R^{a} is H or amino acid side chain. Trivalent conjugating GalNAc derivatives are particularly useful for use with RNAi agents for inhibiting the expression of a target gene, such as those of formula (XXXV):

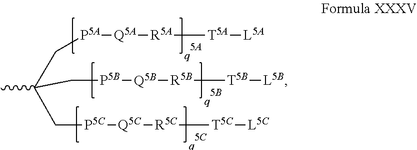

wherein L^{5A}, L^{5B} and L^{5C} represent a monosaccharide, such as GalNAc derivative.

Examples of suitable bivalent and trivalent branched linker groups conjugating GalNAc derivatives include, but are not limited to, the structures recited above as formulas II_VII, XI, X, and XIII.

Representative U.S. patents that teach the preparation of RNA conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941; 6,294,664; 6,320,017; 6,576,752; 6,783,931; 6,900,297; 7,037,646; 8,106,022, the entire contents of each of which are hereby incorporated herein by reference.

Vector Encoded dsRNAs

In another aspect, APOC3 dsRNA molecules are expressed from transcription units inserted into DNA or RNA vectors (see, e.g., Couture, A, et al., TIG. (1996), 12:5-10; Skillern, A., et al., International PCT Publication No. WO 00/22113, Conrad, International PCT Publication No. WO 00/22114, and Conrad, U.S. Pat. No. 6,054,299). These transgenes can be introduced as a linear construct, a circular plasmid, or a viral vector, which can be incorporated and inherited as a transgene integrated into the host genome. The transgene can also be constructed to permit it to be inherited as an extrachromosomal plasmid (Gassmann et al., Proc. Natl. Acad. Sci. USA (1995) 92:1292).

The individual strands of a dsRNA can be transcribed by promoters on two separate expression vectors and co-transfected into a target cell. Alternatively each individual strand of the dsRNA can be transcribed by promoters both of which are located on the same expression plasmid. In one embodiment, a dsRNA is expressed as an inverted repeat joined by a linker polynucleotide sequence such that the dsRNA has a stem and loop structure.

The recombinant dsRNA expression vectors are generally DNA plasmids or viral vectors. dsRNA expressing viral vectors can be constructed based on, but not limited to, adeno-associated virus (for a review, see Muzyczka, et al., Curr. Topics Micro. Immunol. (1992) 158:97-129)); adenovirus (see, for example, Berkner, et al., BioTechniques (1998) 6:616), Rosenfeld et al. (1991, Science 252:431-434), and Rosenfeld et al. (1992), Cell 68:143-155)); or alphavirus as well as others known in the art. Retroviruses have been used to introduce a variety of genes into many different cell types, including epithelial cells, in vitro and/or in vivo (see, e.g., Eglitis, et al., Science (1985) 230:1395-1398; Danos and Mulligan, Proc. Natl. Acad. Sci. USA (1998) 85:6460-6464; Wilson et al., 1988, Proc. Natl. Acad. Sci. USA 85:3014-3018; Armentano et al., 1990, Proc. Natl. Acad. Sci. USA 87:61416145; Huber et al., 1991, Proc. Natl. Acad. Sci. USA 88:8039-8043; Ferry et al., 1991, Proc. Natl. Acad. Sci. USA 88:8377-8381; Chowdhury et al., 1991, Science 254:1802-1805; van Beusechem. et al., 1992, Proc. Natl. Acad. Sci. USA 89:7640-19; Kay et al., 1992, Human Gene Therapy 3:641-647; Dai et al., 1992, Proc. Natl. Acad. Sci. USA 89:10892-10895; Hwu et al., 1993, J. Immunol. 150:4104-4115; U.S. Pat. Nos. 4,868,116; 4,980,286; PCT Application WO 89/07136; PCT Application WO 89/02468; PCT Application WO 89/05345; and PCT Application WO 92/07573). Recombinant retroviral vectors capable of transducing and expressing genes inserted into the genome of a cell can be produced by transfecting the recombinant retroviral genome into suitable packaging cell lines such as PA317 and Psi-CRIP (Comette et al., 1991, Human Gene Therapy 2:5-10; Cone et al., 1984, Proc. Natl. Acad. Sci. USA 81:6349). Recombinant adenoviral vectors can be used to infect a wide variety of cells and tissues in susceptible hosts (e.g., rat, hamster, dog, and chimpanzee) (Hsu et al., 1992, J. Infectious Disease, 166:769), and also have the advantage of not requiring mitotically active cells for infection.

Any viral vector capable of accepting the coding sequences for the dsRNA molecule(s) to be expressed can be used, for example vectors derived from adenovirus (AV); adeno-associated virus (AAV); retroviruses (e.g., lentiviruses (LV), Rhabdoviruses, murine leukemia virus); herpes virus, and the like. The tropism of viral vectors can be modified by pseudotyping the vectors with envelope proteins or other surface antigens from other viruses, or by substituting different viral capsid proteins, as appropriate.

For example, lentiviral vectors featured in the invention can be pseudotyped with surface proteins from vesicular stomatitis virus (VSV), rabies, Ebola, Mokola, and the like. AAV vectors featured in the invention can be made to target different cells by engineering the vectors to express different capsid protein serotypes. For example, an AAV vector expressing a serotype 2 capsid on a serotype 2 genome is called AAV 2/2. This serotype 2 capsid gene in the AAV 2/2 vector can be replaced by a serotype 5 capsid gene to produce an AAV 2/5 vector. Techniques for constructing AAV vectors which express different capsid protein serotypes are within the skill in the art; see, e.g., Rabinowitz J E et al. (2002), J Virol 76:791-801, the entire disclosure of which is herein incorporated by reference.

Selection of recombinant viral vectors suitable for use in the invention, methods for inserting nucleic acid sequences for expressing the dsRNA into the vector, and methods of delivering the viral vector to the cells of interest are within the skill in the art. See, for example, Dornburg R (1995), Gene Therap. 2: 301-310; Eglitis M A (1988), Biotechniques 6: 608-614; Miller A D (1990), Hum Gene Therap. 1: 5-14; Anderson W F (1998), Nature 392: 25-30; and Rubinson D A et al., Nat. Genet. 33: 401-406, the entire disclosures of which are herein incorporated by reference.

Viral vectors can be derived from AV and AAV. In one embodiment, the dsRNA featured in the invention is expressed as two separate, complementary single-stranded RNA molecules from a recombinant AAV vector having, for example, either the U6 or H1 RNA promoters, or the cytomegalovirus (CMV) promoter.

A suitable AV vector for expressing the dsRNA featured in the invention, a method for constructing the recombinant AV vector, and a method for delivering the vector into target cells, are described in Xia H et al. (2002), Nat. Biotech. 20: 1006-1010.

Suitable AAV vectors for expressing the dsRNA featured in the invention, methods for constructing the recombinant AV vector, and methods for delivering the vectors into target cells are described in Samulski R et al. (1987), J. Virol. 61: 3096-3101; Fisher K J et al. (1996), J. Virol, 70: 520-532; Samulski R et al. (1989), J. Virol. 63: 3822-3826; U.S. Pat. Nos. 5,252,479; 5,139,941; International Patent Application No. WO 94/13788; and International Patent Application No. WO 93/24641, the entire disclosures of which are herein incorporated by reference.

The promoter driving dsRNA expression in either a DNA plasmid or viral vector featured in the invention may be a eukaryotic RNA polymerase I (e.g., ribosomal RNA promoter), RNA polymerase II (e.g., CMV early promoter or actin promoter or U1 snRNA promoter) or generally RNA polymerase III promoter (e.g., U6 snRNA or 7SK RNA promoter) or a prokaryotic promoter, for example the T7 promoter, provided the expression plasmid also encodes T7 RNA polymerase required for transcription from a T7 promoter. The promoter can also direct transgene expression to the pancreas (see, e.g., the insulin regulatory sequence for pancreas (Bucchini et al., 1986, Proc. Natl. Acad. Sci. USA 83:2511-2515)).

In addition, expression of the transgene can be precisely regulated, for example, by using an inducible regulatory sequence and expression systems such as a regulatory sequence that is sensitive to certain physiological regulators, e.g., circulating glucose levels, or hormones (Docherty et al., 1994, FASEB J. 8:20-24). Such inducible expression systems, suitable for the control of transgene expression in cells or in mammals include regulation by ecdysone, by estrogen, progesterone, tetracycline, chemical inducers of dimerization, and isopropyl-beta-D1-thiogalactopyranoside (EPTG). A person skilled in the art would be able to choose the appropriate regulatory/promoter sequence based on the intended use of the dsRNA transgene.

Generally, recombinant vectors capable of expressing dsRNA molecules are delivered as described below, and persist in target cells. Alternatively, viral vectors can be used that provide for transient expression of dsRNA molecules. Such vectors can be repeatedly administered as necessary. Once expressed, the dsRNAs bind to target RNA and modulate its function or expression. Delivery of dsRNA expressing vectors can be systemic, such as by intravenous or intramuscular administration, by administration to target cells ex-planted from the patient followed by reintroduction into the patient, or by any other means that allows for introduction into a desired target cell.

dsRNA expression DNA plasmids are typically transfected into target cells as a complex with cationic lipid carriers (e.g., Oligofectamine) or non-cationic lipid-based carriers (e.g., Transit-TKO™). Multiple lipid transfections for dsRNA-mediated knockdowns targeting different regions of a single APOC3 gene or multiple APOC3 genes over a period of a week or more are also contemplated by the invention. Successful introduction of vectors into host cells can be monitored using various known methods. For example, transient transfection can be signaled with a reporter, such as a fluorescent marker, such as Green Fluorescent Protein (GFP). Stable transfection of cells ex vivo can be ensured using markers that provide the transfected cell with resistance to specific environmental factors (e.g., antibiotics and drugs), such as hygromycin B resistance.

APOC3 specific dsRNA molecules can also be inserted into vectors and used as gene therapy vectors for human patients. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can include a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

Pharmaceutical Compositions Containing dsRNA

In one embodiment, the invention provides pharmaceutical compositions containing a dsRNA, as described herein, and a pharmaceutically acceptable carrier. The pharmaceutical composition containing the dsRNA is useful for treating a disease or disorder associated with the expression or activity of an APOC3 gene, such as pathological processes mediated by APOC3 expression. Such pharmaceutical compositions are formulated based on the mode of delivery.

The pharmaceutical compositions featured herein are administered in dosages sufficient to inhibit expression of an APOC3 gene.

In general, a suitable dose of dsRNA will be in the range of 0.01 to 200.0 milligrams per kilogram body weight of the recipient per day, generally in the range of 1 to 50 mg per kilogram body weight per day.

Subjects can be administered a therapeutic amount of dsRNA, such as about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg, 0.1 mg/kg, 0.15 mg/kg, 0.2 mg/kg, 0.25 mg/kg, 0.3 mg/kg, 0.35 mg/kg, 0.4 mg/kg, 0.45 mg/kg, 0.5 mg/kg, 0.55 mg/kg, 0.6 mg/kg, 0.65 mg/kg, 0.7 mg/kg, 0.75 mg/kg, 0.8 mg/kg, 0.85 mg/kg, 0.9 mg/kg, 0.95 mg/kg, 1.0 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg, 2.1 mg/kg, 2.2 mg/kg, 2.3 mg/kg, 2.4 mg/kg, 2.5 mg/kg dsRNA, 2.6 mg/kg dsRNA, 2.7 mg/kg dsRNA, 2.8 mg/kg dsRNA, 2.9 mg/kg dsRNA, 3.0 mg/kg dsRNA, 3.1 mg/kg dsRNA, 3.2 mg/kg dsRNA, 3.3 mg/kg dsRNA, 3.4 mg/kg dsRNA, 3.5 mg/kg dsRNA, 3.6 mg/kg dsRNA, 3.7 mg/kg dsRNA, 3.8 mg/kg dsRNA, 3.9 mg/kg dsRNA, 4.0 mg/kg dsRNA, 4.1 mg/kg dsRNA, 4.2 mg/kg dsRNA, 4.3 mg/kg dsRNA, 4.4 mg/kg dsRNA, 4.5 mg/kg dsRNA, 4.6 mg/kg dsRNA, 4.7 mg/kg dsRNA, 4.8 mg/kg dsRNA, 4.9 mg/kg dsRNA, 5.0 mg/kg dsRNA, 5.1 mg/kg dsRNA, 5.2 mg/kg dsRNA, 5.3 mg/kg dsRNA, 5.4 mg/kg dsRNA, 5.5 mg/kg dsRNA, 5.6 mg/kg dsRNA, 5.7 mg/kg dsRNA, 5.8 mg/kg dsRNA, 5.9 mg/kg dsRNA, 6.0 mg/kg dsRNA, 6.1 mg/kg dsRNA, 6.2 mg/kg dsRNA, 6.3 mg/kg dsRNA, 6.4 mg/kg dsRNA, 6.5 mg/kg dsRNA, 6.6 mg/kg dsRNA, 6.7 mg/kg dsRNA, 6.8 mg/kg dsRNA, 6.9 mg/kg dsRNA, 7.0 mg/kg dsRNA, 7.1 mg/kg dsRNA, 7.2 mg/kg dsRNA, 7.3 mg/kg dsRNA, 7.4 mg/kg dsRNA, 7.5 mg/kg dsRNA, 7.6 mg/kg dsRNA, 7.7 mg/kg dsRNA, 7.8 mg/kg dsRNA, 7.9 mg/kg dsRNA, 8.0 mg/kg dsRNA, 8.1 mg/kg dsRNA, 8.2 mg/kg dsRNA, 8.3 mg/kg dsRNA, 8.4 mg/kg dsRNA, 8.5 mg/kg dsRNA, 8.6 mg/kg dsRNA, 8.7 mg/kg dsRNA, 8.8 mg/kg dsRNA, 8.9 mg/kg dsRNA, 9.0 mg/kg dsRNA, 9.1 mg/kg dsRNA, 9.2 mg/kg dsRNA, 9.3 mg/kg dsRNA, 9.4 mg/kg dsRNA, 9.5 mg/kg dsRNA, 9.6 mg/kg dsRNA, 9.7 mg/kg dsRNA, 9.8 mg/kg dsRNA, 9.9 mg/kg dsRNA, 9.0 mg/kg dsRNA, 10 mg/kg dsRNA, 15 mg/kg dsRNA, 20 mg/kg dsRNA, 25 mg/kg dsRNA, 30 mg/kg dsRNA, 35 mg/kg dsRNA, 40 mg/kg dsRNA, 45 mg/kg dsRNA, or about 50 mg/kg dsRNA. Values and ranges intermediate to the recited values are also intended to be part of this invention.

The pharmaceutical composition may be administered once daily or the dsRNA may be administered as two, three, or more sub-doses at appropriate intervals throughout the day or even using continuous infusion or delivery through a controlled release formulation. In that case, the dsRNA contained in each sub-dose must be correspondingly smaller in order to achieve the total daily dosage. The dosage unit can also be compounded for delivery over several days, e.g., using a conventional sustained release formulation which provides sustained release of the dsRNA over a several day period. Sustained release formulations are well known in the art and are particularly useful for delivery of agents at a particular site, such as could be used with the agents of the present invention. In this embodiment, the dosage unit contains a corresponding multiple of the daily dose.

The effect of a single dose on APOC3 levels is long lasting, such that subsequent doses are administered at not more than 3, 4, or 5 day intervals, or at not more than 1, 2, 3, or 4 week intervals, or at not more than 5, 6, 7, 8, 9, or 10 week intervals.

The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a composition can include a single treatment or a series of treatments. Estimates of effective dosages and in vivo half-lives for the individual dsRNAs encompassed by the invention can be made using conventional methodologies or on the basis of in vivo testing using an appropriate animal model, as described elsewhere herein.

Advances in mouse genetics have generated a number of mouse models for the study of various human diseases, such as pathological processes mediated by APOC3 expression. Such models are used for in vivo testing of dsRNA, as well as for determining a therapeutically effective dose. A suitable mouse model is, for example, a mouse containing a plasmid expressing human APOC3. Another suitable mouse model is a transgenic mouse carrying a transgene that expresses human APOC3.

The data obtained from cell culture assays and animal studies can be used in formulating a range of dosage for use in humans. The dosage of compositions featured in the invention lies generally within a range of circulating concentrations that include the ED50 with little or no toxicity. The dosage may vary within this range depending upon the dosage form employed and the route of administration utilized. For any compound used in the methods featured in the invention, the therapeutically effective dose can be estimated initially from cell culture assays. A dose may be formulated in animal models to achieve a circulating plasma concentration range of the compound or, when appropriate, of the polypeptide product of a target sequence (e.g., achieving a decreased concentration of the polypeptide) that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

The dsRNAs featured in the invention can be administered in combination with other known agents effective in treatment of pathological processes mediated by target gene expression. In any event, the administering physician can adjust the amount and timing of dsRNA administration on the basis of results observed using standard measures of efficacy known in the art or described herein.

Administration

The present invention also includes pharmaceutical compositions and formulations which include the dsRNA compounds featured in the invention. The pharmaceutical compositions of the present invention may be administered in a number of ways depending upon whether local or systemic treatment is desired and upon the area to be treated. Administration may be topical (including buccal and sublingual), pulmonary, e.g., by inhalation or insufflation of powders or aerosols, including by nebulizer; intratracheal, intranasal, epidermal and transdermal, oral or parenteral. Parenteral administration includes intravenous, intraarterial, subcutaneous, intraperitoneal or intramuscular injection or infusion; or intracranial, e.g., intraparenchymal, intrathecal or intraventricular administration.

The dsRNA can be delivered in a manner to target a particular tissue.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful. Suitable topical formulations include those in which the dsRNAs featured in the invention are in admixture with a topical delivery agent such as lipids, liposomes, fatty acids, fatty acid esters, steroids, chelating agents and surfactants. Suitable lipids and liposomes include neutral (e.g., dioleoylphosphatidyl DOPE ethanolamine, dimyristoylphosphatidyl choline DMPC, distearoylphosphatidyl choline) negative (e.g., dimyristoylphosphatidyl glycerol DMPG) and cationic (e.g., dioleoyltetramethylaminopropyl DOTAP and dioleoylphosphatidyl ethanolamine DOTMA). DsRNAs featured in the invention may be encapsulated within liposomes or may form complexes thereto, in particular to cationic liposomes. Alternatively, dsRNAs may be complexed to lipids, in particular to cationic lipids. Suitable fatty acids and esters include but are not limited to arachidonic acid, oleic acid, eicosanoic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a C₁₋₁₀ alkyl ester (e.g., isopropylmyristate IPM), monoglyceride, diglyceride or pharmaceutically acceptable salt thereof. Topical formulations are described in detail in U.S. Pat. No. 6,747,014, which is incorporated herein by reference.

Liposomal Formulations

There are many organized surfactant structures besides microemulsions that have been studied and used for the formulation of drugs. These include monolayers, micelles, bilayers and vesicles. Vesicles, such as liposomes, have attracted great interest because of their specificity and the duration of action they offer from the standpoint of drug delivery. As used in the present invention, the term “liposome” means a vesicle composed of amphiphilic lipids arranged in a spherical bilayer or bilayers.

Liposomes are unilamellar or multilamellar vesicles which have a membrane formed from a lipophilic material and an aqueous interior. The aqueous portion contains the composition to be delivered. Cationic liposomes possess the advantage of being able to fuse to the cell wall. Non-cationic liposomes, although not able to fuse as efficiently with the cell wall, are taken up by macrophages in vivo.

In order to cross intact mammalian skin, lipid vesicles must pass through a series of fine pores, each with a diameter less than 50 nm, under the influence of a suitable transdermal gradient. Therefore, it is desirable to use a liposome which is highly deformable and able to pass through such fine pores.

Further advantages of liposomes include; liposomes obtained from natural phospholipids are biocompatible and biodegradable; liposomes can incorporate a wide range of water and lipid soluble drugs; liposomes can protect encapsulated drugs in their internal compartments from metabolism and degradation (Rosoff, in Pharmaceutical Dosage Forms, Lieberman, Rieger and Banker (Eds.), 1988, Marcel Dekker, Inc., New York, N.Y., volume 1, p. 245). Important considerations in the preparation of liposome formulations are the lipid surface charge, vesicle size and the aqueous volume of the liposomes.

Liposomes are useful for the transfer and delivery of active ingredients to the site of action. Because the liposomal membrane is structurally similar to biological membranes, when liposomes are applied to a tissue, the liposomes start to merge with the cellular membranes and as the merging of the liposome and cell progresses, the liposomal contents are emptied into the cell where the active agent may act.

Liposomal formulations have been the focus of extensive investigation as the mode of delivery for many drugs. There is growing evidence that for topical administration, liposomes present several advantages over other formulations. Such advantages include reduced side-effects related to high systemic absorption of the administered drug, increased accumulation of the administered drug at the desired target, and the ability to administer a wide variety of drugs, both hydrophilic and hydrophobic, into the skin.

Several reports have detailed the ability of liposomes to deliver agents including high-molecular weight DNA into the skin. Compounds including analgesics, antibodies, hormones and high-molecular weight DNAs have been administered to the skin. The majority of applications resulted in the targeting of the upper epidermis

Liposomes fall into two broad classes. Cationic liposomes are positively charged liposomes which interact with the negatively charged DNA molecules to form a stable complex. The positively charged DNA/liposome complex binds to the negatively charged cell surface and is internalized in an endosome. Due to the acidic pH within the endosome, the liposomes are ruptured, releasing their contents into the cell cytoplasm (Wang et al., Biochem. Biophys. Res. Commun., 1987, 147, 980-985).

Liposomes which are pH-sensitive or negatively-charged, entrap DNA rather than complex with it. Since both the DNA and the lipid are similarly charged, repulsion rather than complex formation occurs. Nevertheless, some DNA is entrapped within the aqueous interior of these liposomes. pH-sensitive liposomes have been used to deliver DNA encoding the thymidine kinase gene to cell monolayers in culture. Expression of the exogenous gene was detected in the target cells (Zhou et al., Journal of Controlled Release, 1992, 19, 269-274).

One major type of liposomal composition includes phospholipids other than naturally-derived phosphatidylcholine. Neutral liposome compositions, for example, can be formed from dimyristoyl phosphatidylcholine (DMPC) or dipalmitoyl phosphatidylcholine (DPPC). Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example, soybean PC, and egg PC. Another type is formed from mixtures of phospholipid and/or phosphatidylcholine and/or cholesterol.

Several studies have assessed the topical delivery of liposomal drug formulations to the skin. Application of liposomes containing interferon to guinea pig skin resulted in a reduction of skin herpes sores while delivery of interferon via other means (e.g., as a solution or as an emulsion) were ineffective (Weiner et al., Journal of Drug Targeting, 1992, 2, 405-410). Further, an additional study tested the efficacy of interferon administered as part of a liposomal formulation to the administration of interferon using an aqueous system, and concluded that the liposomal formulation was superior to aqueous administration (du Plessis et al., Antiviral Research, 1992, 18, 259-265).

Non-ionic liposomal systems have also been examined to determine their utility in the delivery of drugs to the skin, in particular systems comprising non-ionic surfactant and cholesterol. Non-ionic liposomal formulations comprising Novasome™ I (glyceryl dilaurate/cholesterol/polyoxyethylene-10-stearyl ether) and Novasome™ II (glyceryl distearate/cholesterol/polyoxyethylene-10-stearyl ether) were used to deliver cyclosporin-A into the dermis of mouse skin. Results indicated that such non-ionic liposomal systems were effective in facilitating the deposition of cyclosporin-A into different layers of the skin (Hu et al. S.T.P. Pharma. Sci., 1994, 4, 6, 466).

Liposomes also include “sterically stabilized” liposomes, a term which, as used herein, refers to liposomes comprising one or more specialized lipids that, when incorporated into liposomes, result in enhanced circulation lifetimes relative to liposomes lacking such specialized lipids. Examples of sterically stabilized liposomes are those in which part of the vesicle-forming lipid portion of the liposome (A) comprises one or more glycolipids, such as monosialoganglioside G_{M1}, or (B) is derivatized with one or more hydrophilic polymers, such as a polyethylene glycol (PEG) moiety. While not wishing to be bound by any particular theory, it is thought in the art that, at least for sterically stabilized liposomes containing gangliosides, sphingomyelin, or PEG-derivatized lipids, the enhanced circulation half-life of these sterically stabilized liposomes derives from a reduced uptake into cells of the reticuloendothelial system (RES) (Allen et al., FEBS Letters, 1987, 223, 42; Wu et al., Cancer Research, 1993, 53, 3765).

Various liposomes comprising one or more glycolipids are known in the art. Papahadjopoulos et al. (Ann. N.Y. Acad. Sci., 1987, 507, 64) reported the ability of monosialoganglioside G_{M1}, galactocerebroside sulfate and phosphatidylinositol to improve blood half-lives of liposomes. These findings were expounded upon by Gabizon et al. (Proc. Natl. Acad. Sci. U.S.A., 1988, 85, 6949). U.S. Pat. No. 4,837,028 and WO 88/04924, both to Allen et al., disclose liposomes comprising (1) sphingomyelin and (2) the ganglioside G_{M1} or a galactocerebroside sulfate ester. U.S. Pat. No. 5,543,152 (Webb et al.) discloses liposomes comprising sphingomyelin. Liposomes comprising 1,2-sn-dimyristoylphosphatidylcholine are disclosed in WO 97/13499 (Lim et al.).

Many liposomes comprising lipids derivatized with one or more hydrophilic polymers, and methods of preparation thereof, are known in the art. Sunamoto et al. (Bull. Chem. Soc. Jpn., 1980, 53, 2778) described liposomes comprising a nonionic detergent, 2C_{1215G}, that contains a PEG moiety. Illum et al. (FEBS Lett., 1984, 167, 79) noted that hydrophilic coating of polystyrene particles with polymeric glycols results in significantly enhanced blood half-lives. Synthetic phospholipids modified by the attachment of carboxylic groups of polyalkylene glycols (e.g., PEG) are described by Sears (U.S. Pat. Nos. 4,426,330 and 4,534,899). Klibanov et al. (FEBS Lett., 1990, 268, 235) described experiments demonstrating that liposomes comprising phosphatidylethanolamine (PE) derivatized with PEG or PEG stearate have significant increases in blood circulation half-lives. Blume et al. (Biochimica et Biophysica Acta, 1990, 1029, 91) extended such observations to other PEG-derivatized phospholipids, e.g., DSPE-PEG, formed from the combination of distearoylphosphatidylethanolamine (DSPE) and PEG. Liposomes having covalently bound PEG moieties on their external surface are described in European Patent No. EP 0 445 131 B1 and WO 90/04384 to Fisher. Liposome compositions containing 1-20 mole percent of PE derivatized with PEG, and methods of use thereof, are described by Woodle et al. (U.S. Pat. Nos. 5,013,556 and 5,356,633) and Martin et al. (U.S. Pat. No. 5,213,804 and European Patent No. EP 0 496 813 B1). Liposomes comprising a number of other lipid-polymer conjugates are disclosed in WO 91/05545 and U.S. Pat. No. 5,225,212 (both to Martin et al.) and in WO 94/20073 (Zalipsky et al.) Liposomes comprising PEG-modified ceramide lipids are described in WO 96/10391 (Choi et al). U.S. Pat. No. 5,540,935 (Miyazaki et al.) and U.S. Pat. No. 5,556,948 (Tagawa et al.) describe PEG-containing liposomes that can be further derivatized with functional moieties on their surfaces.

A number of liposomes comprising nucleic acids are known in the art. WO 96/40062 to Thierry et al. discloses methods for encapsulating high molecular weight nucleic acids in liposomes. U.S. Pat. No. 5,264,221 to Tagawa et al. discloses protein-bonded liposomes and asserts that the contents of such liposomes may include a dsRNA. U.S. Pat. No. 5,665,710 to Rahman et al. describes certain methods of encapsulating oligodeoxynucleotides in liposomes. WO 97/04787 to Love et al. discloses liposomes comprising dsRNAs targeted to the raf gene.

Transfersomes are yet another type of liposomes, and are highly deformable lipid aggregates which are attractive candidates for drug delivery vehicles. Transfersomes may be described as lipid droplets which are so highly deformable that they are easily able to penetrate through pores which are smaller than the droplet. Transfersomes are adaptable to the environment in which they are used, e.g., they are self-optimizing (adaptive to the shape of pores in the skin), self-repairing, frequently reach their targets without fragmenting, and often self-loading. To make transfersomes it is possible to add surface edge-activators, usually surfactants, to a standard liposomal composition. Transfersomes have been used to deliver serum albumin to the skin. The transfersome-mediated delivery of serum albumin has been shown to be as effective as subcutaneous injection of a solution containing serum albumin.

Surfactants find wide application in formulations such as emulsions (including microemulsions) and liposomes. The most common way of classifying and ranking the properties of the many different types of surfactants, both natural and synthetic, is by the use of the hydrophile/lipophile balance (HLB). The nature of the hydrophilic group (also known as the “head”) provides the most useful means for categorizing the different surfactants used in formulations (Rieger, in Pharmaceutical Dosage Forms, Marcel Dekker, Inc., New York, N.Y., 1988, p. 285).

If the surfactant molecule is not ionized, it is classified as a nonionic surfactant. Nonionic surfactants find wide application in pharmaceutical and cosmetic products and are usable over a wide range of pH values. In general their HLB values range from 2 to about 18 depending on their structure. Nonionic surfactants include nonionic esters such as ethylene glycol esters, propylene glycol esters, glyceryl esters, polyglyceryl esters, sorbitan esters, sucrose esters, and ethoxylated esters. Nonionic alkanolamides and ethers such as fatty alcohol ethoxylates, propoxylated alcohols, and ethoxylated/propoxylated block polymers are also included in this class. The polyoxyethylene surfactants are the most popular members of the nonionic surfactant class.

If the surfactant molecule carries a negative charge when it is dissolved or dispersed in water, the surfactant is classified as anionic. Anionic surfactants include carboxylates such as soaps, acyl lactylates, acyl amides of amino acids, esters of sulfuric acid such as alkyl sulfates and ethoxylated alkyl sulfates, sulfonates such as alkyl benzene sulfonates, acyl isethionates, acyl taurates and sulfosuccinates, and phosphates. The most important members of the anionic surfactant class are the alkyl sulfates and the soaps.

If the surfactant molecule carries a positive charge when it is dissolved or dispersed in water, the surfactant is classified as cationic. Cationic surfactants include quaternary ammonium salts and ethoxylated amines. The quaternary ammonium salts are the most used members of this class.

If the surfactant molecule has the ability to carry either a positive or negative charge, the surfactant is classified as amphoteric. Amphoteric surfactants include acrylic acid derivatives, substituted alkylamides, N-alkylbetaines and phosphatides.

The use of surfactants in drug products, formulations and in emulsions has been reviewed (Rieger, in Pharmaceutical Dosage Forms, Marcel Dekker, Inc., New York, N.Y., 1988, p. 285).

Nucleic Acid Lipid Particles

In one embodiment, an APOC3 dsRNA featured in the invention is fully encapsulated in the lipid formulation, e.g., to form a SPLP, pSPLP, SNALP, or other nucleic acid-lipid particle. As used herein, the term “SNALP” refers to a stable nucleic acid-lipid particle, including SPLP. As used herein, the term “SPLP” refers to a nucleic acid-lipid particle comprising plasmid DNA encapsulated within a lipid vesicle. SNALPs and SPLPs typically contain a cationic lipid, a non-cationic lipid, and a lipid that prevents aggregation of the particle (e.g., a PEG-lipid conjugate). SNALPs and SPLPs are extremely useful for systemic applications, as they exhibit extended circulation lifetimes following intravenous (i.v.) injection and accumulate at distal sites (e.g., sites physically separated from the administration site). SPLPs include “pSPLP,” which include an encapsulated condensing agent-nucleic acid complex as set forth in PCT Publication No. WO 00/03683. The particles of the present invention typically have a mean diameter of about 50 nm to about 150 nm, more typically about 60 nm to about 130 nm, more typically about 70 nm to about 110 nm, most typically about 70 nm to about 90 nm, and are substantially nontoxic. In addition, the nucleic acids when present in the nucleic acid-lipid particles of the present invention are resistant in aqueous solution to degradation with a nuclease. Nucleic acid-lipid particles and their method of preparation are disclosed in, e.g., U.S. Pat. Nos. 5,976,567; 5,981,501; 6,534,484; 6,586,410; 6,815,432; and PCT Publication No. WO 96/40964.

In one embodiment, the lipid to drug ratio (mass/mass ratio) (e.g., lipid to dsRNA ratio) will be in the range of from about 1:1 to about 50:1, from about 1:1 to about 25:1, from about 3:1 to about 15:1, from about 4:1 to about 10:1, from about 5:1 to about 9:1, or about 6:1 to about 9:1. In some embodiments the lipid to dsRNA ratio can be about 1:1, 2:1, 3:1, 4:1, 5:1, 6:1, 7:1, 8:1, 9:1, 10:1, or 11:1.

In general, the lipid-nucleic acid particle is suspended in a buffer, e.g., PBS, for administration. In one embodiment, the pH of the lipid formulated siRNA is between 6.8 and 7.8, e.g., 7.3 or 7.4. The osmolality can be, e.g., between 250 and 350 mOsm/kg, e.g., around 300, e.g., 298, 299, 300, 301, 302, 303, 304, or 305.

The cationic lipid may be, for example, N,N-dioleyl-N,N-dimethylammonium chloride (DODAC), N,N-distearyl-N,N-dimethylammonium bromide (DDAB), N—(I-(2,3-dioleoyloxy)propyl)-N,N,N-trimethylammonium chloride (DOTAP), N—(I-(2,3-dioleyloxy)propyl)-N,N,N-trimethylammonium chloride (DOTMA), N,N-dimethyl-2,3-dioleyloxy)propylamine (DODMA), 1,2-DiLinoleyloxy-N,N-dimethylaminopropane (DLinDMA), 1,2-Dilinolenyloxy-N,N-dimethylaminopropane (DLenDMA), 1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA·Cl), 1,2-Dilinoleoyl-3-trimethylaminopropane chloride salt (DLin-TAP·Cl), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanedio (DOAP), 1,2-Dilinoleyloxo-3-(2-N,N-dimethylamino)ethoxypropane (DLin-EG-DMA), 1,2-Dilinolenyloxy-N,N-dimethylaminopropane (DLinDMA), 2,2-Dilinoleyl-4-dimethylaminomethyl-[1,3]-dioxolane (DLin-K-DMA) or analogs thereof, (3aR,5s,6aS)—N,N-dimethyl-2,2-di((9Z,12Z)-octadeca-9,12-dienyl)tetrahydro-3aH-cyclopenta[d][1,3]dioxol-5-amine (ALN100), (6Z,9Z,28Z,31Z)-heptatriaconta-6,9,28,31-tetraen-19-yl 4-(dimethylamino)butanoate (MC3), 1,1′-(2-(4-(2-((2-(bis(2-hydroxydodecyl)amino)ethyl)(2-hydroxydodecyl)amino)ethyl)piperazin-1-yl)ethylazanediyl)didodecan-2-ol (C12-200 or Tech G1), or a mixture thereof. The cationic lipid may comprise from about 20 mol % to about 50 mol % or about 40 mol % of the total lipid present in the particle.

The non-cationic lipid may be an anionic lipid or a neutral lipid including, but not limited to, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-l-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-O-dimethyl PE, 18-1-trans PE, 1-stearoyl-2-oleoyl-phosphatidyethanolamine (SOPE), cholesterol, or a mixture thereof. The non-cationic lipid may be from about 5 mol % to about 90 mol %, about 10 mol %, or about 58 mol % if cholesterol is included, of the total lipid present in the particle.

The conjugated lipid that inhibits aggregation of particles may be, for example, a polyethyleneglycol (PEG)-lipid including, without limitation, a PEG-diacylglycerol (DAG), a PEG-dialkyloxypropyl (DAA), a PEG-phospholipid, a PEG-ceramide (Cer), or a mixture thereof. The PEG-DAA conjugate may be, for example, a PEG-dilauryloxypropyl (Ci₂), a PEG-dimyristyloxypropyl (Ci₄), a PEG-dipalmityloxypropyl (C1₆), or a PEG-distearyloxypropyl (C₁₈). Other examples of PEG conjugates include PEG-cDMA (N-[(methoxy poly(ethylene glycol)2000)carbamyl]-1,2-dimyristyloxlpropyl-3-amine), mPEG2000-DMG (mPEG-dimyrystylglycerol (with an average molecular weight of 2,000) and PEG-C-DOMG (R-3-[(ω-methoxy-poly(ethylene glycol)2000)carbamoyl)]-1,2-dimyristyloxlpropyl-3-amine). The conjugated lipid that prevents aggregation of particles may be from 0 mol % to about 20 mol % or about 1.0, 1.1, 1.2, 0.13, 1.4, 1.5, 1.6, 1.7, 1.8, 1.9, or 2 mol % of the total lipid present in the particle.

In some embodiments, the nucleic acid-lipid particle further includes cholesterol at, e.g., about 10 mol % to about 60 mol % or about 48 mol % of the total lipid present in the particle.

In one embodiment, the compound 2,2-Dilinoleyl-4-dimethylaminoethyl-[1,3]-dioxolane can be used to prepare lipid-siRNA nanoparticles. Synthesis of 2,2-Dilinoleyl-4-dimethylaminoethyl-[1,3]-dioxolane is described in U.S. provisional patent application No. 61/107,998 filed on Oct. 23, 2008, which is herein incorporated by reference.

For example, the lipid-siRNA particle can include 40% 2,2-Dilinoleyl-4-dimethylaminoethyl-[1,3]-dioxolane: 10% DSPC: 40% Cholesterol: 10% PEG-C-DOMG (mole percent) with a particle size of 63.0±20 nm and a 0.027 siRNA/Lipid Ratio.

In still another embodiment, the compound 1,1′-(2-(4-(2-((2-(bis(2-hydroxydodecyl)amino)ethyl)(2-hydroxydodecyl)amino)ethyl)piperazin-1-yl)ethylazanediyl)didodecan-2-ol (Tech G1) can be used to prepare lipid-siRNA particles. For example, the dsRNA can be formulated in a lipid formulation comprising Tech-G1, distearoyl phosphatidylcholine (DSPC), cholesterol and mPEG2000-DMG at a molar ratio of 50:10:38.5:1.5 at a total lipid to siRNA ratio of 7:1 (wt:wt).

In one embodiment, the lipidoid ND98·4HCl (MW 1487), Cholesterol (Sigma-Aldrich), and PEG-Ceramide C16 (Avanti Polar Lipids) can be used to prepare lipid-siRNA nanoparticles (i.e., LNP01 particles). LNP01 formulations are described, e.g., in International Application Publication No. WO 2008/042973, which is hereby incorporated by reference.

Additional exemplary formulations are described in Table A.

TABLE A

| | | cationic lipid/non-cationic lipid/cholesterol/ |
|---|---|---|
| | | PEG-lipid conjugate |
| | | Mol % ratios |
| | Cationic Lipid | Lipid:siRNA ratio |
| SNALP | DLinDMA | DLinDMA/DPPC/Cholesterol/PEG-cDMA |
| | | (57.1/7.1/34.4/1.4) |
| | | lipid:siRNA ~7:1 |
| S-XTC | XTC | XTC/DPPC/Cholesterol/PEG-cDMA |
| | | 57.1/7.1/34.4/1.4 |
| | | lipid:siRNA ~7:1 |
| LNP05 | XTC | XTC/DSPC/Cholesterol/PEG-DMG |
| | | 57.5/7.5/31.5/3.5 |
| | | lipid:siRNA ~6:1 |
| LNP06 | XTC | XTC/DSPC/Cholesterol/PEG-DMG |
| | | 57.5/7.5/31.5/3.5 |
| | | lipid:siRNA ~11:1 |
| LNP07 | XTC | XTC/DSPC/Cholesterol/PEG-DMG |
| | | 60/7.5/31/1.5, |
| | | lipid:siRNA ~6:1 |
| LNP08 | XTC | XTC/DSPC/Cholesterol/PEG-DMG |
| | | 60/7.5/31/1.5, |
| | | lipid:siRNA ~11:1 |
| LNP09 | XTC | XTC/DSPC/Cholesterol/PEG-DMG |
| | | 50/10/38.5/1.5 |
| | | Lipid:siRNA 10:1 |
| LNP10 | ALN100 | ALN100/DSPC/Cholesterol/PEG-DMG |
| | | 50/10/38.5/1.5 |
| | | Lipid:siRNA 10:1 |
| LNP11 | MC3 | MC-3/DSPC/Cholesterol/PEG-DMG |
| | | 50/10/38.5/1.5 |
| | | Lipid:siRNA 10:1 |
| LNP12 | C12-200 | C12-200/DSPC/Cholesterol/PEG-DMG |
| | | 50/10/38.5/1.5 |
| | | Lipid:siRNA 10:1 |
| LNP13 | XTC | XTC/DSPC/Chol/PEG-DMG |
| | | 50/10/38.5/1.5 |
| | | Lipid:siRNA: 33:1 |
| LNP14 | MC3 | MC3/DSPC/Chol/PEG-DMG |
| | | 40/15/40/5 |
| | | Lipid:siRNA: 11:1 |
| LNP15 | MC3 | MC3/DSPC/Chol/PEG-DSG/Ga1NAc- |
| | | PEG-DSG |
| | | 50/10/35/4.5/0.5 |
| | | Lipid:siRNA: 11:1 |
| LNP16 | MC3 | MC3/DSPC/Chol/PEG-DMG |
| | | 50/10/38.5/1.5 |
| | | Lipid:siRNA: 7:1 |
| LNP17 | MC3 | MC3/DSPC/Chol/PEG-DSG |
| | | 50/10/38.5/1.5 |
| | | Lipid:siRNA: 10:1 |
| LNP18 | MC3 | MC3/DSPC/Chol/PEG-DMG |
| | | 50/10/38.5/1.5 |
| | | Lipid:siRNA: 12:1 |
| LNP19 | MC3 | MC3/DSPC/Chol/PEG-DMG |
| | | 50/10/35/5 |
| | | Lipid:siRNA: 8:1 |
| LNP20 | MC3 | MC3/DSPC/Chol/PEG-DPG |
| | | 50/10/38.5/1.5 |
| | | Lipid:siRNA: 10:1 |
| LNP21 | C12-200 | C12-200/DSPC/Chol/PEG-DSG |
| | | 50/10/38.5/1.5 |
| | | Lipid:siRNA: 7:1 |
| LNP22 | XTC | XTC/DSPC/Chol/PEG-DSG |
| | | 50/10/38.5/1.5 |
| | | Lipid:siRNA: 10:1 |

SNALP (1,2-Dilinolenyloxy-N,N-dimethylaminopropane (DLinDMA)) comprising formulations are described in International Publication No. WO2009/127060, filed Apr. 15, 2009, which is hereby incorporated by reference.

XTC comprising formulations are described, e.g., in U.S. Provisional Ser. No. 61/148,366, filed Jan. 29, 2009; U.S. Provisional Ser. No. 61/156,851, filed Mar. 2, 2009; U.S. Provisional Serial No. filed Jun. 10, 2009; U.S. Provisional Ser. No. 61/228,373, filed Jul. 24, 2009; U.S. Provisional Ser. No. 61/239,686, filed Sep. 3, 2009, and International Application No. PCT/US2010/022614, filed Jan. 29, 2010, which are hereby incorporated by reference.

MC3 comprising formulations are described, e.g., in U.S. Provisional Ser. No. 61/244,834, filed Sep. 22, 2009, U.S. Provisional Ser. No. 61/185,800, filed Jun. 10, 2009, and International Application No. PCT/US10/28224, filed Jun. 10, 2010, which are hereby incorporated by reference.

ALNY-100 comprising formulations are described, e.g., International patent application number PCT/US09/63933, filed on Nov. 10, 2009, which is hereby incorporated by reference.

C12-200 comprising formulations are described in U.S. Provisional Ser. No. 61/175,770, filed May 5, 2009 and International Application No. PCT/US10/33777, filed May 5, 2010, which are hereby incorporated by reference.

Formulations prepared by either the standard or extrusion-free method can be characterized in similar manners. For example, formulations are typically characterized by visual inspection. They should be whitish translucent solutions free from aggregates or sediment. Particle size and particle size distribution of lipid-nanoparticles can be measured by light scattering using, for example, a Malvern Zetasizer Nano ZS (Malvern, USA). Particles should be about 20-300 nm, such as 40-100 nm in size. The particle size distribution should be unimodal. The total siRNA concentration in the formulation, as well as the entrapped fraction, is estimated using a dye exclusion assay. A sample of the formulated siRNA can be incubated with an RNA-binding dye, such as Ribogreen (Molecular Probes) in the presence or absence of a formulation disrupting surfactant, e.g., 0.5% Triton-X100. The total siRNA in the formulation can be determined by the signal from the sample containing the surfactant, relative to a standard curve. The entrapped fraction is determined by subtracting the “free” siRNA content (as measured by the signal in the absence of surfactant) from the total siRNA content. Percent entrapped siRNA is typically >85%. For SNALP formulation, the particle size is at least 30 nm, at least 40 nm, at least 50 nm, at least 60 nm, at least 70 nm, at least 80 nm, at least 90 nm, at least 100 nm, at least 110 nm, and at least 120 nm. The suitable range is typically about at least 50 nm to about at least 110 nm, about at least 60 nm to about at least 100 nm, or about at least 80 nm to about at least 90 nm.

Compositions and formulations for oral administration include powders or granules, microparticulates, nanoparticulates, suspensions or solutions in water or non-aqueous media, capsules, gel capsules, sachets, tablets or minitablets. Thickeners, flavoring agents, diluents, emulsifiers, dispersing aids or binders may be desirable. In some embodiments, oral formulations are those in which dsRNAs featured in the invention are administered in conjunction with one or more penetration enhancers surfactants and chelators. Suitable surfactants include fatty acids and/or esters or salts thereof, bile acids and/or salts thereof. Suitable bile acids/salts include chenodeoxycholic acid (CDCA) and ursodeoxychenodeoxycholic acid (UDCA), cholic acid, dehydrocholic acid, deoxycholic acid, glucholic acid, glycholic acid, glycodeoxycholic acid, taurocholic acid, taurodeoxycholic acid, sodium tauro-24,25-dihydro-fusidate and sodium glycodihydrofusidate. Suitable fatty acids include arachidonic acid, undecanoic acid, oleic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a monoglyceride, a diglyceride or a pharmaceutically acceptable salt thereof (e.g., sodium). In some embodiments, combinations of penetration enhancers are used, for example, fatty acids/salts in combination with bile acids/salts. One exemplary combination is the sodium salt of lauric acid, capric acid and UDCA. Further penetration enhancers include polyoxyethylene-9-lauryl ether, polyoxyethylene-20-cetyl ether. DsRNAs featured in the invention may be delivered orally, in granular form including sprayed dried particles, or complexed to form micro or nanoparticles. dsRNA complexing agents include poly-amino acids; polyimines; polyacrylates; polyalkylacrylates, polyoxethanes, polyalkylcyanoacrylates; cationized gelatins, albumins, starches, acrylates, polyethyleneglycols (PEG) and starches; polyalkylcyanoacrylates; DEAE-derivatized polyimines, pullulans, celluloses and starches. Suitable complexing agents include chitosan, N-trimethylchitosan, poly-L-lysine, polyhistidine, polyornithine, polyspermines, protamine, polyvinylpyridine, polythiodiethylaminomethylethylene P(TDAE), polyaminostyrene (e.g., p-amino), poly(methylcyanoacrylate), poly(ethylcyanoacrylate), poly(butylcyanoacrylate), poly(isobutylcyanoacrylate), poly(isohexylcynaoacrylate), DEAE-methacrylate, DEAE-hexylacrylate, DEAE-acrylamide, DEAE-albumin and DEAE-dextran, polymethylacrylate, polyhexylacrylate, poly(D,L-lactic acid), poly(DL-lactic-co-glycolic acid (PLGA), alginate, and polyethyleneglycol (PEG). Oral formulations for dsRNAs and their preparation are described in detail in U.S. Pat. No. 6,887,906, US Pub. No. 20030027780, and U.S. Pat. No. 6,747,014, each of which is incorporated herein by reference.

Compositions and formulations for parenteral, intraparenchymal (into the brain), intrathecal, intraventricular or intrahepatic administration may include sterile aqueous solutions which may also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids. Particularly preferred are formulations that target the liver when treating hepatic disorders such as hepatic carcinoma.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, gel capsules, liquid syrups, soft gels, suppositories, and enemas. The compositions of the present invention may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers.

Emulsions

The compositions of the present invention may be prepared and formulated as emulsions. Emulsions are typically heterogeneous systems of one liquid dispersed in another in the form of droplets usually exceeding 0.1 m in diameter (Idson, in Pharmaceutical Dosage Forms, Lieberman, Rieger and Banker (Eds.), 1988, Marcel Dekker, Inc., New York, N.Y., volume 1, p. 199; Rosoff, in Pharmaceutical Dosage Forms, Lieberman, Rieger and Banker (Eds.), 1988, Marcel Dekker, Inc., New York, N.Y., Volume 1, p. 245; Block in Pharmaceutical Dosage Forms, Lieberman, Rieger and Banker (Eds.), 1988, Marcel Dekker, Inc., New York, N.Y., volume 2, p. 335; Higuchi et al., in Remington's Pharmaceutical Sciences, Mack Publishing Co., Easton, Pa., 1985, p. 301). Emulsions are often biphasic systems comprising two immiscible liquid phases intimately mixed and dispersed with each other. In general, emulsions may be of either the water-in-oil (w/o) or the oil-in-water (o/w) variety. When an aqueous phase is finely divided into and dispersed as minute droplets into a bulk oily phase, the resulting composition is called a water-in-oil (w/o) emulsion. Alternatively, when an oily phase is finely divided into and dispersed as minute droplets into a bulk aqueous phase, the resulting composition is called an oil-in-water (o/w) emulsion. Emulsions may contain additional components in addition to the dispersed phases, and the active drug which may be present as a solution in either the aqueous phase, oily phase or itself as a separate phase. Pharmaceutical excipients such as emulsifiers, stabilizers, dyes, and anti-oxidants may also be present in emulsions as needed. Pharmaceutical emulsions may also be multiple emulsions that are comprised of more than two phases such as, for example, in the case of oil-in-water-in-oil (o/w/o) and water-in-oil-in-water (w/o/w) emulsions. Such complex formulations often provide certain advantages that simple binary emulsions do not. Multiple emulsions in which individual oil droplets of an o/w emulsion enclose small water droplets constitute a w/o/w emulsion. Likewise a system of oil droplets enclosed in globules of water stabilized in an oily continuous phase provides an o/w/o emulsion.

Emulsions are characterized by little or no thermodynamic stability. Often, the dispersed or discontinuous phase of the emulsion is well dispersed into the external or continuous phase and maintained in this form through the means of emulsifiers or the viscosity of the formulation. Either of the phases of the emulsion may be a semisolid or a solid, as is the case of emulsion-style ointment bases and creams. Other means of stabilizing emulsions entail the use of emulsifiers that may be incorporated into either phase of the emulsion. Emulsifiers may broadly be classified into four categories: synthetic surfactants, naturally occurring emulsifiers, absorption bases, and finely dispersed solids (Idson, in Pharmaceutical Dosage Forms, Lieberman, Rieger and Banker (Eds.), 1988, Marcel Dekker, Inc., New York, N.Y., volume 1, p. 199).

Synthetic surfactants, also known as surface active agents, have found wide applicability in the formulation of emulsions and have been reviewed in the literature (Rieger, in Pharmaceutical Dosage Forms, Lieberman, Rieger and Banker (Eds.), 1988, Marcel Dekker, Inc., New York, N.Y., volume 1, p. 285; Idson, in Pharmaceutical Dosage Forms, Lieberman, Rieger and Banker (Eds.), Marcel Dekker, Inc., New York, N.Y., 1988, volume 1, p. 199). Surfactants are typically amphiphilic and comprise a hydrophilic and a hydrophobic portion. The ratio of the hydrophilic to the hydrophobic nature of the surfactant has been termed the hydrophile/lipophile balance (HLB) and is a valuable tool in categorizing and selecting surfactants in the preparation of formulations. Surfactants may be classified into different classes based on the nature of the hydrophilic group: nonionic, anionic, cationic and amphoteric (Rieger, in Pharmaceutical Dosage Forms, Lieberman, Rieger and Banker (Eds.), 1988, Marcel Dekker, Inc., New York, N.Y., volume 1, p. 285).

Naturally occurring emulsifiers used in emulsion formulations include lanolin, beeswax, phosphatides, lecithin and acacia. Absorption bases possess hydrophilic properties such that they can soak up water to form w/o emulsions yet retain their semisolid consistencies, such as anhydrous lanolin and hydrophilic petrolatum. Finely divided solids have also been used as good emulsifiers especially in combination with surfactants and in viscous preparations. These include polar inorganic solids, such as heavy metal hydroxides, non-swelling clays such as bentonite, attapulgite, hectorite, kaolin, montmorillonite, colloidal aluminum silicate and colloidal magnesium aluminum silicate, pigments and nonpolar solids such as carbon or glyceryl tristearate.

A large variety of non-emulsifying materials are also included in emulsion formulations and contribute to the properties of emulsions. These include fats, oils, waxes, fatty acids, fatty alcohols, fatty esters, humectants, hydrophilic colloids, preservatives and antioxidants (Block, in Pharmaceutical Dosage Forms, Lieberman, Rieger and Banker (Eds.), 1988, Marcel Dekker, Inc., New York, N.Y., volume 1, p. 335; Idson, in Pharmaceutical Dosage Forms, Lieberman, Rieger and Banker (Eds.), 1988, Marcel Dekker, Inc., New York, N.Y., volume 1, p. 199).

Hydrophilic colloids or hydrocolloids include naturally occurring gums and synthetic polymers such as polysaccharides (for example, acacia, agar, alginic acid, carrageenan, guar gum, karaya gum, and tragacanth), cellulose derivatives (for example, carboxymethylcellulose and carboxypropylcellulose), and synthetic polymers (for example, carbomers, cellulose ethers, and carboxyvinyl polymers). These disperse or swell in water to form colloidal solutions that stabilize emulsions by forming strong interfacial films around the dispersed-phase droplets and by increasing the viscosity of the external phase.

Since emulsions often contain a number of ingredients such as carbohydrates, proteins, sterols and phosphatides that may readily support the growth of microbes, these formulations often incorporate preservatives. Commonly used preservatives included in emulsion formulations include methyl paraben, propyl paraben, quaternary ammonium salts, benzalkonium chloride, esters of p-hydroxybenzoic acid, and boric acid. Antioxidants are also commonly added to emulsion formulations to prevent deterioration of the formulation. Antioxidants used may be free radical scavengers such as tocopherols, alkyl gallates, butylated hydroxyanisole, butylated hydroxytoluene, or reducing agents such as ascorbic acid and sodium metabisulfite, and antioxidant synergists such as citric acid, tartaric acid, and lecithin.

The application of emulsion formulations via dermatological, oral and parenteral routes and methods for their manufacture have been reviewed in the literature (Idson, in Pharmaceutical Dosage Forms, Lieberman, Rieger and Banker (Eds.), 1988, Marcel Dekker, Inc., New York, N.Y., volume 1, p. 199). Emulsion formulations for oral delivery have been very widely used because of ease of formulation, as well as efficacy from an absorption and bioavailability standpoint (Rosoff, in Pharmaceutical Dosage Forms, Lieberman, Rieger and Banker (Eds.), 1988, Marcel Dekker, Inc., New York, N.Y., volume 1, p. 245; Idson, in Pharmaceutical Dosage Forms, Lieberman, Rieger and Banker (Eds.), 1988, Marcel Dekker, Inc., New York, N.Y., volume 1, p. 199). Mineral-oil base laxatives, oil-soluble vitamins and high fat nutritive preparations are among the materials that have commonly been administered orally as o/w emulsions.

In one embodiment of the present invention, the compositions of dsRNAs and nucleic acids are formulated as microemulsions. A microemulsion may be defined as a system of water, oil and amphiphile which is a single optically isotropic and thermodynamically stable liquid solution (Rosoff, in Pharmaceutical Dosage Forms, Lieberman, Rieger and Banker (Eds.), 1988, Marcel Dekker, Inc., New York, N.Y., volume 1, p. 245). Typically microemulsions are systems that are prepared by first dispersing an oil in an aqueous surfactant solution and then adding a sufficient amount of a fourth component, generally an intermediate chain-length alcohol to form a transparent system. Therefore, microemulsions have also been described as thermodynamically stable, isotropically clear dispersions of two immiscible liquids that are stabilized by interfacial films of surface-active molecules (Leung and Shah, in: Controlled Release of Drugs: Polymers and Aggregate Systems, Rosoff, M., Ed., 1989, VCH Publishers, New York, pages 185-215). Microemulsions commonly are prepared via a combination of three to five components that include oil, water, surfactant, cosurfactant and electrolyte. Whether the microemulsion is of the water-in-oil (w/o) or an oil-in-water (o/w) type is dependent on the properties of the oil and surfactant used and on the structure and geometric packing of the polar heads and hydrocarbon tails of the surfactant molecules (Schott, in Remington's Pharmaceutical Sciences, Mack Publishing Co., Easton, Pa., 1985, p. 271).

The phenomenological approach utilizing phase diagrams has been extensively studied and has yielded a comprehensive knowledge, to one skilled in the art, of how to formulate microemulsions (Rosoff, in Pharmaceutical Dosage Forms, Lieberman, Rieger and Banker (Eds.), 1988, Marcel Dekker, Inc., New York, N.Y., volume 1, p. 245; Block, in Pharmaceutical Dosage Forms, Lieberman, Rieger and Banker (Eds.), 1988, Marcel Dekker, Inc., New York, N.Y., volume 1, p. 335). Compared to conventional emulsions, microemulsions offer the advantage of solubilizing water-insoluble drugs in a formulation of thermodynamically stable droplets that are formed spontaneously.

Surfactants used in the preparation of microemulsions include, but are not limited to, ionic surfactants, non-ionic surfactants, Brij 96, polyoxyethylene oleyl ethers, polyglycerol fatty acid esters, tetraglycerol monolaurate (ML310), tetraglycerol monooleate (MO310), hexaglycerol monooleate (PO310), hexaglycerol pentaoleate (PO500), decaglycerol monocaprate (MCA750), decaglycerol monooleate (MO750), decaglycerol sequioleate (SO750), decaglycerol decaoleate (DAO750), alone or in combination with cosurfactants. The cosurfactant, usually a short-chain alcohol such as ethanol, 1-propanol, and 1-butanol, serves to increase the interfacial fluidity by penetrating into the surfactant film and consequently creating a disordered film because of the void space generated among surfactant molecules. Microemulsions may, however, be prepared without the use of cosurfactants and alcohol-free self-emulsifying microemulsion systems are known in the art. The aqueous phase may typically be, but is not limited to, water, an aqueous solution of the drug, glycerol, PEG300, PEG400, polyglycerols, propylene glycols, and derivatives of ethylene glycol. The oil phase may include, but is not limited to, materials such as Captex 300, Captex 355, Capmul MCM, fatty acid esters, medium chain (C8-C12) mono, di, and tri-glycerides, polyoxyethylated glyceryl fatty acid esters, fatty alcohols, polyglycolized glycerides, saturated polyglycolized C8-C10 glycerides, vegetable oils and silicone oil.

Microemulsions are particularly of interest from the standpoint of drug solubilization and the enhanced absorption of drugs. Lipid based microemulsions (both o/w and w/o) have been proposed to enhance the oral bioavailability of drugs, including peptides (Constantinides et al., Pharmaceutical Research, 1994, 11, 1385-1390; Ritschel, Meth. Find. Exp. Clin. Pharmacol., 1993, 13, 205). Microemulsions afford advantages of improved drug solubilization, protection of drug from enzymatic hydrolysis, possible enhancement of drug absorption due to surfactant-induced alterations in membrane fluidity and permeability, ease of preparation, ease of oral administration over solid dosage forms, improved clinical potency, and decreased toxicity (Constantinides et al., Pharmaceutical Research, 1994, 11, 1385; Ho et al., J. Pharm. Sci., 1996, 85, 138-143). Often microemulsions may form spontaneously when their components are brought together at ambient temperature. This may be particularly advantageous when formulating thermolabile drugs, peptides or dsRNAs. Microemulsions have also been effective in the transdermal delivery of active components in both cosmetic and pharmaceutical applications. It is expected that the microemulsion compositions and formulations of the present invention will facilitate the increased systemic absorption of dsRNAs and nucleic acids from the gastrointestinal tract, as well as improve the local cellular uptake of dsRNAs and nucleic acids.

Microemulsions of the present invention may also contain additional components and additives such as sorbitan monostearate (Grill 3), Labrasol, and penetration enhancers to improve the properties of the formulation and to enhance the absorption of the dsRNAs and nucleic acids of the present invention. Penetration enhancers used in the microemulsions of the present invention may be classified as belonging to one of five broad categories—surfactants, fatty acids, bile salts, chelating agents, and non-chelating non-surfactants (Lee et al., Critical Reviews in Therapeutic Drug Carrier Systems, 1991, p. 92). Each of these classes has been discussed above.

Penetration Enhancers

In one embodiment, the present invention employs various penetration enhancers to effect the efficient delivery of nucleic acids, particularly dsRNAs, to the skin of animals. Most drugs are present in solution in both ionized and nonionized forms. However, usually only lipid soluble or lipophilic drugs readily cross cell membranes. It has been discovered that even non-lipophilic drugs may cross cell membranes if the membrane to be crossed is treated with a penetration enhancer. In addition to aiding the diffusion of non-lipophilic drugs across cell membranes, penetration enhancers also enhance the permeability of lipophilic drugs.

Penetration enhancers may be classified as belonging to one of five broad categories, i.e., surfactants, fatty acids, bile salts, chelating agents, and non-chelating non-surfactants (Lee et al., Critical Reviews in Therapeutic Drug Carrier Systems, 1991, p. 92). Each of the above mentioned classes of penetration enhancers are described below in greater detail.

Surfactants: In connection with the present invention, surfactants (or “surface-active agents”) are chemical entities which, when dissolved in an aqueous solution, reduce the surface tension of the solution or the interfacial tension between the aqueous solution and another liquid, with the result that absorption of dsRNAs through the mucosa is enhanced. In addition to bile salts and fatty acids, these penetration enhancers include, for example, sodium lauryl sulfate, polyoxyethylene-9-lauryl ether and polyoxyethylene-20-cetyl ether) (Lee et al., Critical Reviews in Therapeutic Drug Carrier Systems, 1991, p. 92); and perfluorochemical emulsions, such as FC-43. Takahashi et al., J. Pharm. Pharmacol., 1988, 40, 252).

Fatty acids: Various fatty acids and their derivatives which act as penetration enhancers include, for example, oleic acid, lauric acid, capric acid (n-decanoic acid), myristic acid, palmitic acid, stearic acid, linoleic acid, linolenic acid, dicaprate, tricaprate, monoolein (1-monooleoyl-rac-glycerol), dilaurin, caprylic acid, arachidonic acid, glycerol 1-monocaprate, 1-dodecylazacycloheptan-2-one, acylcarnitines, acylcholines, C.sub.1-10 alkyl esters thereof (e.g., methyl, isopropyl and t-butyl), and mono- and di-glycerides thereof (i.e., oleate, laurate, caprate, myristate, palmitate, stearate, linoleate, etc.) (Lee et al., Critical Reviews in Therapeutic Drug Carrier Systems, 1991, p.92; Muranishi, Critical Reviews in Therapeutic Drug Carrier Systems, 1990, 7, 1-33; El Hariri et al., J. Pharm. Pharmacol., 1992, 44, 651-654).

Bile salts: The physiological role of bile includes the facilitation of dispersion and absorption of lipids and fat-soluble vitamins (Brunton, Chapter 38 in: Goodman & Gilman's The Pharmacological Basis of Therapeutics, 9th Ed., Hardman et al. Eds., McGraw-Hill, New York, 1996, pp. 934-935). Various natural bile salts, and their synthetic derivatives, act as penetration enhancers. Thus the term “bile salts” includes any of the naturally occurring components of bile as well as any of their synthetic derivatives. Suitable bile salts include, for example, cholic acid (or its pharmaceutically acceptable sodium salt, sodium cholate), dehydrocholic acid (sodium dehydrocholate), deoxycholic acid (sodium deoxycholate), glucholic acid (sodium glucholate), glycholic acid (sodium glycocholate), glycodeoxycholic acid (sodium glycodeoxycholate), taurocholic acid (sodium taurocholate), taurodeoxycholic acid (sodium taurodeoxycholate), chenodeoxycholic acid (sodium chenodeoxycholate), ursodeoxycholic acid (UDCA), sodium tauro-24,25-dihydro-fusidate (STDHF), sodium glycodihydrofusidate and polyoxyethylene-9-lauryl ether (POE) (Lee et al., Critical Reviews in Therapeutic Drug Carrier Systems, 1991, page 92; Swinyard, Chapter 39 In: Remington's Pharmaceutical Sciences, 18th Ed., Gennaro, ed., Mack Publishing Co., Easton, Pa., 1990, pages 782-783; Muranishi, Critical Reviews in Therapeutic Drug Carrier Systems, 1990, 7, 1-33; Yamamoto et al., J. Pharm. Exp. Ther., 1992, 263, 25; Yamashita et al., J. Pharm. Sci., 1990, 79, 579-583).

Chelating Agents: Chelating agents, as used in connection with the present invention, can be defined as compounds that remove metallic ions from solution by forming complexes therewith, with the result that absorption of dsRNAs through the mucosa is enhanced. With regards to their use as penetration enhancers in the present invention, chelating agents have the added advantage of also serving as DNase inhibitors, as most characterized DNA nucleases require a divalent metal ion for catalysis and are thus inhibited by chelating agents (Jarrett, J. Chromatogr., 1993, 618, 315-339). Suitable chelating agents include but are not limited to disodium ethylenediaminetetraacetate (EDTA), citric acid, salicylates (e.g., sodium salicylate, 5-methoxysalicylate and homovanilate), N-acyl derivatives of collagen, laureth-9 and N-amino acyl derivatives of beta-diketones (enamines)(Lee et al., Critical Reviews in Therapeutic Drug Carrier Systems, 1991, page 92; Muranishi, Critical Reviews in Therapeutic Drug Carrier Systems, 1990, 7, 1-33; Buur et al., J. Control Rel., 1990, 14, 43-51).

Non-chelating non-surfactants: As used herein, non-chelating non-surfactant penetration enhancing compounds can be defined as compounds that demonstrate insignificant activity as chelating agents or as surfactants but that nonetheless enhance absorption of dsRNAs through the alimentary mucosa (Muranishi, Critical Reviews in Therapeutic Drug Carrier Systems, 1990, 7, 1-33). This class of penetration enhancers include, for example, unsaturated cyclic ureas, 1-alkyl- and 1-alkenylazacyclo-alkanone derivatives (Lee et al., Critical Reviews in Therapeutic Drug Carrier Systems, 1991, page 92); and non-steroidal anti-inflammatory agents such as diclofenac sodium, indomethacin and phenylbutazone (Yamashita et al., J. Pharm. Pharmacol., 1987, 39, 621-626).

Carriers

Certain compositions of the present invention also incorporate carrier compounds in the formulation. As used herein, “carrier compound” or “carrier” can refer to a nucleic acid, or analog thereof, which is inert (i.e., does not possess biological activity per se) but is recognized as a nucleic acid by in vivo processes that reduce the bioavailability of a nucleic acid having biological activity by, for example, degrading the biologically active nucleic acid or promoting its removal from circulation. The co-administration of a nucleic acid and a carrier compound, typically with an excess of the latter substance, can result in a substantial reduction of the amount of nucleic acid recovered in the liver, kidney or other extracirculatory reservoirs, presumably due to competition between the carrier compound and the nucleic acid for a common receptor. For example, the recovery of a partially phosphorothioate dsRNA in hepatic tissue can be reduced when it is co-administered with polyinosinic acid, dextran sulfate, polycytidic acid or 4-acetamido-4′isothiocyano-stilbene-2,2′-disulfonic acid (Miyao et al., DsRNA Res. Dev., 1995, 5, 115-121; Takakura et al., DsRNA & Nucl. Acid Drug Dev., 1996, 6, 177-183.

Excipients

In contrast to a carrier compound, a “pharmaceutical carrier” or “excipient” is a pharmaceutically acceptable solvent, suspending agent or any other pharmacologically inert vehicle for delivering one or more nucleic acids to an animal. The excipient may be liquid or solid and is selected, with the planned manner of administration in mind, so as to provide for the desired bulk, consistency, etc., when combined with a nucleic acid and the other components of a given pharmaceutical composition. Typical pharmaceutical carriers include, but are not limited to, binding agents (e.g., pre-gelatinized maize starch, polyvinylpyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrants (e.g., starch, sodium starch glycolate, etc.); and wetting agents (e.g., sodium lauryl sulphate, etc).

Pharmaceutically acceptable organic or inorganic excipients suitable for non-parenteral administration which do not deleteriously react with nucleic acids can also be used to formulate the compositions of the present invention. Suitable pharmaceutically acceptable carriers include, but are not limited to, water, salt solutions, alcohols, polyethylene glycols, gelatin, lactose, amylose, magnesium stearate, talc, silicic acid, viscous paraffin, hydroxymethylcellulose, polyvinylpyrrolidone and the like.

Formulations for topical administration of nucleic acids may include sterile and non-sterile aqueous solutions, non-aqueous solutions in common solvents such as alcohols, or solutions of the nucleic acids in liquid or solid oil bases. The solutions may also contain buffers, diluents and other suitable additives. Pharmaceutically acceptable organic or inorganic excipients suitable for non-parenteral administration which do not deleteriously react with nucleic acids can be used.

Suitable pharmaceutically acceptable excipients include, but are not limited to, water, salt solutions, alcohol, polyethylene glycols, gelatin, lactose, amylose, magnesium stearate, talc, silicic acid, viscous paraffin, hydroxymethylcellulose, polyvinylpyrrolidone and the like.

Other Components

The compositions of the present invention may additionally contain other adjunct components conventionally found in pharmaceutical compositions, at their art-established usage levels. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Aqueous suspensions may contain substances which increase the viscosity of the suspension including, for example, sodium carboxymethylcellulose, sorbitol and/or dextran. The suspension may also contain stabilizers.

In some embodiments, pharmaceutical compositions featured in the invention include (a) one or more dsRNA compounds and (b) one or more anti-cytokine biologic agents which function by a non-RNAi mechanism. Examples of such biologics include, biologics that target IL1β (e.g., anakinra), IL6 (tocilizumab), or TNF (etanercept, infliximab, adlimumab, or certolizumab).

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit high therapeutic indices are preferred.

The data obtained from cell culture assays and animal studies can be used in formulating a range of dosage for use in humans. The dosage of compositions featured in the invention lies generally within a range of circulating concentrations that include the ED50 with little or no toxicity. The dosage may vary within this range depending upon the dosage form employed and the route of administration utilized. For any compound used in the methods featured in the invention, the therapeutically effective dose can be estimated initially from cell culture assays. A dose may be formulated in animal models to achieve a circulating plasma concentration range of the compound or, when appropriate, of the polypeptide product of a target sequence (e.g., achieving a decreased concentration of the polypeptide) that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

In addition to their administration, as discussed above, the dsRNAs featured in the invention can be administered in combination with other known agents effective in treatment of pathological processes mediated by APOC3 expression. In any event, the administering physician can adjust the amount and timing of dsRNA administration on the basis of results observed using standard measures of efficacy known in the art or described herein.

Methods for Inhibiting Expression of an APOC3 Gene

The present invention also provides methods of using a dsRNA of the invention and/or a composition containing an iRNA of the invention to reduce and/or inhibit APOC3 expression in a cell. The methods include contacting the cell with a dsRNA of the invention and maintaining the cell for a time sufficient to obtain degradation of the mRNA transcript of an APOC3 gene, thereby inhibiting expression of the APOC3 gene in the cell. Reduction in gene expression can be assessed by any methods known in the art. For example, a reduction in the expression of APOC3 may be determined by determining the mRNA expression level of APOC3 using methods routine to one of ordinary skill in the art, e.g., Northern blotting, qRT-PCR, by determining the protein level of APOC3 using methods routine to one of ordinary skill in the art, such as Western blotting, immunological techniques, and/or by determining a biological activity of APOC3, such as affecting one or more molecules associated with triglyceride levels, e.g., lipoproteinlipase (LPL) and/or hepatic lipase, or in an in vivo setting, a triglyceride level itself.

In the methods of the invention the cell may be contacted in vitro or in vivo, i.e., the cell may be within a subject.

A cell suitable for treatment using the methods of the invention may be any cell that expresses an APOC3 gene. A cell suitable for use in the methods of the invention may be a mammalian cell, e.g., a primate cell (such as a human cell or a non-human primate cell, e.g., a monkey cell or a chimpanzee cell), a non-primate cell (such as a cow cell, a pig cell, a camel cell, a llama cell, a horse cell, a goat cell, a rabbit cell, a sheep cell, a hamster, a guinea pig cell, a cat cell, a dog cell, a rat cell, a mouse cell, a lion cell, a tiger cell, a bear cell, or a buffalo cell), a bird cell (e.g., a duck cell or a goose cell), or a whale cell. In one embodiment, the cell is a human cell, e.g., a human liver cell.

APOC3 expression is inhibited in the cell by at least about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or about 100%.

The in vivo methods of the invention may include administering to a subject a composition containing a dsRNA, where the dsRNA includes a nucleotide sequence that is complementary to at least a part of an RNA transcript of the APOC3 gene of the mammal to be treated. When the organism to be treated is a mammal such as a human, the composition can be administered by any means known in the art including, but not limited to oral, intraperitoneal, or parenteral routes, including intracranial (e.g., intraventricular, intraparenchymal and intrathecal), intravenous, intramuscular, subcutaneous, transdermal, airway (aerosol), nasal, rectal, and topical (including buccal and sublingual) administration. In certain embodiments, the compositions are administered by intravenous infusion or injection or subcutaneous injection.

In some embodiments, the administration is via a depot injection. A depot injection may release the dsRNA in a consistent way over a prolonged time period. Thus, a depot injection may reduce the frequency of dosing needed to obtain a desired effect, e.g., a desired inhibition of APOC3, or a therapeutic or prophylactic effect. A depot injection may also provide more consistent serum concentrations. Depot injections may include subcutaneous injections or intramuscular injections. In preferred embodiments, the depot injection is a subcutaneous injection.

In some embodiments, the administration is via a pump. The pump may be an external pump or a surgically implanted pump. In certain embodiments, the pump is a subcutaneously implanted osmotic pump. In other embodiments, the pump is an infusion pump. An infusion pump may be used for intravenous, subcutaneous, arterial, or epidural infusions. In preferred embodiments, the infusion pump is a subcutaneous infusion pump. In other embodiments, the pump is a surgically implanted pump that delivers the dsRNA to the liver.

The mode of administration may be chosen based upon whether local or systemic treatment is desired and based upon the area to be treated. The route and site of administration may be chosen to enhance targeting.

In one aspect, the present invention also provides methods for inhibiting the expression of an APOC3 gene in a mammal. The methods include administering to the mammal a composition comprising a dsRNA that targets an APOC3 gene in a cell of the mammal and maintaining the mammal for a time sufficient to obtain degradation of the mRNA transcript of the APOC3 gene, thereby inhibiting expression of the APOC3 gene in the cell. Reduction in gene expression can be assessed by any methods known it the art and by methods, e.g. qRT-PCR, described herein. Reduction in protein production can be assessed by any methods known it the art and by methods, e.g. ELISA, described herein. In one embodiment, a puncture liver biopsy sample serves as the tissue material for monitoring the reduction in APOC3 gene and/or protein expression. In other embodiments, inhibition of the expression of an APOC3 gene is monitored indirectly by, for example, determining the expression and/or activity of a gene in an APOC3 pathway. For example, the activity of lipoprotein lipase (LPL) or hepatic lipase can be monitored to determine the inhibition of expression of an APOC3 gene. Triglyceride levels in a sample, e.g., a blood or liver sample, may also be measured. Inhibition of APOC3 inhibition can also be monitored by observing the effect on clinical presentations of elevated triglyceride levels, e.g., the effect on premature chronic heart disease (CHD), eruptive xanthoma, hepatosplenomegaly, and pancreatitis. Suitable assays are further described in the Examples section below.

The present invention further provides methods of treatment of a subject in need thereof. The treatment methods of the invention include administering a dsRNA of the invention to a subject, e.g., a subject that would benefit from a reduction and/or inhibition of APOC3 expression in a therapeutically effective amount of a dsRNA targeting an APOC3 gene or a pharmaceutical composition comprising a dsRNA targeting an APOC3 gene.

A dsRNA of the invention may be administered in “naked” form, or as a “free dsRNA.” A naked dsRNA is administered in the absence of a pharmaceutical composition. The naked dsRNA may be in a suitable buffer solution. The buffer solution may comprise acetate, citrate, prolamine, carbonate, or phosphate, or any combination thereof. In one embodiment, the buffer solution is phosphate buffered saline (PBS). The pH and osmolarity of the buffer solution containing the dsRNA can be adjusted such that it is suitable for administering to a subject. Additional buffers are described above.

Alternatively, a dsRNA of the invention may be administered as a pharmaceutical composition, such as a dsRNA liposomal formulation. Additional liposomal formulations are described herein.

Subjects that would benefit from a reduction and/or inhibition of APOC3 gene expression are those having elevated triglyceride levels, e.g., TG>150 mg/dL or those with severe hypertriglyceridemia, e.g., TG>500 mg/dL. In one embodiment, a subject has an APOC3 gene variant with a gain of function mutation. In other embodiments, the patient has mixed HTG (Type V) decreased LPL activity and/or Familial HTG (IV) inactivating LPL mutations and/or familial combined increased ApoB-100 levels. In another embodiments, the subject has uncontrolled hypertriglyceridemia with acute pancreatitis, or the subject is an HIV patient on therapy, or the subject has a high fat diet (postprandial hypertriglyceridemia), or a metabolic syndrome, or compound treatment (retinoid therapy), or insulin resistance. Treatment of a subject that would benefit from a reduction and/or inhibition of APOC3 gene expression includes therapeutic and prophylactic treatment.

The invention further provides methods for the use of a dsRNA or a pharmaceutical composition thereof, e.g., for treating a subject that would benefit from reduction and/or inhibition of APOC3 expression, e.g., a subject having elevated triglyceride levels, in combination with other pharmaceuticals and/or other therapeutic methods, e.g., with known pharmaceuticals and/or known therapeutic methods, such as, for example, those which are currently employed for treating elevated triglyceride levels. For example, in certain embodiments, a dsRNA targeting APOC3 is administered in combination with, e.g., an agent useful in treating elevated triglyceride levels. For example, additional therapeutics and therapeutic methods suitable for treating a subject that would benefit from reduction in APOC3 expression, e.g., a subject having elevated triglyceride levels, include lifestyle and diet modification, prescription grade fish oil, fibrates, niacin, ApoC3 antisense, CETP inhibitors, bile acid sequestrants, nicotinic acid, HMG CoA reductase inhibitors, Gemfibrozil, Fenofibrate, Cholesterol absorption inhibitors, neomycin, omega 3 fatty acids, and the like. The dsRNA and an additional therapeutic agent and/or treatment may be administered at the same time and/or in the same combination, e.g., parenterally, or the additional therapeutic agent can be administered as part of a separate composition or at separate times and/or by another method known in the art or described herein.

In one embodiment, the method includes administering a composition featured herein such that expression of the target APOC3 gene is decreased for about 1, 2, 3, 4, 5, 6, 7, 8, 12, 16, 18, or 24 hours or 28, 32, or 36 hours. In one embodiment, expression of the target APOC3 gene is decreased for an extended duration, e.g., at least about two, three, four days or more, e.g., about one week, two weeks, three weeks, or four weeks or longer.

Administration of the dsRNA according to the methods of the invention may result in a reduction of the severity, signs, symptoms, and/or markers of such diseases or disorders in a patient with elevated triglyceride levels. By “reduction” in this context is meant a statistically significant decrease in such level. The reduction can be, for example, at least about 5%, 10%, 15%, 20%, 25%, 30%, 35%, 40%, 45%, 50%, 55%, 60%, 65%, 70%, 75%, 80%, 85%, 90%, 95%, or about 100%.

Efficacy of treatment or prevention of disease can be assessed, for example by measuring disease progression, disease remission, symptom severity, quality of life, dose of a medication required to sustain a treatment effect, level of a disease marker or any other measurable parameter appropriate for a given disease being treated or targeted for prevention. It is well within the ability of one skilled in the art to monitor efficacy of treatment or prevention by measuring any one of such parameters, or any combination of parameters. For example, efficacy of treatment of elevated triglyceride levels may be assessed, for example, by periodic measurement of serum triglyceride levels. Comparisons of the later readings with the initial readings provide a physician an indication of whether the treatment is effective. It is well within the ability of one skilled in the art to monitor efficacy of treatment or prevention by measuring any one of such parameters, or any combination of parameters. In connection with the administration of a dsRNA targeting APOC3 or pharmaceutical composition thereof, “effective against” an elevated triglyceride levels indicates that administration in a clinically appropriate manner results in a beneficial effect for at least a statistically significant fraction of patients, such as a improvement of symptoms, a cure, a reduction in disease, extension of life, improvement in quality of life, or other effect generally recognized as positive by medical doctors familiar with elevated triglyceride levels and the related causes.

A treatment or preventive effect is evident when there is a statistically significant improvement in one or more parameters of disease status, or by a failure to worsen or to develop symptoms where they would otherwise be anticipated. As an example, a favorable change of at least 10% in a measurable parameter of disease, and preferably at least 20%, 30%, 40%, 50% or more can be indicative of effective treatment. Efficacy for a given dsRNA drug or formulation of that drug can also be judged using an experimental animal model for the given disease as known in the art. When using an experimental animal model, efficacy of treatment is evidenced when a statistically significant reduction in a marker or symptom is observed.

Alternatively, the efficacy can be measured by a reduction in the severity of disease as determined by one skilled in the art of diagnosis based on a clinically accepted disease severity grading scale, as but one example the Child-Pugh score (sometimes the Child-Turcotte-Pugh score). Any positive change resulting in e.g., lessening of severity of disease measured using the appropriate scale, represents adequate treatment using a dsRNA or dsRNA formulation as described herein.

Subjects can be administered a therapeutic amount of dsRNA, such as about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg, 0.1 mg/kg, 0.15 mg/kg, 0.2 mg/kg, 0.25 mg/kg, 0.3 mg/kg, 0.35 mg/kg, 0.4 mg/kg, 0.45 mg/kg, 0.5 mg/kg, 0.55 mg/kg, 0.6 mg/kg, 0.65 mg/kg, 0.7 mg/kg, 0.75 mg/kg, 0.8 mg/kg, 0.85 mg/kg, 0.9 mg/kg, 0.95 mg/kg, 1.0 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg, 2.1 mg/kg, 2.2 mg/kg, 2.3 mg/kg, 2.4 mg/kg, 2.5 mg/kg dsRNA, 2.6 mg/kg dsRNA, 2.7 mg/kg dsRNA, 2.8 mg/kg dsRNA, 2.9 mg/kg dsRNA, 3.0 mg/kg dsRNA, 3.1 mg/kg dsRNA, 3.2 mg/kg dsRNA, 3.3 mg/kg dsRNA, 3.4 mg/kg dsRNA, 3.5 mg/kg dsRNA, 3.6 mg/kg dsRNA, 3.7 mg/kg dsRNA, 3.8 mg/kg dsRNA, 3.9 mg/kg dsRNA, 4.0 mg/kg dsRNA, 4.1 mg/kg dsRNA, 4.2 mg/kg dsRNA, 4.3 mg/kg dsRNA, 4.4 mg/kg dsRNA, 4.5 mg/kg dsRNA, 4.6 mg/kg dsRNA, 4.7 mg/kg dsRNA, 4.8 mg/kg dsRNA, 4.9 mg/kg dsRNA, 5.0 mg/kg dsRNA, 5.1 mg/kg dsRNA, 5.2 mg/kg dsRNA, 5.3 mg/kg dsRNA, 5.4 mg/kg dsRNA, 5.5 mg/kg dsRNA, 5.6 mg/kg dsRNA, 5.7 mg/kg dsRNA, 5.8 mg/kg dsRNA, 5.9 mg/kg dsRNA, 6.0 mg/kg dsRNA, 6.1 mg/kg dsRNA, 6.2 mg/kg dsRNA, 6.3 mg/kg dsRNA, 6.4 mg/kg dsRNA, 6.5 mg/kg dsRNA, 6.6 mg/kg dsRNA, 6.7 mg/kg dsRNA, 6.8 mg/kg dsRNA, 6.9 mg/kg dsRNA, 7.0 mg/kg dsRNA, 7.1 mg/kg dsRNA, 7.2 mg/kg dsRNA, 7.3 mg/kg dsRNA, 7.4 mg/kg dsRNA, 7.5 mg/kg dsRNA, 7.6 mg/kg dsRNA, 7.7 mg/kg dsRNA, 7.8 mg/kg dsRNA, 7.9 mg/kg dsRNA, 8.0 mg/kg dsRNA, 8.1 mg/kg dsRNA, 8.2 mg/kg dsRNA, 8.3 mg/kg dsRNA, 8.4 mg/kg dsRNA, 8.5 mg/kg dsRNA, 8.6 mg/kg dsRNA, 8.7 mg/kg dsRNA, 8.8 mg/kg dsRNA, 8.9 mg/kg dsRNA, 9.0 mg/kg dsRNA, 9.1 mg/kg dsRNA, 9.2 mg/kg dsRNA, 9.3 mg/kg dsRNA, 9.4 mg/kg dsRNA, 9.5 mg/kg dsRNA, 9.6 mg/kg dsRNA, 9.7 mg/kg dsRNA, 9.8 mg/kg dsRNA, 9.9 mg/kg dsRNA, 9.0 mg/kg dsRNA, 10 mg/kg dsRNA, 15 mg/kg dsRNA, 20 mg/kg dsRNA, 25 mg/kg dsRNA, 30 mg/kg dsRNA, 35 mg/kg dsRNA, 40 mg/kg dsRNA, 45 mg/kg dsRNA, or about 50 mg/kg dsRNA. Values and ranges intermediate to the recited values are also intended to be part of this invention.

In certain embodiments, for example, when a composition of the invention comprises a dsRNA as described herein and a lipid, subjects can be administered a therapeutic amount of dsRNA, such as about 0.01 mg/kg to about 5 mg/kg, about 0.01 mg/kg to about 10 mg/kg, about 0.05 mg/kg to about 5 mg/kg, about 0.05 mg/kg to about 10 mg/kg, about 0.1 mg/kg to about 5 mg/kg, about 0.1 mg/kg to about 10 mg/kg, about 0.2 mg/kg to about 5 mg/kg, about 0.2 mg/kg to about 10 mg/kg, about 0.3 mg/kg to about 5 mg/kg, about 0.3 mg/kg to about 10 mg/kg, about 0.4 mg/kg to about 5 mg/kg, about 0.4 mg/kg to about 10 mg/kg, about 0.5 mg/kg to about 5 mg/kg, about 0.5 mg/kg to about 10 mg/kg, about 1 mg/kg to about 5 mg/kg, about 1 mg/kg to about 10 mg/kg, about 1.5 mg/kg to about 5 mg/kg, about 1.5 mg/kg to about 10 mg/kg, about 2 mg/kg to about 2.5 mg/kg, about 2 mg/kg to about 10 mg/kg, about 3 mg/kg to about 5 mg/kg, about 3 mg/kg to about 10 mg/kg, about 3.5 mg/kg to about 5 mg/kg, about 4 mg/kg to about 5 mg/kg, about 4.5 mg/kg to about 5 mg/kg, about 4 mg/kg to about 10 mg/kg, about 4.5 mg/kg to about 10 mg/kg, about 5 mg/kg to about 10 mg/kg, about 5.5 mg/kg to about 10 mg/kg, about 6 mg/kg to about 10 mg/kg, about 6.5 mg/kg to about 10 mg/kg, about 7 mg/kg to about 10 mg/kg, about 7.5 mg/kg to about 10 mg/kg, about 8 mg/kg to about 10 mg/kg, about 8.5 mg/kg to about 10 mg/kg, about 9 mg/kg to about 10 mg/kg, or about 9.5 mg/kg to about 10 mg/kg. Values and ranges intermediate to the recited values are also intended to be part of this invention.

For example, the dsRNA may be administered at a dose of about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, or about 10 mg/kg. Values and ranges intermediate to the recited values are also intended to be part of this invention.

In other embodiments, for example, when a composition of the invention comprises a dsRNA as described herein and an N-acetylgalactosamine, subjects can be administered a therapeutic amount of dsRNA, such as a dose of about 0.1 to about 50 mg/kg, about 0.25 to about 50 mg/kg, about 0.5 to about 50 mg/kg, about 0.75 to about 50 mg/kg, about 1 to about 50 mg/mg, about 1.5 to about 50 mg/kb, about 2 to about 50 mg/kg, about 2.5 to about 50 mg/kg, about 3 to about 50 mg/kg, about 3.5 to about 50 mg/kg, about 4 to about 50 mg/kg, about 4.5 to about 50 mg/kg, about 5 to about 50 mg/kg, about 7.5 to about 50 mg/kg, about 10 to about 50 mg/kg, about 15 to about 50 mg/kg, about 20 to about 50 mg/kg, about 20 to about 50 mg/kg, about 25 to about 50 mg/kg, about 25 to about 50 mg/kg, about 30 to about 50 mg/kg, about 35 to about 50 mg/kg, about 40 to about 50 mg/kg, about 45 to about 50 mg/kg, about 0.1 to about 45 mg/kg, about 0.25 to about 45 mg/kg, about 0.5 to about 45 mg/kg, about 0.75 to about 45 mg/kg, about 1 to about 45 mg/mg, about 1.5 to about 45 mg/kb, about 2 to about 45 mg/kg, about 2.5 to about 45 mg/kg, about 3 to about 45 mg/kg, about 3.5 to about 45 mg/kg, about 4 to about 45 mg/kg, about 4.5 to about 45 mg/kg, about 5 to about 45 mg/kg, about 7.5 to about 45 mg/kg, about 10 to about 45 mg/kg, about 15 to about 45 mg/kg, about 20 to about 45 mg/kg, about 20 to about 45 mg/kg, about 25 to about 45 mg/kg, about 25 to about 45 mg/kg, about 30 to about 45 mg/kg, about 35 to about 45 mg/kg, about 40 to about 45 mg/kg, about 0.1 to about 40 mg/kg, about 0.25 to about 40 mg/kg, about 0.5 to about 40 mg/kg, about 0.75 to about 40 mg/kg, about 1 to about 40 mg/mg, about 1.5 to about 40 mg/kb, about 2 to about 40 mg/kg, about 2.5 to about 40 mg/kg, about 3 to about 40 mg/kg, about 3.5 to about 40 mg/kg, about 4 to about 40 mg/kg, about 4.5 to about 40 mg/kg, about 5 to about 40 mg/kg, about 7.5 to about 40 mg/kg, about 10 to about 40 mg/kg, about 15 to about 40 mg/kg, about 20 to about 40 mg/kg, about 20 to about 40 mg/kg, about 25 to about 40 mg/kg, about 25 to about 40 mg/kg, about 30 to about 40 mg/kg, about 35 to about 40 mg/kg, about 0.1 to about 30 mg/kg, about 0.25 to about 30 mg/kg, about 0.5 to about 30 mg/kg, about 0.75 to about 30 mg/kg, about 1 to about 30 mg/mg, about 1.5 to about 30 mg/kb, about 2 to about 30 mg/kg, about 2.5 to about 30 mg/kg, about 3 to about 30 mg/kg, about 3.5 to about 30 mg/kg, about 4 to about 30 mg/kg, about 4.5 to about 30 mg/kg, about 5 to about 30 mg/kg, about 7.5 to about 30 mg/kg, about 10 to about 30 mg/kg, about 15 to about 30 mg/kg, about 20 to about 30 mg/kg, about 20 to about 30 mg/kg, about 25 to about 30 mg/kg, about 0.1 to about 20 mg/kg, about 0.25 to about 20 mg/kg, about 0.5 to about 20 mg/kg, about 0.75 to about 20 mg/kg, about 1 to about 20 mg/mg, about 1.5 to about 20 mg/kb, about 2 to about 20 mg/kg, about 2.5 to about 20 mg/kg, about 3 to about 20 mg/kg, about 3.5 to about 20 mg/kg, about 4 to about 20 mg/kg, about 4.5 to about 20 mg/kg, about 5 to about 20 mg/kg, about 7.5 to about 20 mg/kg, about 10 to about 20 mg/kg, or about 15 to about 20 mg/kg. Values and ranges intermediate to the recited values are also intended to be part of this invention.

For example, subjects can be administered a therapeutic amount of dsRNA, such as about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10.5, 11, 11.5, 12, 12.5, 13, 13.5, 14, 14.5, 15, 15.5, 16, 16.5, 17, 17.5, 18, 18.5, 19, 19.5, 20, 21, 22, 23, 24, 25, 26, 27, 28, 29, 30, 31, 32, 33, 34, 34, 35, 36, 37, 38, 39, 40, 41, 42, 43, 44, 45, 46, 47, 48, 49, or about 50 mg/kg. Values and ranges intermediate to the recited values are also intended to be part of this invention.

The dsRNA can be administered by intravenous infusion over a period of time, such as over a 5, 6, 7, 8, 9, 10, 11, 12, 13, 14, 15, 16, 17, 18, 19, 20, 21, 22, 23, 24, or about a 25 minute period. The administration may be repeated, for example, on a regular basis, such as biweekly (i.e., every two weeks) for one month, two months, three months, four months or longer. After an initial treatment regimen, the treatments can be administered on a less frequent basis. For example, after administration biweekly for three months, administration can be repeated once per month, for six months or a year or longer. Administration of the dsRNA can reduce APOC3 levels, e.g., in a cell, tissue, blood, urine or other compartment of the patient by at least about 5%, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 39, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or at least about 99% or more.

Before administration of a full dose of the dsRNA, patients can be administered a smaller dose, such as a 5% infusion reaction, and monitored for adverse effects, such as an allergic reaction. In another example, the patient can be monitored for unwanted immunostimulatory effects, such as increased cytokine (e.g., TNF-alpha or INF-alpha) levels.

Owing to the inhibitory effects on APOC3 expression, a composition according to the invention or a pharmaceutical composition prepared there from can enhance the quality of life.

Unless otherwise defined, all technical and scientific terms used herein have the same meaning as commonly understood by one of ordinary skill in the art to which this invention belongs. Although methods and materials similar or equivalent to those described herein can be used in the practice or testing of the dsRNAs and methods featured in the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### EXAMPLES

### Example 1. dsRNA Synthesis

Source of Reagents

Where the source of a reagent is not specifically given herein, such reagent may be obtained from any supplier of reagents for molecular biology at a quality/purity standard for application in molecular biology.

siRNA Synthesis

Single-stranded RNAs were produced by solid phase synthesis on a scale of 1 μmole using an Expedite 8909 synthesizer (Applied Biosystems, Applera Deutschland GmbH, Darmstadt, Germany) and controlled pore glass (CPG, 500 Å, Proligo Biochemie GmbH, Hamburg, Germany) as solid support. RNA and RNA containing 2′-O-methyl nucleotides were generated by solid phase synthesis employing the corresponding phosphoramidites and 2′-O-methyl phosphoramidites, respectively (Proligo Biochemie GmbH, Hamburg, Germany). These building blocks were incorporated at selected sites within the sequence of the oligoribonucleotide chain using standard nucleoside phosphoramidite chemistry such as described in Current protocols in nucleic acid chemistry, Beaucage, S. L. et al. (Edrs.), John Wiley & Sons, Inc., New York, NY, USA. Phosphorothioate linkages were introduced by replacement of the iodine oxidizer solution with a solution of the Beaucage reagent (Chruachem Ltd, Glasgow, UK) in acetonitrile (1%). Further ancillary reagents were obtained from Mallinckrodt Baker (Griesheim, Germany).

Deprotection and purification of the crude oligoribonucleotides by anion exchange HPLC were carried out according to established procedures. Yields and concentrations were determined by UV absorption of a solution of the respective RNA at a wavelength of 260 nm using a spectral photometer (DU 640B, Beckman Coulter GmbH, Unterschleißheim, Germany). Double stranded RNA was generated by mixing an equimolar solution of complementary strands in annealing buffer (20 mM sodium phosphate, pH 6.8; 100 mM sodium chloride), heated in a water bath at 85-90° C. for 3 minutes and cooled to room temperature over a period of 3-4 hours. The annealed RNA solution was stored at −20° C. until use.

Nucleic acid sequences are represented below using standard nomenclature, and specifically the abbreviations of Table B.

TABLE B
Abbreviations.

| Abbreviation | Nucleotide(s) |
|---|---|
| A | adenosine-3′-phosphate |
| C | cytidine-3′-phosphate |
| G | guanosine-3′-phosphate |
| U | uridine-3′-phosphate |
| N | any nucleotide (G, A, C, or T) |
| a | 2′-O-methyladenosine-3′-phosphate |
| c | 2′-O-methylcytidine-3′-phosphate |
| g | 2′-O-methylguanosine-3′-phosphate |
| u | 2′-O-methyluridine-3′-phosphate |
| T, dT | 2′-deoxythymidine-3′-phosphate |
| sT; sdT | 2′-deoxy-thymidine-5′phosphate-phosphorothioate |

### Example 2: APOC3 siRNA Design

Transcripts

siRNA design was carried out to identify siRNAs targeting all human and cynomolgus monkey (Macaca fascicularis; henceforth “cyno”) APOC3 transcripts annotated in the NCBI Gene database (http://www.ncbi.nlm.nih.gov/gene/). Design used the following transcripts from NCBI: Human-NM_000040.1; cyno-X68359.1. All siRNA duplexes were designed that shared 100% identity with the listed human and cyno transcripts.

siRNA Design, Specificity, and Efficacy Prediction

The siRNAs were selected based on predicted specificity, predicted efficacy, and GC content.

The predicted specificity of all possible 19mers was predicted from each sequence. Candidate 19mers were then selected that lacked repeats longer than 7 nucleotides. These 171 candidate siRNAs were used in a comprehensive search against the human transcriptome (defined as the set of NM_ and XM_records within the human NCBI Refseq set)

A score was calculated based on the position and number of mismatches between the siRNA and any potential ‘off-target’ transcript and comparing the frequency of heptamers and octomers derived from 3 distinct, seed (positions 2-9 from the 5′ end of the molecule)-derived hexamers of each oligo. Both siRNAs strands were assigned to a category of specificity according to the calculated scores: a score above 3 qualifies as highly specific, equal to 3 as specific and between 2.2 and 2.8 as moderately specific. We sorted by the specificity of the antisense strand. We then selected duplexes whose antisense oligos had less than 70% overall GC content, lacked GC at the first position, and did not match the mouse APOC3 transcript NM_023114.3.

siRNA Sequence Selection

A total of 27 sense and 27 antisense derived siRNA oligos were synthesized and formed into duplexes.

### Example 3. APOC3 siRNA Synthesis

Synthesis of Modified and Unmodified ApoC3 Sequences

APOC3 tiled sequences were synthesized on MerMade 192 synthesizer at either 1 or 0.2 umol scale.

Single strands and duplexes were made with either unmodified, 2′-O-Methyl or 2′-fluoro chemical modifications. Synthesis conditions were appropriately modified based on the nature of chemical modifications in the single strands.

Synthesis, Cleavage and Deprotection:

The synthesis of APOC3 sequences (unmodified, 2-O-Methyl or 2′-fluoro) used solid supported oligonucleotide synthesis using phosphoramidite chemistry.

The synthesis of the above sequences was performed at either 1 or 0.2 um scale in 96 well plates. Unmodified and modified (2-O-Methyl or 2′-fluoro) amidite solutions were prepared at 0.1M concentration and ethyl thio tetrazole (0.6M in Acetonitrile) was used as activator.

The synthesized sequences were cleaved and deprotected in 96 well plates, using either aqueous ammonia or aqueous methylamine in the first step and fluoride reagent in the second step. The crude sequences were precipitated using acetone:ethanol (80:20) mix and the pellet were re-suspended in 0.2M sodium acetate buffer to convert the crude single strands to their sodium salts. Samples from each sequence were analyzed by LC-MS to confirm the identity, UV for quantification and by IEX chromatography to determine purity.

Purification and Desalting:

APOC3 tiled sequences were precipitated and purified on AKTA Purifier system using Sephadex column. The process was run at ambient temperature. Sample injection and collection was performed in 96 well (1.8 mL-deep well) plates. A single peak corresponding to the full length sequence was collected in the eluent. The desalted APOC3 sequences were analyzed for concentration (by UV measurement at A260) and purity (by ion exchange HPLC). The complementary single strands were then combined in a 1:1 stoichiometric ratio to form siRNA duplexes.

Tables 1 and 2 provide a first set of unmodified and modified sequences.

### Example 4. APOC3 siRNA In Vitro Screening

Cell Culture and Transfections:

Hep3B cells (ATCC, Manassas, VA) were grown to near confluence at 37° C. in an atmosphere of 5% CO₂ in RPMI (ATCC) supplemented with 10% FBS, streptomycin, and glutamine (ATCC) before being released from the plate by trypsinization. Transfection was carried out by adding 14.8 μl of Opti-MEM plus 0.2 μL of Lipofectamine RNAiMax per well (Invitrogen, Carlsbad CA. cat #13778-150) to 5 μl of siRNA duplexes per well into a 96-well plate and incubated at room temperature for 15 minutes. 80 μl of complete growth media without antibiotic containing ˜2×10⁴ Hep3B cells were then added to the siRNA mixture. Cells were incubated for either 24 or 120 hours prior to RNA purification. Single dose experiments were performed at 10 nM and 0.1 nM final duplex concentration and dose response experiments were done at 10, 1, 0.5, 0.1, 0.05, 0.01, 0.005, 0.001, 0.0005, 0.0001, 0.00005, 0.00001 nM final duplex concentration.

Total RNA Isolation Using DYNABEADS mRNA Isolation Kit (Invitrogen, Part #: 610-12):

Cells were harvested and lysed in 150 μl of Lysis/Binding Buffer then mixed for 5 minute at 850 rpm using an Eppendorf Thermomixer (the mixing speed was the same throughout the process). Ten microliters of magnetic beads and 80 μl Lysis/Binding Buffer mixture were added to a round bottom plate and mixed for 1 minute. Magnetic beads were captured using magnetic stand and the supernatant was removed without disturbing the beads. After removing supernatant, the lysed cells were added to the remaining beads and mixed for 5 minutes. After removing supernatant, magnetic beads were washed 2 times with 150 μl Wash Buffer A and mixed for 1 minute. Beads were capture again and supernatant removed. Beads were then washed with 150 μl Wash Buffer B, captured and supernatant was removed. Beads were next washed with 150 μl Elution Buffer, captured and supernatant removed. Beads were allowed to dry for 2 minutes. After drying, 50 μl of Elution Buffer was added and mixed for 5 minutes at 70° C. Beads were captured on magnet for 5 minutes. 40 μl of supernatant was removed and added to another 96 well plate.

cDNA Synthesis Using ABI High Capacity cDNA Reverse Transcription Kit (Applied Biosystems, Foster City, CA, Cat #4368813):

A master mix of 2 μl 10× Buffer, 0.8 μl 25× dNTPs, 2 μl Random primers, 1 μl Reverse Transcriptase, 1 μl RNase inhibitor and 3.2 μl of H2O per reaction were added into 10 μl total RNA. cDNA was generated using a Bio-Rad C-1000 or S-1000 thermal cycler (Hercules, CA) through the following steps: 25° C. 10 min, 37° C. 120 min, 85° C. 5 sec, 4° C. hold.

Real Time PCR:

2 μl of cDNA were added to a master mix containing 0.5 μl GAPDH TaqMan Probe (Applied Biosystems Cat #4326317E), 0.5 μl ApoC3 TaqMan probe (Applied Biosystems cat #Hs00163644_ml) and 5 μl Lightcycler 480 probe master mix (Roche Cat #04887301001) per well in a 384 well 50 plates (Roche cat #04887301001). Real time PCR was done in an ABI 7900HT Real Time PCR system (Applied Biosystems) using the ΔΔCt(RQ) assay. Each duplex was tested in two independent transfections and each transfection was assayed in duplicate, unless otherwise noted in the summary tables.

To calculate relative fold change, real time data were analyzed using the ΔΔCt method and normalized to assays performed with cells transfected with 10 nM AD-1955, or mock transfected cells. IC50s were calculated using a 4 parameter fit model using XLFit and normalized to cells transfected with AD-1955 or naïve cells over the same dose range, or to its own lowest dose.

Viability Screens

Cell Viability was Measured on Days 3, 5 in HeLa and Hep3B Cells Following transfection with 100, 10, 1, 0.1, 0.01 and 0.0001 nM siRNA. Cells were plated at a density of 10,000 cells per well in 96 well plates. Each siRNA was assayed in triplicate and the data averaged. siRNAs targeting PLK1 and AD-19200 were included as positive controls for loss of viability and AD-1955 as a negative control. PLK1 and AD-19200 result in a dose dependant loss of viability. To measure viability, 20 ul of CellTiter Blue (Promega) was added to each well of the 96 well plates after 3, 5, days and incubated at 37° C. for 2 hours. Plates were then read in a Spectrophotomoeter (Molecular Devices) at 560Ex/590Em. Viability was expressed as the average value of light units from three replicate transfections+/−standard deviation. In some cases, relative viability was assessed by first averaging the three replicate transfections and then normalizing to the values obtained from the lowest dose (0.001 nM).

The results are provided in Tables 3, 4, and 5.

### Example 5: APOC3 In Vivo Testing in Mice

An siRNA targeting APOC3 was administered to mice, both wild type (5.0 mg/kg) and a transgenic hyperlipidemic model SREBPtg/LDLR−/− KO mice (1.0 mg/kg). Mice were sacrificed two days after administration and hepatic target mRNA, serum triglycerides, and serum total cholesterol levels were determined. An MC3 containing LNp11 formulation was used.

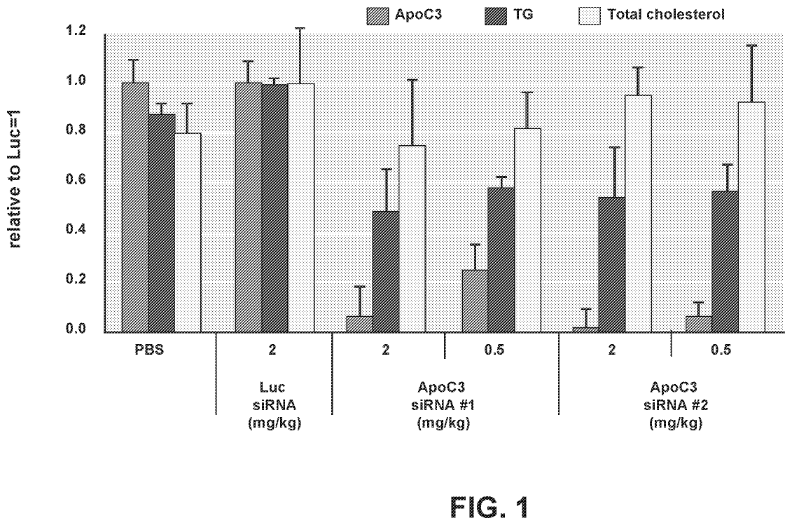

The results for wild-type mice are shown in FIG. 1. Administration of the siRNA targeting APOC3 resulted in a knock down in mRNA levels, a 50% lowering of triglycerides, and a lowering of total cholesterol in wild type mice. Administration of the siRNA targeting APOC3 resulted in a 80% lowering of triglycerides in the hyperlipidemic model mice, data not shown. The results demonstrate that APOC3 is a validated target for siRNA based treatment of hypertriglyceridemia, including coronary heart disease (CAD) and pancreatitis.

### Example 6: Synthesis and Screening of Modified siRNA Targeting APOC3 (Second Set

Additional modified APOC3 siRNA were synthesized as described in Table 6 and Table 7 using the methods described above. A UMdTdsdT modification pattern is a dT-phosphorothioate-dT addition to each strand. A DECAF modification pattern is as follows: Sense strand—2′O-methyls on all pyrimidines, dTsdT/dTdT overhang; Antisense strand—modify ‘U’ at any two sites in the dinucleotide motif UU/UA/UG in the seed region (positions 2-9) plus 2′O-Methyls on last 3 nucleotides (positions 17-19) plus 2′O-Methyls on every ‘U’ in positions 10-16; dTsdT/dTdT overhang. A FOME modification pattern is as follows: Sense strand—2′F (5′ first base) then alternating with 2′OMe, Antisense strand—2′OMe (5′ first base), then alternating with 2′F.

The siRNA described in Table 6 and Table 7 were assayed in Hep3b cells as described above. The results are shown in Table 8.

### Example 7: Synthesis and Screening of Modified siRNA Targeting APOC3 (Third Set

Additional modified APOC3 siRNA were synthesized as described in Table 9 and Table 10 using the methods described above. The siRNA were assayed in Hep3b cells as described above. The results are shown in Table 11.

TABLE 1
ApoC3 siRNA (first set): unmodified sequences

| | | SEQ | | SEQ | |
|---|---|---|---|---|---|
| | Position in | ID | | ID | |
| Duplex name | NM_000040.1 | NO: | Sense Sequence | NO: | Antisense Sequence |
| AD-24548.1UM | 264-282 | 2 | ACUGGAGCACCGUUAAGGA | 83 | UCCUUAACGGUGCUCCAGU |
| AD-24549.1UM | 417-435 | 3 | GCCCCUGUAGGUUGCUUAA | 84 | UUAAGCAACCUACAGGGGC |
| AD-24550.1UM | 418-436 | 4 | CCCCUGUAGGUUGCUUAAA | 85 | UUUAAGCAACCUACAGGGG |
| AD-24551.1UM | 47-65 | 5 | AUGCAGCCCCGGGUACUCC | 86 | GGAGUACCCGGGGCUGCAU |
| AD-24552.1UM | 412-430 | 6 | GGGCUGCCCCUGUAGGUUG | 87 | CAACCUACAGGGGCAGCCC |
| AD-24553.1UM | 267-285 | 7 | GGAGCACCGUUAAGGACAA | 88 | UUGUCCUUAACGGUGCUCC |
| AD-24554.1UM | 266-284 | 8 | UGGAGCACCGUUAAGGACA | 89 | UGUCCUUAACGGUGCUCCA |
| AD-24555.1UM | 423-441 | 9 | GUAGGUUGCUUAAAAGGGA | 90 | UCCCUUUUAAGCAACCUAC |
| AD-24556.1UM | 265-283 | 10 | CUGGAGCACCGUUAAGGAC | 91 | GUCCUUAACGGUGCUCCAG |
| AD-24557.1UM | 45-63 | 11 | CCAUGCAGCCCCGGGUACU | 92 | AGUACCCGGGGCUGCAUGG |
| AD-24558.1UM | 416-434 | 12 | UGCCCCUGUAGGUUGCUUA | 93 | UAAGCAACCUACAGGGGCA |
| AD-24559.1UM | 44-62 | 13 | GCCAUGCAGCCCCGGGUAC | 94 | GUACCCGGGGCUGCAUGGC |
| AD-24560.1UM | 263-281 | 14 | UACUGGAGCACCGUUAAGG | 95 | CCUUAACGGUGCUCCAGUA |
| AD-24561.1UM | 262-280 | 15 | CUACUGGAGCACCGUUAAG | 96 | CUUAACGGUGCUCCAGUAG |
| AD-24562.1UM | 261-279 | 16 | ACUACUGGAGCACCGUUAA | 97 | UUAACGGUGCUCCAGUAGU |
| AD-24563.1UM | 260-278 | 17 | GACUACUGGAGCACCGUUA | 98 | UAACGGUGCUCCAGUAGUC |
| AD-24564.1UM | 341-359 | 18 | GCCUGAGACCUCAAUACCC | 99 | GGGUAUUGAGGUCUCAGGC |
| AD-24565.1UM | 340-358 | 19 | UGCCUGAGACCUCAAUACC | 100 | GGUAUUGAGGUCUCAGGCA |
| AD-24566.1UM | 46-64 | 20 | CAUGCAGCCCCGGGUACUC | 101 | GAGUACCCGGGGCUGCAUG |
| AD-24567.1UM | 342-360 | 21 | CCUGAGACCUCAAUACCCC | 102 | GGGGUAUUGAGGUCUCAGG |
| AD-24568.1UM | 345-363 | 22 | GAGACCUCAAUACCCCAAG | 103 | CUUGGGGUAUUGAGGUCUC |
| AD-24569.1UM | 249-267 | 23 | GUUCCCUGAAAGACUACUG | 104 | CAGUAGUCUUUCAGGGAAC |
| AD-24570.1UM | 411-429 | 24 | AGGGCUGCCCCUGUAGGUU | 105 | AACCUACAGGGGCAGCCCU |
| AD-24571.1UM | 339-357 | 25 | CUGCCUGAGACCUCAAUAC | 106 | GUAUUGAGGUCUCAGGCAG |
| AD-24572.1UM | 351-369 | 26 | UCAAUACCCCAAGUCCACC | 107 | GGUGGACUUGGGGUAUUGA |
| AD-24573.1UM | 235-253 | 27 | GACCGAUGGCUUCAGUUCC | 108 | GGAACUGAAGCCAUCGGUC |
| AD-24574.1UM | 248-266 | 28 | AGUUCCCUGAAAGACUACU | 109 | AGUAGUCUUUCAGGGAACU |
| AD-24575.1UM | 415-433 | 29 | CUGCCCCUGUAGGUUGCUU | 110 | AAGCAACCUACAGGGGCAG |
| AD-24576.1UM | 234-252 | 30 | UGACCGAUGGCUUCAGUUC | 111 | GAACUGAAGCCAUCGGUCA |
| AD-24577.1UM | 168-186 | 31 | AGACCGCCAAGGAUGCACU | 112 | AGUGCAUCCUUGGCGGUCU |
| AD-45078.1UM | 232-250 | 32 | GGUGACCGAUGGCUUCAGU | 113 | ACUGAAGCCAUCGGUCACCTT |
| AD-45084.1UM | 237-255 | 33 | CCGAUGGCUUCAGUUCCCU | 114 | AGGGAACUGAAGCCAUCGGTT |
| AD-45090.1UM | 239-257 | 34 | GAUGGCUUCAGUUCCCUGA | 115 | UCAGGGAACUGAAGCCAUCTT |
| AD-45096.1UM | 240-258 | 35 | AUGGCUUCAGUUCCCUGAA | 116 | UUCAGGGAACUGAAGCCAUTT |
| AD-45101.1UM | 48-66 | 36 | UGCAGCCCCGGGUACUCCU | 117 | AGGAGUACCCGGGGCUGCATT |
| AD-45102.1UM | 241-259 | 37 | UGGCUUCAGUUCCCUGAAA | 118 | UUUCAGGGAACUGAAGCCATT |
| AD-45107.1UM | 49-67 | 38 | GCAGCCCCGGGUACUCCUU | 119 | AAGGAGUACCCGGGGCUGCTT |
| AD-45108.1UM | 243-261 | 39 | GCUUCAGUUCCCUGAAAGA | 120 | UCUUUCAGGGAACUGAAGCTT |
| AD-45113.1UM | 166-184 | 40 | CAAGACCGCCAAGGAUGCA | 121 | UGCAUCCUUGGCGGUCUUGTT |
| AD-45114.1UM | 251-269 | 41 | UCCCUGAAAGACUACUGGA | 122 | UCCAGUAGUCUUUCAGGGATT |
| AD-45119.1UM | 230-248 | 42 | UGGGUGACCGAUGGCUUCA | 123 | UGAAGCCAUCGGUCACCCATT |
| AD-45120.1UM | 254-272 | 43 | CUGAAAGACUACUGGAGCA | 124 | UGCUCCAGUAGUCUUUCAGTT |
| AD-45121.1UM | 259-277 | 44 | AGACUACUGGAGCACCGUU | 125 | AACGGUGCUCCAGUAGUCU |
| AD-45122.1UM | 410-428 | 45 | CAGGGCUGCCCCUGUAGGU | 126 | ACCUACAGGGGCAGCCCUG |
| AD-45123.1UM | 49-67 | 46 | GCAGCCCCGGGUACUCCUU | 127 | AAGGAGUACCCGGGGCUGC |
| AD-45124.1UM | 243-261 | 47 | GCUUCAGUUCCCUGAAAGA | 128 | UCUUUCAGGGAACUGAAGC |
| AD-45125.1UM | 343-361 | 48 | CUGAGACCUCAAUACCCCA | 129 | UGGGGUAUUGAGGUCUCAG |
| AD-45126.1UM | 430-448 | 49 | GCUUAAAAGGGACAGUAUU | 130 | AAUACUGUCCCUUUUAAGC |
| AD-45127.1UM | 269-287 | 50 | AGCACCGUUAAGGACAAGU | 131 | ACUUGUCCUUAACGGUGCU |
| AD-45128.1UM | 414-432 | 51 | GCUGCCCCUGUAGGUUGCU | 132 | AGCAACCUACAGGGGCAGC |
| AD-45129.1UM | 166-184 | 52 | CAAGACCGCCAAGGAUGCA | 133 | UGCAUCCUUGGCGGUCUUG |
| AD-45130.1UM | 251-269 | 53 | UCCCUGAAAGACUACUGGA | 134 | UCCAGUAGUCUUUCAGGGA |
| AD-45131.1UM | 344-362 | 54 | UGAGACCUCAAUACCCCAA | 135 | UUGGGGUAUUGAGGUCUCA |
| AD-45132.1UM | 514-532 | 55 | CUGGACAAGAAGCUGCUAU | 136 | AUAGCAGCUUCUUGUCCAG |
| AD-45133.1UM | 270-288 | 56 | GCACCGUUAAGGACAAGUU | 137 | AACUUGUCCUUAACGGUGC |
| AD-45135.1UM | 230-248 | 57 | UGGGUGACCGAUGGCUUCA | 138 | UGAAGCCAUCGGUCACCCA |
| AD-45136.1UM | 254-272 | 58 | CUGAAAGACUACUGGAGCA | 139 | UGCUCCAGUAGUCUUUCAG |
| AD-45137.1UM | 349-367 | 59 | CCUCAAUACCCCAAGUCCA | 140 | UGGACUUGGGGUAUUGAGG |
| AD-45138.1UM | 337-355 | 60 | GGCUGCCUGAGACCUCAAU | 141 | AUUGAGGUCUCAGGCAGCC |
| AD-45139.1UM | 425-443 | 61 | AGGUUGCUUAAAAGGGACA | 142 | UGUCCCUUUUAAGCAACCU |
| AD-45140.1UM | 232-250 | 62 | GGUGACCGAUGGCUUCAGU | 143 | ACUGAAGCCAUCGGUCACC |
| AD-45141.1UM | 259-277 | 63 | AGACUACUGGAGCACCGUU | 144 | AACGGUGCUCCAGUAGUCU |
| AD-45143.1UM | 338-356 | 64 | GCUGCCUGAGACCUCAAUA | 145 | UAUUGAGGUCUCAGGCAGC |
| AD-45144.1UM | 429-447 | 65 | UGCUUAAAAGGGACAGUAU | 146 | AUACUGUCCCUUUUAAGCA |
| AD-45145.1UM | 237-255 | 66 | CCGAUGGCUUCAGUUCCCU | 147 | AGGGAACUGAAGCCAUCGG |
| AD-45146.1UM | 269-287 | 67 | AGCACCGUUAAGGACAAGU | 148 | ACUUGUCCUUAACGGUGCU |
| AD-45147.1UM | 414-432 | 68 | GCUGCCCCUGUAGGUUGCU | 149 | AGCAACCUACAGGGGCAGC |
| AD-45148.1UM | 343-361 | 69 | CUGAGACCUCAAUACCCCA | 150 | UGGGGUAUUGAGGUCUCAG |
| AD-45149.1UM | 430-448 | 70 | GCUUAAAAGGGACAGUAUU | 151 | AAUACUGUCCCUUUUAAGC |
| AD-45150.1UM | 239-257 | 71 | GAUGGCUUCAGUUCCCUGA | 152 | UCAGGGAACUGAAGCCAUC |
| AD-45151.1UM | 270-288 | 72 | GCACCGUUAAGGACAAGUU | 153 | AACUUGUCCUUAACGGUGC |
| AD-45152.1UM | 419-437 | 73 | CCCUGUAGGUUGCUUAAAA | 154 | UUUUAAGCAACCUACAGGG |
| AD-45153.1UM | 344-362 | 74 | UGAGACCUCAAUACCCCAA | 155 | UUGGGGUAUUGAGGUCUCA |
| AD-45154.1UM | 514-532 | 75 | CUGGACAAGAAGCUGCUAU | 156 | AUAGCAGCUUCUUGUCCAG |
| AD-45155.1UM | 240-258 | 76 | AUGGCUUCAGUUCCCUGAA | 157 | UUCAGGGAACUGAAGCCAU |
| AD-45157.1UM | 425-443 | 77 | AGGUUGCUUAAAAGGGACA | 158 | UGUCCCUUUUAAGCAACCU |
| AD-45158.1UM | 349-367 | 78 | CCUCAAUACCCCAAGUCCA | 159 | UGGACUUGGGGUAUUGAGG |
| AD-45159.1UM | 48-66 | 79 | UGCAGCCCCGGGUACUCCU | 160 | AGGAGUACCCGGGGCUGCA |
| AD-45160.1UM | 241-259 | 80 | UGGCUUCAGUUCCCUGAAA | 161 | UUUCAGGGAACUGAAGCCA |
| AD-45161.1UM | 338-356 | 81 | GCUGCCUGAGACCUCAAUA | 162 | UAUUGAGGUCUCAGGCAGC |
| AD-45162.1UM | 429-447 | 82 | UGCUUAAAAGGGACAGUAU | 163 | AUACUGUCCCUUUUAAGCA |

TABLE 2
ApoC3 modified siRNA (first set) sequences
Lowercase nucleotides (g, a, u, c) are 2′-O-methyl nucleotides;
Nf (e.g., Gf, Af, Uf, Cf)
is a 2′-fluoro nucleotide; s is a phosphothiorate linkage.

| | SEQ | | SEQ | |
|---|---|---|---|---|
| Duplex | ID | | ID | |
| name | NO: | Sense Sequence | NO: | Antisense Sequence |
| AD-24548.1 | 164 | AcuGGAGcAccGuuAAGGAdTsdT | 245 | UCCUuAACGGUGCUCcAGUdTsdT |
| AD-24549.1 | 165 | GccccuGuAGGuuGcuuAAdTsdT | 246 | UuAAGcAACCuAcAGGGGCdTsdT |
| AD-24550.1 | 166 | ccccuGuAGGuuGcuuAAAdTsdT | 247 | UUuAAGcAACCuAcAGGGGdTsdT |
| AD-24551.1 | 167 | AuGcAGccccGGGuAcuccdTsdT | 248 | GGAGuACCCGGGGCUGcAUdTsdT |
| AD-24552.1 | 168 | GGGcuGccccuGuAGGuuGdTsdT | 249 | cAACCuAcAGGGGcAGCCCdTsdT |
| AD-24553.1 | 169 | GGAGcAccGuuAAGGAcAAdTsdT | 250 | UUGUCCUuAACGGUGCUCCdTsdT |
| AD-24554.1 | 170 | uGGAGcAccGuuAAGGAcAdTsdT | 251 | UGUCCUuAACGGUGCUCcAdTsdT |
| AD-24556.1 | 171 | cuGGAGcAccGuuAAGGAcdTsdT | 252 | GUCCUuAACGGUGCUCcAGdTsdT |
| AD-24557.1 | 172 | ccAuGcAGccccGGGuAcudTsdT | 253 | AGuACCCGGGGCUGcAUGGdTsdT |
| AD-24558.1 | 173 | uGccccuGuAGGuuGcuuAdTsdT | 254 | uAAGcAACCuAcAGGGGcAdTsdT |
| AD-24559.1 | 174 | GccAuGcAGccccGGGuAcdTsdT | 255 | GuACCCGGGGCUGcAUGGCdTsdT |
| AD-24560.1 | 175 | uAcuGGAGcAccGuuAAGGdTsdT | 256 | CCUuAACGGUGCUCcAGuAdTsdT |
| AD-24561.1 | 176 | cuAcuGGAGcAccGuuAAGdTsdT | 257 | CUuAACGGUGCUCcAGuAGdTsdT |
| AD-24563.1 | 177 | GAcuAcuGGAGcAccGuuAdTsdT | 258 | uAACGGUGCUCcAGuAGUCdTsdT |
| AD-24564.1 | 178 | GccuGAGAccucAAuAcccdTsdT | 259 | GGGuAUUGAGGUCUcAGGCdTsdT |
| AD-24565.1 | 179 | uGccuGAGAccucAAuAccdTsdT | 260 | GGuAUUGAGGUCUcAGGcAdTsdT |
| AD-24566.1 | 180 | cAuGcAGccccGGGuAcucdTsdT | 261 | GAGuACCCGGGGCUGcAUGdTsdT |
| AD-24567.1 | 181 | ccuGAGAccucAAuAccccdTsdT | 262 | GGGGuAUUGAGGUCUcAGGdTsdT |
| AD-24568.1 | 182 | GAGAccucAAuAccccAAGdTsdT | 263 | CUUGGGGuAUUGAGGUCUCdTsdT |
| AD-24569.1 | 183 | GuucccuGAAAGAcuAcuGdTsdT | 264 | cAGuAGUCUUUcAGGGAACdTsdT |
| AD-24570.1 | 184 | AGGGcuGccccuGuAGGuudTsdT | 265 | AACCuAcAGGGGcAGCCCUdTsdT |
| AD-24571.1 | 185 | cuGccuGAGAccucAAuAcdTsdT | 266 | GuAUUGAGGUCUcAGGcAGdTsdT |
| AD-24572.1 | 186 | ucAAuAccccAAGuccAccdTsdT | 267 | GGUGGACUUGGGGuAUUGAdTsdT |
| AD-24573.1 | 187 | GAccGAuGGcuucAGuuccdTsdT | 268 | GGAACUGAAGCcAUCGGUCdTsdT |
| AD-24574.1 | 188 | AGuucccuGAAAGAcuAcudTsdT | 269 | AGuAGUCUUUcAGGGAACUdTsdT |
| AD-24575.1 | 189 | cuGccccuGuAGGuuGcuudTsdT | 270 | AAGcAACCuAcAGGGGcAGdTsdT |
| AD-24576.1 | 190 | uGAccGAuGGcuucAGuucdTsdT | 271 | GAACUGAAGCcAUCGGUcAdTsdT |
| AD-24577.1 | 191 | AGAccGccAAGGAuGcAcudTsdT | 272 | AGUGcAUCCUUGGCGGUCUdTsdT |
| AD-24555.1 | 192 | GuAGGuuGcuuAAAAGGGAdTsdT | 273 | UCCCUUUuAAGcAACCuACdTsdT |
| AD-24562.1 | 193 | AcuAcuGGAGcAccGuuAAdTsdT | 274 | UuAACGGUGCUCcAGuAGUdTsdT |
| AD-45078.1 | 194 | GGuGAccGAuGGcuucAGudTsdT | 275 | ACUGAAGCcAUCGGUcACCdTsdT |
| AD-45084.1 | 195 | ccGAuGGcuucAGuucccudTsdT | 276 | AGGGAACUGAAGCcAUCGGdTsdT |
| AD-45090.1 | 196 | GAuGGcuucAGuucccuGAdTsdT | 277 | UcAGGGAACUGAAGCcAUCdTsdT |
| AD-45096.1 | 197 | AuGGcuucAGuucccuGAAdTsdT | 278 | UUcAGGGAACUGAAGCcAUdTsdT |
| AD-45101.1 | 198 | uGcAGccccGGGuAcuccudTsdT | 279 | AGGAGuACCCGGGGCUGcAdTsdT |
| AD-45102.1 | 199 | uGGcuucAGuucccuGAAAdTsdT | 280 | UUUcAGGGAACUGAAGCcAdTsdT |
| AD-45107.1 | 200 | GcAGccccGGGuAcuccuudTsdT | 281 | AAGGAGuACCCGGGGCUGCdTsdT |
| AD-45108.1 | 201 | GcuucAGuucccuGAAAGAdTsdT | 282 | UCUUUcAGGGAACUGAAGCdTsdT |
| AD-45113.1 | 202 | cAAGAccGccAAGGAuGcAdTsdT | 283 | UGcAUCCUUGGCGGUCUUGdTsdT |
| AD-45114.1 | 203 | ucccuGAAAGAcuAcuGGAdTsdT | 284 | UCcAGuAGUCUUUcAGGGAdTsdT |
| AD-45119.1 | 204 | uGGGuGAccGAuGGcuucAdTsdT | 285 | UGAAGCcAUCGGUcACCcAdTsdT |
| AD-45120.1 | 205 | cuGAAAGAcuAcuGGAGcAdTsdT | 286 | UGCUCcAGuAGUCUUUcAGdTsdT |
| AD-45121.1 | 206 | AGAcuAcuGGAGcAccGuudTsdT | 287 | AACGGUGCUCcAGuAGUCUdTsdT |
| AD-45122.1 | 207 | cAGGGcuGccccuGuAGGudTsdT | 288 | ACCuAcAGGGGcAGCCCUGdTsdT |
| AD-45123.1 | 208 | GCfAGCfCfCfCfGGGUfACfUfCfCfUfUfdTsdT | 289 | AAGGAGUfACCCGGGGCUGCdTsdT |
| AD-45124.1 | 209 | GCfUfUfCfAGUfUfCfCfCfUfGAAAGAdTsdT | 290 | UCUUUCfAGGGAACUGAAGCdTsdT |
| AD-45125.1 | 210 | CfUfGAGACfCfUfCfAAUfACfCfCfCfAdTsdT | 291 | UGGGGUfAUUGAGGUCUCfAGdTsdT |
| AD-45126.1 | 211 | GCfUfUfAAAAGGGACfAGUfAUfUfdTsdT | 292 | AAUfACUGUCCCUUUUfAAGCdTsdT |
| AD-45127.1 | 212 | AGcAccGuuAAGGAcAAGudTsdT | 293 | ACUUGUCCUuAACGGUGCUdTsdT |
| AD-45128.1 | 213 | GcuGccccuGuAGGuuGcudTsdT | 294 | AGcAACCuAcAGGGGcAGCdTsdT |
| AD-45129.1 | 214 | CfAAGACfCfGCfCfAAGGAUfGCfAdTsdT | 295 | UGCfAUCCUUGGCGGUCUUGdTsdT |
| AD-45130.1 | 215 | UfCfCfCfUfGAAAGACfUfACfUfGGAdTsdT | 296 | UCCfAGUfAGUCUUUCfAGGGAdTsdT |
| AD-45131.1 | 216 | UfGAGACfCfUfCfAAUfACfCfCfCfAAdTsdT | 297 | UUGGGGUfAUUGAGGUCUCfAdTsdT |
| AD-45132.1 | 217 | CfUfGGACfAAGAAGCfUfGCfUfAUfdTsdT | 298 | AUfAGCfAGCUUCUUGUCCfAGdTsdT |
| AD-45133.1 | 218 | GcAccGuuAAGGAcAAGuudTsdT | 299 | AACUUGUCCUuAACGGUGCdTsdT |
| AD-45135.1 | 219 | UfGGGUfGACfCfGAUfGGCfUfUfCfAdTsdT | 300 | UGAAGCCfAUCGGUCfACCCfAdTsdT |
| AD-45136.1 | 220 | CfUfGAAAGACfUfACfUfGGAGCfAdTsdT | 301 | UGCUCCfAGUfAGUCUUUCfAGdTsdT |
| AD-45137.1 | 221 | CfCfUfCfAAUfACfCfCfCfAAGUfCfCfAdTsdT | 302 | UGGACUUGGGGUfAUUGAGGdTsdT |
| AD-45138.1 | 222 | GGcuGccuGAGAccucAAudTsdT | 303 | AUUGAGGUCUcAGGcAGCCdTsdT |
| AD-45139.1 | 223 | AGGuuGcuuAAAAGGGAcAdTsdT | 304 | UGUCCCUUUuAAGcAACCUdTsdT |
| AD-45140.1 | 224 | GGUfGACfCfGAUfGGCfUfUfCfAGUfdTsdT | 305 | ACUGAAGCCfAUCGGUCfACCdTsdT |
| AD-45141.1 | 225 | AGACfUfACfUfGGAGCfACfCfGUfUfdTsdT | 306 | AACGGUGCUCCfAGUfAGUCUdTsdT |
| AD-45143.1 | 226 | GcuGccuGAGAccucAAuAdTsdT | 307 | uAUUGAGGUCUcAGGcAGCdTsdT |
| AD-45144.1 | 227 | uGcuuAAAAGGGAcAGuAudTsdT | 308 | AuACUGUCCCUUUuAAGcAdTsdT |
| AD-45145.1 | 228 | CfCfGAUfGGCfUfUfCfAGUfUfCfCfCfUfdTsdT | 309 | AGGGAACUGAAGCCfAUCGGdTsdT |
| AD-45146.1 | 229 | AGCfACfCfGUfUfAAGGACfAAGUfdTsdT | 310 | ACUUGUCCUUfAACGGUGCUdTsdT |
| AD-45147.1 | 230 | GCfUfGCfCfCfCfUfGUfAGGUfUfGCfUfdTsdT | 311 | AGCfAACCUfACfAGGGGCfAGCdTsdT |
| AD-45148.1 | 231 | cuGAGAccucAAuAccccAdTsdT | 312 | UGGGGuAUUGAGGUCUcAGdTsdT |
| AD-45149.1 | 232 | GcuuAAAAGGGAcAGuAuudTsdT | 313 | AAuACUGUCCCUUUuAAGCdTsdT |
| AD-45150.1 | 233 | GAUfGGCfUfUfCfAGUfUfCfCfCfUfGAdTsdT | 314 | UCfAGGGAACUGAAGCCfAUCdTsdT |
| AD-45151.1 | 234 | GCfACfCfGUfUfAAGGACfAAGUfUfdTsdT | 315 | AACUUGUCCUUfAACGGUGCdTsdT |
| AD-45152.1 | 235 | CfCfCfUfGUfAGGUfUfGCfUfUfAAAAdTsdT | 316 | UUUUfAAGCfAACCUfACfAGGGdTsdT |
| AD-45153.1 | 236 | uGAGAccucAAuAccccAAdTsdT | 317 | UUGGGGuAUUGAGGUCUcAdTsdT |
| AD-45154.1 | 237 | cuGGAcAAGAAGcuGcuAudTsdT | 318 | AuAGcAGCUUCUUGUCcAGdTsdT |
| AD-45155.1 | 238 | AUfGGCfUfUfCfAGUfUfCfCfCfUfGAAdTsdT | 319 | UUCfAGGGAACUGAAGCCfAUdTsdT |
| AD-45157.1 | 239 | AGGUfUfGCfUfUfAAAAGGGACfAdTsdT | 320 | UGUCCCUUUUfAAGCfAACCUdTsdT |
| AD-45158.1 | 240 | ccucAAuAccccAAGuccAdTsdT | 321 | UGGACUUGGGGuAUUGAGGdTsdT |
| AD-45159.1 | 241 | UfGCfAGCfCfCfCfGGGUfACfUfCfCfUfdTsdT | 322 | AGGAGUfACCCGGGGCUGCfAdTsdT |
| AD-45160.1 | 242 | UfGGCfUfUfCfAGUfUfCfCfCfUfGAAAdTsdT | 323 | UUUCfAGGGAACUGAAGCCfAdTsdT |
| AD-45161.1 | 243 | GCfUfGCfCfUfGAGACfCfUfCfAAUfAdTsdT | 324 | UfAUUGAGGUCUCfAGGCfAGCdTsdT |
| AD-45162.1 | 244 | UfGCfUfUfAAAAGGGACfAGUfAUfdTsdT | 325 | AUfACUGUCCCUUUUfAAGCfAdTsdT |

TABLE 3
ApoC3 modified siRNA (first set) single dose screen

| Duplex ID | 10 nM | 0.1 nM |
|---|---|---|
| AD-24548.1 | 0.06 | 0.38 |
| AD-24549.1 | 0.17 | 0.39 |
| AD-24550.1 | 0.38 | 0.67 |
| AD-24551.1 | 1.08 | 1.02 |
| AD-24552.1 | 0.98 | 0.97 |
| AD-24553.1 | 0.51 | 0.63 |
| AD-24554.1 | 0.63 | 0.78 |
| AD-24555.1 | 0.06 | 0.29 |
| AD-24556.1 | 0.17 | 0.72 |
| AD-24557.1 | 0.81 | 0.93 |
| AD-24558.1 | 0.90 | 0.75 |
| AD-24559.1 | 0.88 | 0.94 |
| AD-24560.1 | 0.75 | 0.85 |
| AD-24561.1 | 0.40 | 0.77 |
| AD-24562.1 | 0.07 | 0.39 |
| AD-24563.1 | 0.55 | 0.91 |
| AD-24564.1 | 0.70 | 1.00 |
| AD-24565.1 | 0.67 | 1.00 |
| AD-24566.1 | 0.97 | 1.01 |
| AD-24567.1 | 0.89 | 0.92 |
| AD-24568.1 | 0.95 | 0.85 |
| AD-24569.1 | 0.68 | 0.88 |
| AD-24570.1 | 0.74 | 0.77 |
| AD-24571.1 | 0.22 | 0.60 |
| AD-24572.1 | 0.92 | 0.91 |
| AD-24573.1 | 0.65 | 0.76 |
| AD-24574.1 | 0.70 | 0.80 |
| AD-24575.1 | 0.63 | 0.94 |
| AD-24576.1 | 0.05 | 0.31 |
| AD-24577.1 | 0.90 | 0.98 |
| AD-45078.1 | 0.38 | 0.78 |
| AD-45084.1 | 0.60 | 0.92 |
| AD-45090.1 | 0.97 | 0.86 |
| AD-45096.1 | 0.47 | 0.82 |
| AD-45101.1 | 1.01 | 1.30 |
| AD-45102.1 | 0.05 | 0.22 |
| AD-45107.1 | 0.87 | 1.06 |
| AD-45108.1 | 0.02 | 0.12 |
| AD-45113.1 | 0.97 | 1.04 |
| AD-45114.1 | 0.37 | 0.77 |
| AD-45119.1 | 0.91 | 0.87 |
| AD-45120.1 | 0.03 | 0.08 |
| AD-45121.1 | 0.93 | 0.94 |
| AD-45122.1 | 0.92 | 0.97 |
| AD-45123.1 | 0.15 | 0.41 |
| AD-45124.1 | 0.03 | 0.07 |
| AD-45125.1 | 0.16 | 0.55 |
| AD-45126.1 | 0.03 | 0.08 |
| AD-45127.1 | 0.58 | 0.75 |
| AD-45128.1 | 0.97 | 0.96 |
| AD-45129.1 | 0.20 | 0.47 |
| AD-45130.1 | 0.05 | 0.12 |
| AD-45131.1 | 0.24 | 0.64 |
| AD-45132.1 | 0.30 | 0.52 |
| AD-45133.1 | 0.03 | 0.10 |
| AD-45135.1 | 0.02 | 0.08 |
| AD-45136.1 | 0.04 | 0.10 |
| AD-45137.1 | 0.02 | 0.19 |
| AD-45138.1 | 0.86 | 1.04 |
| AD-45139.1 | 1.19 | 1.13 |
| AD-45140.1 | 0.07 | 0.33 |
| AD-45141.1 | 0.03 | 0.07 |
| AD-45143.1 | 0.73 | 0.94 |
| AD-45144.1 | 0.45 | 0.95 |
| AD-45145.1 | 0.04 | 0.13 |
| AD-45146.1 | 0.06 | 0.21 |
| AD-45147.1 | 0.20 | 0.49 |
| AD-45148.1 | 0.80 | 0.94 |
| AD-45149.1 | 0.03 | 0.07 |
| AD-45150.1 | 0.09 | 0.28 |
| AD-45151.1 | 0.03 | 0.05 |
| AD-45152.1 | 0.04 | 0.13 |
| AD-45153.1 | 0.92 | 1.02 |
| AD-45154.1 | 0.14 | 0.29 |
| AD-45155.1 | 0.60 | 0.68 |
| AD-45157.1 | 0.13 | 0.29 |
| AD-45158.1 | 0.37 | 0.78 |
| AD-45159.1 | 0.12 | 0.53 |
| AD-45160.1 | 0.03 | 0.11 |
| AD-45161.1 | 0.59 | 0.54 |
| AD-45162.1 | 0.02 | 0.06 |

TABLE 4
ApoC3 modified siRNA (first set) IC50 data

| Duplex Name | IC50 24 hr (nM) | IC50 120 hr (nM) |
|---|---|---|
| AD-24555 | 0.038 | 0.091 |
| AD-24562 | 0.025 | 0.106 |
| AD-24576 | 0.037 | 0.059 |
| AD-45102.1 | 0.012 | 0.022 |
| AD-45108.1 | 0.014 | 0.246 |
| AD-45120.1 | 0.011 | 0.02 |
| AD-45124.1 | 0.013 | 0.264 |
| AD-45126.1 | 0.025 | 0.098 |
| AD-45129.1 | 0.023 | 0.046 |
| AD-45133.1 | 0.014 | 0.015 |
| AD-45135.1 | 0.008 | 0.064 |
| AD-45136.1 | 0.008 | 0.053 |
| AD-45137.1 | 0.010 | 0.077 |
| AD-45141.1 | 0.007 | 0.063 |
| AD-45145.1 | 0.013 | 0.113 |
| AD-45146.1 | 0.031 | 0.316 |
| AD-45149.1 | 0.011 | 0.091 |
| AD-45151.1 | 0.006 | 0.009 |
| AD-45152.1 | 0.011 | 0.051 |
| AD-45160.1 | 0.019 | 0.162 |
| AD-45162.1 | 0.008 | 0.013 |

TABLE 5
ApoC3 modified siRNA (first set) viability
Viability data are expressed as fraction viable relative to
cells treated with the lowest
dose of siRNA (0.0001 nM). 1 = 100% viable, 0 = 100% lethality

| | Fraction viable normalized to low dose | | | | |
|---|---|---|---|---|---|
| | (0.0001 nM) | | | | |
| Conc. (nM) | 10 nM | 1 nM | 0.1 nM | 0.01 nM | 0.0001 nM |
| HeLa day 3 | | | | | |
| AD-45102.1 | 0.57 | 0.72 | 0.96 | 1.06 | 1.00 |
| AD-45108.1 | 0.58 | 0.87 | 0.99 | 0.97 | 1.00 |
| AD-45120.1 | 0.16 | 0.33 | 0.75 | 0.97 | 1.00 |
| AD-45124.1 | 0.69 | 0.84 | 0.96 | 0.94 | 1.00 |
| AD-45126.1 | 0.47 | 0.46 | 0.65 | 0.95 | 1.00 |
| AD-45130.1 | 0.64 | 0.72 | 0.93 | 1.00 | 1.00 |
| AD-45133.1 | 0.22 | 0.51 | 0.94 | 0.94 | 1.00 |
| AD-45151.1 | 0.43 | 0.63 | 1.12 | 1.06 | 1.00 |
| AD-45152.1 | 0.70 | 0.96 | 1.02 | 1.06 | 1.00 |
| AD-45160.1 | 0.44 | 0.68 | 0.83 | 0.99 | 1.00 |
| AD-45162.1 | 0.62 | 0.86 | 1.01 | 1.01 | 1.00 |
| AD-24555 | 0.67 | 0.91 | 1.00 | 0.96 | 1.00 |
| AD-24562 | 0.59 | 0.74 | 0.82 | 0.92 | 1.00 |
| AD-24576 | 0.39 | 0.71 | 1.01 | 0.91 | 1.00 |
| AD-45135.1 | 0.18 | 0.43 | 0.94 | 1.02 | 1.00 |
| AD-45136.1 | 0.33 | 0.48 | 0.86 | 1.00 | 1.00 |
| AD-45137.1 | 0.65 | 0.89 | 0.96 | 0.91 | 1.00 |
| AD-45141.1 | 0.51 | 0.53 | 0.88 | 0.98 | 1.00 |
| AD-45145.1 | 0.33 | 0.58 | 0.95 | 0.92 | 1.00 |
| AD-45146.1 | 0.39 | 0.47 | 0.87 | 0.93 | 1.00 |
| AD-45149.1 | 0.57 | 0.64 | 0.96 | 0.96 | 1.00 |
| AD-1955 | 0.62 | 0.84 | 0.93 | 0.99 | 1.00 |
| PLK | 0.02 | 0.05 | 0.12 | 0.62 | 1.00 |
| AD-19200 | 0.15 | 0.34 | 0.81 | 0.93 | 1.00 |
| HeLa Day 5 | | | | | |
| AD-45102.1 | 0.55 | 0.79 | 0.89 | 1.00 | 1.00 |
| AD-45108.1 | 0.77 | 0.95 | 0.99 | 1.01 | 1.00 |
| AD-45120.1 | 0.06 | 0.28 | 0.90 | 1.00 | 1.00 |
| AD-45124.1 | 1.12 | 1.13 | 1.02 | 1.08 | 1.00 |
| AD-45126.1 | 0.84 | 0.87 | 0.98 | 1.04 | 1.00 |
| AD-45130.1 | 0.50 | 0.81 | 1.04 | 1.11 | 1.00 |
| AD-45133.1 | 0.01 | 0.11 | 0.76 | 0.94 | 1.00 |
| AD-45151.1 | 0.17 | 0.41 | 0.63 | 1.00 | 1.00 |
| AD-45152.1 | 0.82 | 0.97 | 0.84 | 1.01 | 1.00 |
| AD-45160.1 | 0.47 | 0.83 | 0.94 | 1.03 | 1.00 |
| AD-45162.1 | 0.79 | 0.94 | 0.83 | 1.00 | 1.00 |
| AD-24555 | 0.92 | 1.04 | 0.99 | 0.99 | 1.00 |
| AD-24562 | 0.71 | 0.98 | 1.05 | 1.03 | 1.00 |
| AD-24576 | 0.10 | 0.59 | 0.80 | 1.00 | 1.00 |
| AD-45135.1 | 0.04 | 0.66 | 1.02 | 1.02 | 1.00 |
| AD-45136.1 | 0.23 | 0.67 | 1.06 | 0.96 | 1.00 |
| AD-45137.1 | 0.73 | 0.93 | 1.02 | 0.98 | 1.00 |
| AD-45141.1 | 0.30 | 0.51 | 0.91 | 0.97 | 1.00 |
| AD-45145.1 | 0.27 | 0.76 | 1.01 | 1.01 | 1.00 |
| AD-45146.1 | 0.29 | 0.59 | 0.98 | 1.02 | 1.00 |
| AD-45149.1 | 0.71 | 0.84 | 1.01 | 0.99 | 1.00 |
| AD-1955 | 0.67 | 0.89 | 0.92 | 0.95 | 1.00 |
| PLK | −0.03 | 0.02 | 0.06 | 0.47 | 0.88 |
| AD-19200 | 0.05 | 0.49 | 1.01 | 1.03 | 1.00 |
| Hep3B Day 3 | | | | | |
| AD-45102.1 | 0.84 | 1.09 | 1.02 | 1.06 | 1.00 |
| AD-45108.1 | 0.88 | 1.02 | 0.99 | 0.96 | 1.00 |
| AD-45120.1 | 0.69 | 0.99 | 0.99 | 0.94 | 1.00 |
| AD-45124.1 | 0.86 | 1.09 | 0.95 | 0.92 | 1.00 |
| AD-45126.1 | 0.73 | 0.95 | 0.99 | 0.97 | 1.00 |
| AD-45130.1 | 0.81 | 1.00 | 1.04 | 1.00 | 1.00 |
| AD-45133.1 | 0.64 | 0.98 | 1.05 | 1.02 | 1.00 |
| AD-45151.1 | 0.53 | 0.70 | 0.91 | 0.86 | 1.00 |
| AD-45152.1 | 0.86 | 0.93 | 0.98 | 1.02 | 1.00 |
| AD-45160.1 | 1.03 | 1.11 | 1.00 | 0.95 | 1.00 |
| AD-45162.1 | 0.91 | 0.95 | 1.02 | 0.96 | 1.00 |
| AD-24555 | 0.83 | 0.82 | 0.93 | 0.81 | 1.00 |
| AD-24562 | 1.14 | 1.26 | 1.15 | 1.03 | 1.00 |
| AD-24576 | 0.84 | 1.06 | 1.11 | 1.00 | 1.00 |
| AD-45135.1 | 0.99 | 1.18 | 1.17 | 1.18 | 1.00 |
| AD-45136.1 | 0.83 | 0.98 | 1.05 | 1.12 | 1.00 |
| AD-45137.1 | 0.93 | 1.12 | 1.04 | 1.03 | 1.00 |
| AD-45141.1 | 0.71 | 0.89 | 0.93 | 1.12 | 1.00 |
| AD-45145.1 | 0.87 | 1.07 | 1.03 | 1.05 | 1.00 |
| AD-45146.1 | 0.85 | 1.01 | 1.07 | 1.09 | 1.00 |
| AD-45149.1 | 0.98 | 1.20 | 1.10 | 1.04 | 1.00 |
| AD-1955 | 0.62 | 0.92 | 0.95 | 0.93 | 1.00 |
| PLK | 0.21 | 0.32 | 0.47 | 0.82 | 1.00 |
| AD-19200 | 0.25 | 0.63 | 1.03 | 1.01 | 1.00 |
| Hep3B Day 5 | | | | | |
| AD-45102.1 | 0.73 | 0.96 | 1.03 | 0.94 | 1.00 |
| AD-45108.1 | 1.01 | 0.83 | 0.96 | 0.96 | 1.00 |
| AD-45120.1 | 0.30 | 0.47 | 0.81 | 1.00 | 1.00 |
| AD-45124.1 | 1.33 | 1.24 | 0.89 | 1.04 | 1.00 |
| AD-45126.1 | 1.08 | 1.05 | 1.00 | 0.92 | 1.00 |
| AD-45130.1 | 0.86 | 0.92 | 1.09 | 0.93 | 1.00 |
| AD-45133.1 | 0.47 | 0.58 | 0.93 | 0.95 | 1.00 |
| AD-45151.1 | 0.29 | 0.57 | 0.93 | 0.91 | 1.00 |
| AD-45152.1 | 1.00 | 0.94 | 0.93 | 0.96 | 1.00 |
| AD-45160.1 | 1.46 | 1.25 | 1.20 | 0.90 | 1.00 |
| AD-45162.1 | 0.83 | 0.84 | 0.89 | 0.85 | 1.00 |
| AD-24555 | 1.13 | 1.00 | 0.99 | 0.83 | 1.00 |
| AD-24562 | 1.16 | 1.13 | 1.03 | 0.97 | 1.00 |
| AD-24576 | 0.68 | 0.92 | 1.04 | 0.90 | 1.00 |
| AD-45135.1 | 0.81 | 1.23 | 1.35 | 1.19 | 1.00 |
| AD-45136.1 | 0.37 | 0.74 | 0.92 | 1.00 | 1.00 |
| AD-45137.1 | 0.74 | 0.90 | 0.99 | 0.96 | 1.00 |
| AD-45141.1 | 0.32 | 0.43 | 0.61 | 0.96 | 1.00 |
| AD-45145.1 | 0.52 | 0.74 | 0.96 | 1.00 | 1.00 |
| AD-45146.1 | 0.60 | 0.57 | 0.86 | 1.02 | 1.00 |
| AD-45149.1 | 0.83 | 0.94 | 1.01 | 0.97 | 1.00 |
| AD-1955 | 0.63 | 0.74 | 0.93 | 0.85 | 1.00 |
| PLK | 0.03 | 0.12 | 0.29 | 0.86 | 1.00 |
| AD-19200 | −0.04 | 0.41 | 0.84 | 0.95 | 1.00 |

TABLE 6
NpoC3 siRNA (second set) unmodified sequences and duplex names of modified siRNA

| | SEQ | | SEQ | | Modified | Modifi- | |
|---|---|---|---|---|---|---|---|
| Unmodified | ID | Unmodified | ID | Unmodified | duplex | cation | Position in |
| duplex name | NO | sense | NO | antisense | name | Type | NM_000040.1 |
| AD-45101.1UM | 326 | UGCAGCCCCGGGUACUCCU | 353 | AGGAGUACCCGGGGCUGCA | AD-46822.1 | end | 48-66 |
| | | | | | AD-47334.1 | FOME | 48-66 |
| | | | | | AD-47361.1 | DECAF | 48-66 |
| AD-45107.1UM | 327 | GCAGCCCCGGGUACUCCUU | 354 | AAGGAGUACCCGGGGCUGC | AD-46825.1 | UMdTsdT | 49-67 |
| | | | | | AD-47338.1 | FOME | 49-67 |
| | | | | | AD-47365.1 | DECAF | 49-67 |
| AD-45113.1UM | 328 | CAAGACCGCCAAGGAUGCA | 355 | UGCAUCCUUGGCGGUCUUG | AD-46828.1 | UMdTsdT | 166-184 |
| | | | | | AD-47342.1 | FOME | 166-184 |
| | | | | | AD-47369.1 | DECAF | 166-184 |
| AD-45119.1UM | 329 | UGGGUGACCGAUGGCUUCA | 356 | UGAAGCCAUCGGUCACCCA | AD-46831.1 | UMdTsdT | 230-248 |
| | | | | | AD-47346.1 | FOME | 230-248 |
| | | | | | AD-47373.1 | DECAF | 230-248 |
| AD-45078.1UM | 330 | GGUGACCGAUGGCUUCAGU | 357 | ACUGAAGCCAUCGGUCACC | AD-46811.1 | UMdTsdT | 232-250 |
| | | | | | AD-47349.1 | FOME | 232-250 |
| | | | | | AD-47376.1 | DECAF | 232-250 |
| AD-45084.1UM | 331 | CCGAUGGCUUCAGUUCCCU | 358 | AGGGAACUGAAGCCAUCGG | AD-46815.1 | UMdTsdT | 237-255 |
| | | | | | AD-47352.1 | FOME | 237-255 |
| | | | | | AD-47379.1 | DECAF | 237-255 |
| AD-45090.1UM | 332 | GAUGGCUUCAGUUCCCUGA | 359 | UCAGGGAACUGAAGCCAUC | AD-46818.1 | UMdTsdT | 239-257 |
| | | | | | AD-47355.1 | FOME | 239-257 |
| | | | | | AD-47382.1 | DECAF | 239-257 |
| AD-45096.1UM | 333 | AUGGCUUCAGUUCCCUGAA | 360 | UUCAGGGAACUGAAGCCAU | AD-46820.1 | UMdTsdT | 240-258 |
| | | | | | AD-47358.1 | FOME | 240-258 |
| | | | | | AD-47385.1 | DECAF | 240-258 |
| AD-45102.1UM | 334 | UGGCUUCAGUUCCCUGAAA | 361 | UUUCAGGGAACUGAAGCCA | AD-46823.1 | UMdTsdT | 241-259 |
| | | | | | AD-47335.1 | FOME | 241-259 |
| | | | | | AD-47362.1 | DECAF | 241-259 |
| AD-45108.1UM | 335 | GCUUCAGUUCCCUGAAAGA | 362 | UCUUUCAGGGAACUGAAGC | AD-46826.1 | UMdTsdT | 243-261 |
| | | | | | AD-47339.1 | FOME | 243-261 |
| | | | | | AD-47366.1 | DECAF | 243-261 |
| AD-45120.1UM | 336 | CUGAAAGACUACUGGAGCA | 363 | UGCUCCAGUAGUCUUUCAG | AD-46829.1 | UMdTsdT | 254-272 |
| | | | | | AD-47347.1 | FOME | 254-272 |
| | | | | | AD-47374.1 | DECAF | 254-272 |
| AD-45127.1UM | 337 | AGCACCGUUAAGGACAAGU | 364 | ACUUGUCCUUAACGGUGCU | AD-46832.1 | UMdTsdT | 269-287 |
| | | | | | AD-47353.1 | FOME | 269-287 |
| | | | | | AD-47380.1 | DECAF | 269-287 |
| AD-45133.1UM | 338 | GCACCGUUAAGGACAAGUU | 365 | AACUUGUCCUUAACGGUGC | AD-46812.1 | UMdTsdT | 270-288 |
| | | | | | AD-47356.1 | FOME | 270-288 |
| | | | | | AD-47383.1 | DECAF | 270-288 |
| AD-45143.1UM | 339 | GCUGCCUGAGACCUCAAUA | 366 | UAUUGAGGUCUCAGGCAGC | AD-46816.1 | UMdTsdT | 338-356 |
| | | | | | AD-47336.1 | FOME | 338-356 |
| | | | | | AD-47363.1 | DECAF | 338-356 |
| AD-45148.1UM | 340 | CUGAGACCUCAAUACCCCA | 367 | UGGGGUAUUGAGGUCUCAG | AD-46819.1 | UMdTsdT | 343-361 |
| | | | | | AD-47340.1 | FOME | 343-361 |
| | | | | | AD-47367.1 | DECAF | 343-361 |
| AD-45153.1UM | 341 | UGAGACCUCAAUACCCCAA | 368 | UUGGGGUAUUGAGGUCUCA | AD-46821.1 | UMdTsdT | 344-362 |
| | | | | | AD-47344.1 | FOME | 344-362 |
| | | | | | AD-47371.1 | DECAF | 344-362 |
| AD-45158.1UM | 342 | CCUCAAUACCCCAAGUCCA | 369 | UGGACUUGGGGUAUUGAGG | AD-46824.1 | UMdTsdT | 349-367 |
| | | | | | AD-47348.1 | FOME | 349-367 |
| | | | | | AD-47375.1 | DECAF | 349-367 |
| AD-45128.1UM | 343 | GCUGCCCCUGUAGGUUGCU | 370 | AGCAACCUACAGGGGCAGC | AD-46827.1 | UMdTsdT | 414-432 |
| | | | | | AD-47354.1 | FOME | 414-432 |
| AD-45139.1UM | 344 | AGGUUGCUUAAAAGGGACA | 371 | UGUCCCUUUUAAGCAACCU | AD-46830.1 | UMdTsdT | 425-443 |
| | | | | | AD-47360.1 | FOME | 425-443 |
| | | | | | AD-47387.1 | DECAF | 425-443 |
| AD-45144.1UM | 345 | UGCUUAAAAGGGACAGUAU | 372 | AUACUGUCCCUUUUAAGCA | AD-46833.1 | UMdTsdT | 429-447 |
| | | | | | AD-47337.1 | FOME | 429-447 |
| | | | | | AD-47364.1 | DECAF | 429-447 |
| AD-45149.1UM | 346 | GCUUAAAAGGGACAGUAUU | 373 | AAUACUGUCCCUUUUAAGC | AD-46813.1 | UMdTsdT | 430-448 |
| | | | | | AD-47341.1 | FOME | 430-448 |
| | | | | | AD-47368.1 | DECAF | 430-448 |
| AD-45154.1UM | 347 | CUGGACAAGAAGCUGCUAU | 374 | AUAGCAGCUUCUUGUCCAG | AD-46817.1 | UMdTsdT | 514-532 |
| | | | | | AD-47345.1 | FOME | 514-532 |
| | | | | | AD-47372.1 | DECAF | 514-532 |
| AD-45114.1UM | 348 | UCCCUGAAAGACUACUGGA | 375 | UCCAGUAGUCUUUCAGGGA | AD-47343.1 | FOME | 251-269 |
| AD-45141.1UM | 349 | AGACUACUGGAGCACCGUU | 376 | AACGGUGCUCCAGUAGUCU | AD-47350.1 | FOME | 259-277 |
| AD-45138.1UM | 350 | GGCUGCCUGAGACCUCAAU | 377 | AUUGAGGUCUCAGGCAGCC | AD-47359.1 | FOME | 337-355 |
| | | | | | AD-47386.1 | DECAF | 337-355 |
| AD-45122.1UM | 351 | CAGGGCUGCCCCUGUAGGU | 378 | ACCUACAGGGGCAGCCCUG | AD-47351.1 | FOME | 410-428 |
| | | | | | AD-47378.1 | DECAF | 410-428 |
| AD-45152.1UM | 352 | CCCUGUAGGUUGCUUAAAA | 379 | UUUUAAGCAACCUACAGGG | AD-47357.1 | FOME | 419-437 |
| | | | | | AD-47357.1 | FOME | 419-437 |

TABLE 7
ANC3 modified siRNA (second set) sequences

| | SEQ | | SEQ | |
|---|---|---|---|---|
| Modified | ID | Sense strand sequence | ID | Antisense strand sequence |
| duplex name | NO: | 5′ to 3′ | NO: | 5′ to 3′ |
| AD-46822.1 | 380 | UGCAGCCCCGGGUACUCCUdTsdT | 453 | AGGAGUACCCGGGGCUGCAdTsdT |
| AD-47334.1 | 381 | UfgCfaGfcCfcCfgGfgUfaCfuCfcUfdTsdT | 454 | aGfgAfgUfaCfcCfgGfgGfcUfgCfadTsdT |
| AD-47361.1 | 382 | uGcAGccccGGGuAcuccudTsdT | 455 | AGGAGuACCCGGGGCugcadTsdT |
| AD-46825.1 | 383 | GCAGCCCCGGGUACUCCUUdTsdT | 456 | AAGGAGUACCCGGGGCUGCdTsdT |
| AD-47338.1 | 384 | GfcAfgCfcCfcGfgGfuAfcUfcCfuUfdTsdT | 457 | aAfgGfaGfuAfcCfcGfgGfgCfuGfcdTsdT |
| AD-47365.1 | 385 | GcAGccccGGGuAcuccuudTsdT | 458 | AAGGAGuACCCGGGGCugcdTsdT |
| AD-46828.1 | 386 | CAAGACCGCCAAGGAUGCAdTsdT | 459 | UGCAUCCUUGGCGGUCUUGdTsdT |
| AD-47342.1 | 387 | CfaAfgAfcCfgCfcAfaGfgAfuGfcAfdTsdT | 460 | uGfcAfuCfcUfuGfgCfgGfuCfuUfgdTsdT |
| AD-47369.1 | 388 | cAAGAccGccAAGGAuGcAdTsdT | 461 | UGCAUCCuUGGCGGuCuugdTsdT |
| AD-46831.1 | 389 | UGGGUGACCGAUGGCUUCAdTsdT | 462 | UGAAGCCAUCGGUCACCCAdTsdT |
| AD-47346.1 | 390 | UfgGfgUfgAfcCfgAfuGfgCfuUfcAfdTsdT | 463 | uGfaAfgCfcAfuCfgGfuCfaCfcCfadTsdT |
| AD-47373.1 | 391 | uGGGuGAccGAuGGcuucAdTsdT | 464 | UGAAGCCAUCGGuCACccadTsdT |
| AD-46811.1 | 392 | GGUGACCGAUGGCUUCAGUdTsdT | 465 | ACUGAAGCCAUCGGUCACCdTsdT |
| AD-47349.1 | 393 | GfgUfgAfcCfgAfuGfgCfuUfcAfgUfdTsdT | 466 | aCfuGfaAfgCfcAfuCfgGfuCfaCfcdTsdT |
| AD-47376.1 | 394 | GGuGAccGAuGGcuucAGudTsdT | 467 | ACuGAAGCCAuCGGuCaccdTsdT |
| AD-46815.1 | 395 | CCGAUGGCUUCAGUUCCCUdTsdT | 468 | AGGGAACUGAAGCCAUCGGdTsdT |
| AD-47352.1 | 396 | CfcGfaUfgGfcUfuCfaGfuUfcCfcUfdTsdT | 469 | aGfgGfaAfcUfgAfaGfcCfaUfcGfgdTsdT |
| AD-47379.1 | 397 | ccGAuGGcuucAGuucccudTsdT | 470 | AGGGAACuGAAGCCAucggdTsdT |
| AD-46818.1 | 398 | GAUGGCUUCAGUUCCCUGAdTsdT | 471 | UCAGGGAACUGAAGCCAUCdTsdT |
| AD-47355.1 | 399 | GfaUfgGfcUfuCfaGfuUfcCfcUfgAfdTsdT | 472 | uCfaGfgGfaAfcUfgAfaGfcCfaUfcdTsdT |
| AD-47382.1 | 400 | GAuGGcuucAGuucccuGAdTsdT | 473 | UCAGGGAACuGAAGCCaucdTsdT |
| AD-46820.1 | 401 | AUGGCUUCAGUUCCCUGAAdTsdT | 474 | UUCAGGGAACUGAAGCCAUdTsdT |
| AD-47358.1 | 402 | AfuGfgCfuUfcAfgUfuCfcCfuGfaAfdTsdT | 475 | uUfcAfgGfgAfaCfuGfaAfgCfcAfudTsdT |
| AD-47385.1 | 403 | AuGGcuucAGuucccuGAAdTsdT | 476 | UUCAGGGAACuGAAGCcaudTsdT |
| AD-46823.1 | 404 | UGGCUUCAGUUCCCUGAAAdTsdT | 477 | UUUCAGGGAACUGAAGCCAdTsdT |
| AD-47335.1 | 405 | UfgGfcUfuCfaGfuUfcCfcUfgAfaAfdTsdT | 478 | uUfuCfaGfgGfaAfcUfgAfaGfcCfadTsdT |
| AD-47362.1 | 406 | uGGcuucAGuucccuGAAAdTsdT | 479 | UuUCAGGGAACuGAAGccadTsdT |
| AD-46826.1 | 407 | GCUUCAGUUCCCUGAAAGAdTsdT | 480 | UCUUUCAGGGAACUGAAGCdTsdT |
| AD-47339.1 | 408 | GfcUfuCfaGfuUfcCfcUfgAfaAfgAfdTsdT | 481 | uCfuUfuCfaGfgGfaAfcUfgAfaGfcdTsdT |
| AD-47366.1 | 409 | GcuucAGuucccuGAAAGAdTsdT | 482 | UCuUUCAGGGAACuGAagcdTsdT |
| AD-46829.1 | 410 | CUGAAAGACUACUGGAGCAdTsdT | 483 | UGCUCCAGUAGUCUUUCAGdTsdT |
| AD-47347.1 | 411 | CfuGfaAfaGfaCfuAfcUfgGfaGfcAfdTsdT | 484 | uGfcUfcCfaGfuAfgUfcUfuUfcAfgdTsdT |
| AD-47374.1 | 412 | cuGAAAGAcuAcuGGAGcAdTsdT | 485 | UGCUCCAGuAGuCuuucagdTsdT |
| AD-46832.1 | 413 | AGCACCGUUAAGGACAAGUdTsdT | 486 | ACUUGUCCUUAACGGUGCUdTsdT |
| AD-47353.1 | 414 | AfgCfaCfcGfuUfaAfgGfaCfaAfgUfdTsdT | 487 | aCfuUfgUfcCfuUfaAfcGfgUfgCfudTsdT |
| AD-47380.1 | 415 | AGcAccGuuAAGGAcAAGudTsdT | 488 | ACuUGUCCuUAACGGugcudTsdT |
| AD-46812.1 | 416 | GCACCGUUAAGGACAAGUUdTsdT | 489 | AACUUGUCCUUAACGGUGCdTsdT |
| AD-47356.1 | 417 | GfcAfcCfgUfuAfaGfgAfcAfaGfuUfdTsdT | 490 | aAfcUfuGfuCfcUfuAfaCfgGfuGfcdTsdT |
| AD-47383.1 | 418 | GcAccGuuAAGGAcAAGuudTsdT | 491 | AACuUGUCCuuAACGGugcdTsdT |
| AD-46816.1 | 419 | GCUGCCUGAGACCUCAAUAdTsdT | 492 | UAUUGAGGUCUCAGGCAGCdTsdT |
| AD-47336.1 | 420 | GfcUfgCfcUfgAfgAfcCfuCfaAfuAfdTsdT | 493 | uAfuUfgAfgGfuCfuCfaGfgCfaGfcdTsdT |
| AD-47363.1 | 421 | GcuGccuGAGAccucAAuAdTsdT | 494 | UAuUGAGGUCuCAGGCagcdTsdT |
| AD-46819.1 | 422 | CUGAGACCUCAAUACCCCAdTsdT | 495 | UGGGGUAUUGAGGUCUCAGdTsdT |
| AD-47340.1 | 423 | CfuGfaGfaCfcUfcAfaUfaCfcCfcAfdTsdT | 496 | uGfgGfgUfaUfuGfaGfgUfcUfcAfgdTsdT |
| AD-47367.1 | 424 | cuGAGAccucAAuAccccAdTsdT | 497 | UGGGGuAuUGAGGuCucagdTsdT |
| AD-46821.1 | 425 | UGAGACCUCAAUACCCCAAdTsdT | 498 | UUGGGGUAUUGAGGUCUCAdTsdT |
| AD-47344.1 | 426 | UfgAfgAfcCfuCfaAfuAfcCfcCfaAfdTsdT | 499 | uUfgGfgGfuAfuUfgAfgGfuCfuCfadTsdT |
| AD-47371.1 | 427 | uGAGAccucAAuAccccAAdTsdT | 500 | UuGGGGuAuUGAGGuCucadTsdT |
| AD-46824.1 | 428 | CCUCAAUACCCCAAGUCCAdTsdT | 501 | UGGACUUGGGGUAUUGAGGdTsdT |
| AD-47348.1 | 429 | CfcUfcAfaUfaCfcCfcAfaGfuCfcAfdTsdT | 502 | uGfgAfcUfuGfgGfgUfaUfuGfaGfgdTsdT |
| AD-47375.1 | 430 | ccucAAuAccccAAGuccAdTsdT | 503 | UGGACuUGGGGuAuuGaggdTsdT |
| AD-46827.1 | 431 | GCUGCCCCUGUAGGUUGCUdTsdT | 504 | AGCAACCUACAGGGGCAGCdTsdT |
| AD-47354.1 | 432 | GfcUfgCfcCfcUfgUfaGfgUfuGfcUfdTsdT | 505 | aGfcAfaCfcUfaCfaGfgGfgCfaGfcdTsdT |
| AD-46830.1 | 433 | AGGUUGCUUAAAAGGGACAdTsdT | 506 | UGUCCCUUUUAAGCAACCUdTsdT |
| AD-47360.1 | 434 | AfgGfuUfgCfuUfaAfaAfgGfgAfcAfdTsdT | 507 | uGfuCfcCfuUfuUfaAfgCfaAfcCfudTsdT |
| AD-47387.1 | 435 | AGGuuGcuuAAAAGGGAcAdTsdT | 508 | UGUCCCuUuUAAGCAAccudTsdT |
| AD-46833.1 | 436 | UGCUUAAAAGGGACAGUAUdTsdT | 509 | AUACUGUCCCUUUUAAGCAdTsdT |
| AD-47337.1 | 437 | UfgCfuUfaAfaAfgGfgAfcAfgUfaUfdTsdT | 510 | aUfaCfuGfuCfcCfuUfuUfaAfgCfadTsdT |
| AD-47364.1 | 438 | uGcuuAAAAGGGAcAGuAudTsdT | 511 | AuACuGUCCCuuuuAAgcadTsdT |
| AD-46813.1 | 439 | GCUUAAAAGGGACAGUAUUdTsdT | 512 | AAUACUGUCCCUUUUAAGCdTsdT |
| AD-47341.1 | 440 | GfcUfuAfaAfaGfgGfaCfaGfuAfuUfdTsdT | 513 | aAfuAfcUfgUfcCfcUfuUfuAfaGfcdTsdT |
| AD-47368.1 | 441 | GcuuAAAAGGGAcAGuAuudTsdT | 514 | AAuACuGUCCCuuuuAagcdTsdT |
| AD-46817.1 | 442 | CUGGACAAGAAGCUGCUAUdTsdT | 515 | AUAGCAGCUUCUUGUCCAGdTsdT |
| AD-47345.1 | 443 | CfuGfgAfcAfaGfaAfgCfuGfcUfaUfdTsdT | 516 | aUfaGfcAfgCfuUfcUfuGfuCfcAfgdTsdT |
| AD-47372.1 | 444 | cuGGAcAAGAAGcuGcuAudTsdT | 517 | AuAGCAGCuUCuuGuCcagdTsdT |
| AD-47343.1 | 445 | UfcCfcUfgAfaAfgAfcUfaCfuGfgAfdTsdT | 518 | uCfcAfgUfaGfuCfuUfuCfaGfgGfadTsdT |
| AD-47350.1 | 446 | AfgAfcUfaCfuGfgAfgCfaCfcGfuUfdTsdT | 519 | aAfcGfgUfgCfuCfcAfgUfaGfuCfudTsdT |
| AD-47359.1 | 447 | GfgCfuGfcCfuGfaGfaCfcUfcAfaUfdTsdT | 520 | aUfuGfaGfgUfcUfcAfgGfcAfgCfcdTsdT |
| AD-47386.1 | 448 | GGcuGccuGAGAccucAAudTsdT | 521 | AuUGAGGUCuCAGGCAgccdTsdT |
| AD-47351.1 | 449 | CfaGfgGfcUfgCfcCfcUfgUfaGfgUfdTsdT | 522 | aCfcUfaCfaGfgGfgCfaGfcCfcUfgdTsdT |
| AD-47378.1 | 450 | cAGGGcuGccccuGuAGGudTsdT | 523 | ACCuACAGGGGCAGCCcugdTsdT |
| AD-47357.1 | 451 | CfcCfuGfuAfgGfuUfgCfuUfaAfaAfdTsdT | 524 | uUfuUfaAfgCfaAfcCfuAfcAfgGfgdTsdT |
| AD-47357.1 | 452 | CfcCfuGfuAfgGfuUfgCfuUfaAfaAfdTsdT | 525 | uUfuUfaAfgCfaAfcCfuAfcAfgGfgdTsdT |

TABLE 8
ApoC3 modified siRNA (second set) Activity in cells

| | | | 10 nM | 0.1 nM |
|---|---|---|---|---|
| duplex Name | 10 nM | 0.1 nM | SD | SD |
| AD-46822.1 | 0.15 | 0.31 | 0.00 | 0.00 |
| AD-47334.1 | 0.41 | 0.82 | 0.12 | 0.10 |
| AD-47361.1 | 0.89 | 1.00 | 0.19 | 0.03 |
| AD-46825.1 | 0.13 | 0.26 | 0.01 | 0.01 |
| AD-47338.1 | 0.38 | 0.73 | 0.12 | 0.12 |
| AD-47365.1 | 0.74 | 0.93 | 0.01 | 0.07 |
| AD-46828.1 | 0.15 | 0.57 | 0.00 | 0.04 |
| AD-47342.1 | 0.38 | 0.91 | 0.26 | 0.06 |
| AD-47369.1 | 1.10 | 1.26 | 0.18 | 0.29 |
| AD-46831.1 | 0.01 | 0.37 | 0.00 | 0.05 |
| AD-47346.1 | 0.57 | 0.95 | 0.17 | 0.08 |
| AD-47373.1 | 0.80 | 1.06 | 0.06 | 0.15 |
| AD-46811.1 | 0.03 | 0.31 | 0.01 | 0.04 |
| AD-47349.1 | 0.03 | 0.29 | 0.02 | 0.18 |
| AD-47376.1 | 0.38 | 0.95 | 0.15 | 0.01 |
| AD-46815.1 | 0.06 | 0.37 | 0.00 | 0.00 |
| AD-47352.1 | 0.04 | 0.35 | 0.04 | 0.23 |
| AD-47379.1 | 0.81 | 1.03 | 0.12 | 0.05 |
| AD-46818.1 | 0.03 | 0.26 | 0.00 | 0.03 |
| AD-47355.1 | 0.23 | 0.60 | 0.14 | 0.08 |
| AD-47382.1 | 0.40 | 0.81 | 0.11 | 0.09 |
| AD-46820.1 | 0.03 | 0.38 | 0.00 | 0.02 |
| AD-47358.1 | 0.05 | 0.45 | 0.03 | 0.31 |
| AD-47385.1 | 0.53 | 0.84 | 0.12 | 0.19 |
| AD-46823.1 | 0.02 | 0.22 | 0.00 | 0.04 |
| AD-47335.1 | 0.15 | 0.70 | 0.05 | 0.00 |
| AD-47362.1 | 0.66 | 1.07 | 0.01 | 0.09 |
| AD-46826.1 | 0.02 | 0.18 | 0.00 | 0.02 |
| AD-47339.1 | 0.19 | 0.62 | 0.12 | 0.04 |
| AD-47366.1 | 0.60 | 0.82 | 0.02 | 0.09 |
| AD-46829.1 | 0.02 | 0.22 | 0.01 | 0.01 |
| AD-47347.1 | 0.16 | 0.66 | 0.14 | 0.11 |
| AD-47374.1 | 0.90 | 1.15 | 0.03 | 0.03 |
| AD-46832.1 | 0.09 | 0.56 | 0.02 | 0.01 |
| AD-47353.1 | 0.21 | 0.65 | 0.04 | 0.02 |
| AD-47380.1 | 0.66 | 1.02 | 0.07 | 0.02 |
| AD-46812.1 | 0.01 | 0.10 | 0.00 | 0.00 |
| AD-47356.1 | 0.02 | 0.13 | 0.01 | 0.13 |
| AD-47383.1 | 0.03 | 0.21 | 0.02 | 0.10 |
| AD-46816.1 | 0.04 | 0.53 | 0.00 | 0.01 |
| AD-47336.1 | 0.10 | 0.37 | 0.06 | 0.14 |
| AD-47363.1 | 0.54 | 0.88 | 0.03 | 0.14 |
| AD-46819.1 | 0.06 | 0.49 | 0.01 | 0.02 |
| AD-47340.1 | 0.20 | 0.72 | 0.18 | 0.21 |
| AD-47367.1 | 0.99 | 1.10 | 0.17 | 0.07 |
| AD-46821.1 | 0.19 | 0.67 | 0.01 | 0.01 |
| AD-47344.1 | 0.48 | 0.90 | 0.16 | 0.06 |
| AD-47371.1 | 1.01 | 0.92 | 0.14 | 0.12 |
| AD-46824.1 | 0.02 | 0.21 | 0.00 | 0.02 |
| AD-47348.1 | 0.19 | 0.66 | 0.20 | 0.24 |
| AD-47375.1 | 0.83 | 0.94 | 0.06 | 0.00 |
| AD-46827.1 | 0.05 | 0.54 | 0.01 | 0.06 |
| AD-47354.1 | 0.23 | 0.79 | 0.15 | 0.19 |
| AD-46830.1 | 0.64 | 1.01 | 0.03 | 0.00 |
| AD-47360.1 | 0.76 | 1.22 | 0.09 | 0.22 |
| AD-47387.1 | 0.90 | 0.84 | 0.04 | 0.09 |
| AD-46833.1 | 0.06 | 0.46 | 0.01 | 0.05 |
| AD-47337.1 | 0.05 | 0.23 | 0.03 | 0.16 |
| AD-47364.1 | 0.52 | 0.79 | 0.10 | 0.02 |
| AD-46813.1 | 0.02 | 0.27 | 0.00 | 0.01 |
| AD-47341.1 | 0.04 | 0.17 | 0.02 | 0.11 |
| AD-47368.1 | 0.46 | 0.56 | 0.02 | 0.13 |
| AD-46817.1 | 0.10 | 0.29 | 0.01 | 0.04 |
| AD-47345.1 | 0.27 | 0.58 | 0.17 | 0.19 |
| AD-47372.1 | 0.34 | 0.44 | 0.17 | 0.04 |
| AD-47343.1 | 0.21 | 0.66 | 0.15 | 0.18 |
| AD-47350.1 | 0.12 | 0.58 | 0.05 | 0.31 |
| AD-47359.1 | 0.82 | 1.04 | 0.10 | 0.18 |
| AD-47386.1 | 0.88 | 1.28 | 0.15 | 0.21 |
| AD-47351.1 | 0.48 | 1.01 | 0.18 | 0.14 |
| AD-47378.1 | 1.10 | 1.08 | 0.09 | 0.03 |
| AD-47357.1 | 0.09 | 0.28 | 0.02 | 0.25 |
| AD-47357.1 | 0.09 | 0.28 | 0.02 | 0.25 |

TABLE 9
ApoC3 siRNA (third set) duplex names
and modification patterns of modified siRNA

| DuplexName | Chemistry |
|---|---|
| AD-45101.1end | Endolight |
| AD-45159.1 | Fluorolight |
| AD-46822.1 | UMdTsdT |
| AD-47334.1 | FOME |
| AD-47361.1 | DECAF |
| AD-45107.1end | Endolight |
| AD-45123.1 | Fluorolight |
| AD-46825.1 | UMdTsdT |
| AD-47338.1 | FOME |
| AD-47365.1 | DECAF |
| AD-45113.1end | Endolight |
| AD-45129.1 | Fluorolight |
| AD-46828.1 | UMdTsdT |
| AD-47342.1 | FOME |
| AD-47369.1 | DECAF |
| AD-45119.1end | Endolight |
| AD-45135.1 | Fluorolight |
| AD-46831.1 | UMdTsdT |
| AD-47346.1 | FOME |
| AD-47373.1 | DECAF |
| AD-45078.1end | Endolight |
| AD-45140.1 | Fluorolight |
| AD-46811.1 | UMdTsdT |
| AD-47349.1 | FOME |
| AD-47376.1 | DECAF |
| AD-45084.1end | Endolight |
| AD-45145.1 | Fluorolight |
| AD-46815.1 | UMdTsdT |
| AD-47352.1 | FOME |
| AD-47379.1 | DECAF |
| AD-45090.1end | Endolight |
| AD-45150.1 | Fluorolight |
| AD-46818.1 | UMdTsdT |
| AD-47355.1 | FOME |
| AD-47382.1 | DECAF |
| AD-45096.1end | Endolight |
| AD-45155.1 | Fluorolight |
| AD-46820.1 | UMdTsdT |
| AD-47358.1 | FOME |
| AD-47385.1 | DECAF |
| AD-45102.1end | Endolight |
| AD-45160.1 | Fluorolight |
| AD-46823.1 | UMdTsdT |
| AD-47335.1 | FOME |
| AD-47362.1 | DECAF |
| AD-45108.1end | Endolight |
| AD-45124.1 | Fluorolight |
| AD-46826.1 | UMdTsdT |
| AD-47339.1 | FOME |
| AD-47366.1 | DECAF |
| AD-45120.1end | Endolight |
| AD-45136.1 | Fluorolight |
| AD-46829.1 | UMdTsdT |
| AD-47347.1 | FOME |
| AD-47374.1 | DECAF |
| AD-45127.1end | Endolight |
| AD-45146.1 | Fluorolight |
| AD-46832.1 | UMdTsdT |
| AD-47353.1 | FOME |
| AD-47380.1 | DECAF |
| AD-45133.1end | Endolight |
| AD-45151.1 | Fluorolight |
| AD-46812.1 | UMdTsdT |
| AD-47356.1 | FOME |
| AD-47383.1 | DECAF |
| AD-45143.1end | Endolight |
| AD-45161.1 | Fluorolight |
| AD-46816.1 | UMdTsdT |
| AD-47336.1 | FOME |
| AD-47363.1 | DECAF |
| AD-45148.1end | Endolight |
| AD-45125.1 | Fluorolight |
| AD-46819.1 | UMdTsdT |
| AD-47340.1 | FOME |
| AD-47367.1 | DECAF |
| AD-45153.1end | Endolight |
| AD-45131.1 | Fluorolight |
| AD-46821.1 | UMdTsdT |
| AD-47344.1 | FOME |
| AD-47371.1 | DECAF |
| AD-45158.1end | Endolight |
| AD-45137.1 | Fluorolight |
| AD-46824.1 | UMdTsdT |
| AD-47348.1 | FOME |
| AD-47375.1 | DECAF |
| AD-45128.1end | Endolight |
| AD-45147.1 | Fluorolight |
| AD-46827.1 | UMdTsdT |
| AD-47354.1 | FOME |
| AD-45139.1end | Endolight |
| AD-45157.1 | Fluorolight |
| AD-46830.1 | UMdTsdT |
| AD-47360.1 | FOME |
| AD-47387.1 | DECAF |
| AD-45144.1end | Endolight |
| AD-45162.1 | Fluorolight |
| AD-46833.1 | UMdTsdT |
| AD-47337.1 | FOME |
| AD-47364.1 | DECAF |
| AD-45149.1end | Endolight |
| AD-45126.1 | Fluorolight |
| AD-46813.1 | UMdTsdT |
| AD-47341.1 | FOME |
| AD-47368.1 | DECAF |
| AD-45154.1end | Endolight |
| AD-45132.1 | Fluorolight |
| AD-46817.1 | UMdTsdT |
| AD-47345.1 | FOME |
| AD-47372.1 | DECAF |
| AD-45114.1end | Endolight |
| AD-45130.1 | Fluorolight |
| AD-47343.1 | FOME |
| AD-45141.1 | Fluorolight |
| AD-45121.1end | Endolight |
| AD-47350.1 | FOME |
| AD-45138.1end | Endolight |
| AD-47359.1 | Fluorolight |
| AD-47386.1 | DECAF |
| AD-45122.1end | Endolight |
| AD-47351.1 | FOMe |
| AD-47378.1 | DECAF |
| AD-45152.1 | Fluorolight |
| AD-47357.1 | FOMe |
| AD-47384.1 | DECAF |

TABLE 10
ApoC3 modified siRNA (third set) sequences

| | SEQ | | SEQ | |
|---|---|---|---|---|
| | ID | | ID | |
| DuplexName | NO: | Sense Sequence | NO: | Antisense sequence |
| AD-45101.1end | 526 | uGcAGccccGGGuAcuccudTsdT | 650 | AGGAGuACCCGGGGCUGcAdTsdT |
| AD-45159.1 | 527 | UfGCfAGCfCfCfCfGGGUfACfUfCfCfUfdTsdT | 651 | AGGAGUfACCCGGGGCUGCfAdTsdT |
| AD-46822.1 | 528 | UGCAGCCCCGGGUACUCCUdTsdT | 652 | AGGAGUACCCGGGGCUGCAdTsdT |
| AD-47334.1 | 529 | UfgCfaGfcCfcCfgGfgUfaCfuCfcUfdTsdT | 653 | aGfgAfgUfaCfcCfgGfgGfcUfgC |
| | | | | fadTsdT |
| AD-47361.1 | 530 | uGcAGccccGGGuAcuccudTsdT | 654 | AGGAGuACCCGGGGCugcadTsdT |
| AD-45107.1end | 531 | GcAGccccGGGuAcuccuudTsdT | 655 | AAGGAGuACCCGGGGCUGCdTsdT |
| AD-45123.1 | 532 | GCfAGCfCfCfCfGGGUfACfUfCfCfUfUfdTsdT | 656 | AAGGAGUfACCCGGGGCUGCdTsdT |
| AD-46825.1 | 533 | GCAGCCCCGGGUACUCCUUdTsdT | 657 | AAGGAGUACCCGGGGCUGCdTsdT |
| AD-47338.1 | 534 | GfcAfgCfcCfcGfgGfuAfcUfcCfuUfdTsdT | 658 | aAfgGfaGfuAfcCfcGfgGfgCfuG |
| | | | | fcdTsdT |
| AD-47365.1 | 535 | GcAGccccGGGuAcuccuudTsdT | 659 | AAGGAGuACCCGGGGCugcdTsdT |
| AD-45113.1end | 536 | cAAGAccGccAAGGAuGcAdTsdT | 660 | UGcAUCCUUGGCGGUCUUGdTsdT |
| AD-45129.1 | 537 | CfAAGACfCfGCfCfAAGGAUfGCfAdTsdT | 661 | UGCfAUCCUUGGCGGUCUUGdTsdT |
| AD-46828.1 | 538 | CAAGACCGCCAAGGAUGCAdTsdT | 662 | UGCAUCCUUGGCGGUCUUGdTsdT |
| AD-47342.1 | 539 | CfaAfgAfcCfgCfcAfaGfgAfuGfcAfdTsdT | 663 | uGfcAfuCfcUfuGfgCfgGfuCfuU |
| | | | | fgdTsdT |
| AD-47369.1 | 540 | cAAGAccGccAAGGAuGcAdTsdT | 664 | UGCAUCCuUGGCGGuCuugdTsdT |
| AD-45119.1end | 541 | uGGGuGAccGAuGGcuucAdTsdT | 665 | UGAAGCcAUCGGUcACCcAdTsdT |
| AD-45135.1 | 542 | UfGGGUfGACfCfGAUfGGCfUfUfCfAdTsdT | 666 | UGAAGCCfAUCGGUCfACCCfAdTsd |
| | | | | T |
| AD-46831.1 | 543 | UGGGUGACCGAUGGCUUCAdTsdT | 667 | UGAAGCCAUCGGUCACCCAdTsdT |
| AD-47346.1 | 544 | UfgGfgUfgAfcCfgAfuGfgCfuUfcAfdTsdT | 668 | uGfaAfgCfcAfuCfgGfuCfaCfcC |
| | | | | fadTsdT |
| AD-47373.1 | 545 | uGGGuGAccGAuGGcuucAdTsdT | 669 | UGAAGCCAUCGGuCACccadTsdT |
| AD-45078.1end | 546 | GGuGAccGAuGGcuucAGudTsdT | 670 | ACUGAAGCcAUCGGUcACCdTsdT |
| AD-45140.1 | 547 | GGUfGACfCfGAUfGGCfUfUfCfAGUfdTsdT | 671 | ACUGAAGCCfAUCGGUCfACCdTsdT |
| AD-46811.1 | 548 | GGUGACCGAUGGCUUCAGUdTsdT | 672 | ACUGAAGCCAUCGGUCACCdTsdT |
| AD-47349.1 | 549 | GfgUfgAfcCfgAfuGfgCfuUfcAfgUfdTsdT | 673 | aCfuGfaAfgCfcAfuCfgGfuCfaC |
| | | | | fcdTsdT |
| AD-47376.1 | 550 | GGuGAccGAuGGcuucAGudTsdT | 674 | ACuGAAGCCAuCGGuCaccdTsdT |
| AD-45084.1end | 551 | ccGAuGGcuucAGuucccudTsdT | 675 | AGGGAACUGAAGCcAUCGGdTsdT |
| AD-45145.1 | 552 | CfCfGAUfGGCfUfUfCfAGUfUfCfCfCfUfdTsdT | 676 | AGGGAACUGAAGCCfAUCGGdTsdT |
| AD-46815.1 | 553 | CCGAUGGCUUCAGUUCCCUdTsdT | 677 | AGGGAACUGAAGCCAUCGGdTsdT |
| AD-47352.1 | 554 | CfcGfaUfgGfcUfuCfaGfuUfcCfcUfdTsdT | 678 | aGfgGfaAfcUfgAfaGfcCfaUfcG |
| | | | | fgdTsdT |
| AD-47379.1 | 555 | ccGAuGGcuucAGuucccudTsdT | 679 | AGGGAACuGAAGCCAucggdTsdT |
| AD-45090.1end | 556 | GAuGGcuucAGuucccuGAdTsdT | 680 | UcAGGGAACUGAAGCcAUCdTsdT |
| AD-45150.1 | 557 | GAUfGGCfUfUfCfAGUfUfCfCfCfUfGAdTsdT | 681 | UCfAGGGAACUGAAGCCfAUCdTsdT |
| AD-46818.1 | 558 | GAUGGCUUCAGUUCCCUGAdTsdT | 682 | UCAGGGAACUGAAGCCAUCdTsdT |
| AD-47355.1 | 559 | GfaUfgGfcUfuCfaGfuUfcCfcUfgAfdTsdT | 683 | uCfaGfgGfaAfcUfgAfaGfcCfaU |
| | | | | fcdTsdT |
| AD-47382.1 | 560 | GAuGGcuucAGuucccuGAdTsdT | 684 | UCAGGGAACuGAAGCCaucdTsdT |
| AD-45096.1end | 561 | AuGGcuucAGuucccuGAAdTsdT | 685 | UUcAGGGAACUGAAGCcAUdTsdT |
| AD-45155.1 | 562 | AUfGGCfUfUfCfAGUfUfCfCfCfUfGAAdTsdT | 686 | UUCfAGGGAACUGAAGCCfAUdTsdT |
| AD-46820.1 | 563 | AUGGCUUCAGUUCCCUGAAdTsdT | 687 | UUCAGGGAACUGAAGCCAUdTsdT |
| AD-47358.1 | 564 | AfuGfgCfuUfcAfgUfuCfcCfuGfaAfdTsdT | 688 | uUfcAfgGfgAfaCfuGfaAfgCfcA |
| | | | | fudTsdT |
| AD-47385.1 | 565 | AuGGcuucAGuucccuGAAdTsdT | 689 | UUCAGGGAACuGAAGCcaudTsdT |
| AD-45102.1end | 566 | uGGcuucAGuucccuGAAAdTsdT | 690 | UUUcAGGGAACUGAAGCcAdTsdT |
| AD-45160.1 | 567 | UfGGCfUfUfCfAGUfUfCfCfCfUfGAAAdTsdT | 691 | UUUCfAGGGAACUGAAGCCfAdTsdT |
| AD-46823.1 | 568 | UGGCUUCAGUUCCCUGAAAdTsdT | 692 | UUUCAGGGAACUGAAGCCAdTsdT |
| AD-47335.1 | 569 | UfgGfcUfuCfaGfuUfcCfcUfgAfaAfdTsdT | 693 | uUfuCfaGfgGfaAfcUfgAfaGfcC |
| | | | | fadTsdT |
| AD-47362.1 | 570 | uGGcuucAGuucccuGAAAdTsdT | 694 | UuUCAGGGAACuGAAGccadTsdT |
| AD-45108.1end | 571 | GcuucAGuucccuGAAAGAdTsdT | 695 | UCUUUcAGGGAACUGAAGCdTsdT |
| AD-45124.1 | 572 | GCfUfUfCfAGUfUfCfCfCfUfGAAAGAdTsdT | 696 | UCUUUCfAGGGAACUGAAGCdTsdT |
| AD-46826.1 | 573 | GCUUCAGUUCCCUGAAAGAdTsdT | 697 | UCUUUCAGGGAACUGAAGCdTsdT |
| AD-47339.1 | 574 | GfcUfuCfaGfuUfcCfcUfgAfaAfgAfdTsdT | 698 | uCfuUfuCfaGfgGfaAfcUfgAfaG |
| | | | | fcdTsdT |
| AD-47366.1 | 575 | GcuucAGuucccuGAAAGAdTsdT | 699 | UCuUUCAGGGAACuGAagcdTsdT |
| AD-45120.1end | 576 | cuGAAAGAcuAcuGGAGcAdTsdT | 700 | UGCUCcAGuAGUCUUUcAGdTsdT |
| AD-45136.1 | 577 | CfUfGAAAGACfUfACfUfGGAGCfAdTsdT | 701 | UGCUCCfAGUfAGUCUUUCfAGdTsd |
| | | | | T |
| AD-46829.1 | 578 | CUGAAAGACUACUGGAGCAdTsdT | 702 | UGCUCCAGUAGUCUUUCAGdTsdT |
| AD-47347.1 | 579 | CfuGfaAfaGfaCfuAfcUfgGfaGfcAfdTsdT | 703 | uGfcUfcCfaGfuAfgUfcUfuUfcA |
| | | | | fgdTsdT |
| AD-47374.1 | 580 | cuGAAAGAcuAcuGGAGcAdTsdT | 704 | UGCUCCAGuAGuCuuucagdTsdT |
| AD-45127.1end | 581 | AGcAccGuuAAGGAcAAGudTsdT | 705 | ACUUGUCCUuAACGGUGCUdTsdT |
| AD-45146.1 | 582 | AGCfACfCfGUfUfAAGGACfAAGUfdTsdT | 706 | ACUUGUCCUUfAACGGUGCUdTsdT |
| AD-46832.1 | 583 | AGCACCGUUAAGGACAAGUdTsdT | 707 | ACUUGUCCUUAACGGUGCUdTsdT |
| AD-47353.1 | 584 | AfgCfaCfcGfuUfaAfgGfaCfaAfgUfdTsdT | 708 | aCfuUfgUfcCfuUfaAfcGfgUfgC |
| | | | | fudTsdT |
| AD-47380.1 | 585 | AGcAccGuuAAGGAcAAGudTsdT | 709 | ACuUGUCCuUAACGGugcudTsdT |
| AD-45133.1end | 586 | GcAccGuuAAGGAcAAGuudTsdT | 710 | AACUUGUCCUuAACGGUGCdTsdT |
| AD-45151.1 | 587 | GCfACfCfGUfUfAAGGACfAAGUfUfdTsdT | 711 | AACUUGUCCUUfAACGGUGCdTsdT |
| AD-46812.1 | 588 | GCACCGUUAAGGACAAGUUdTsdT | 712 | AACUUGUCCUUAACGGUGCdTsdT |
| AD-47356.1 | 589 | GfcAfcCfgUfuAfaGfgAfcAfaGfuUfdTsdT | 713 | aAfcUfuGfuCfcUfuAfaCfgGfuG |
| | | | | fcdTsdT |
| AD-47383.1 | 590 | GcAccGuuAAGGAcAAGuudTsdT | 714 | AACuUGUCCuuAACGGugcdTsdT |
| AD-45143.1end | 591 | GcuGccuGAGAccucAAuAdTsdT | 715 | uAUUGAGGUCUcAGGcAGCdTsdT |
| AD-45161.1 | 592 | GCfUfGCfCfUfGAGACfCfUfCfAAUfAdTsdT | 716 | UfABUGAGGUCUCfAGGCfAGCdTsd |
| | | | | T |
| AD-46816.1 | 593 | GCUGCCUGAGACCUCAAUAdTsdT | 717 | UAUUGAGGUCUCAGGCAGCdTsdT |
| AD-47336.1 | 594 | GfcUfgCfcUfgAfgAfcCfuCfaAfuAfdTsdT | 718 | uAfuUfgAfgGfuCfuCfaGfgCfaG |
| | | | | fcdTsdT |
| AD-47363.1 | 595 | GcuGccuGAGAccucAAuAdTsdT | 719 | UAuUGAGGUCuCAGGCagcdTsdT |
| AD-45148.1end | 596 | cuGAGAccucAAuAccccAdTsdT | 720 | UGGGGuAUUGAGGUCUcAGdTsdT |
| AD-45125.1 | 597 | CfUfGAGACfCfUfCfAAUfACfCfCfCfAdTsdT | 721 | UGGGGUfABUGAGGUCUCfAGdTsdT |
| AD-46819.1 | 598 | CUGAGACCUCAAUACCCCAdTsdT | 722 | UGGGGUAUUGAGGUCUCAGdTsdT |
| AD-47340.1 | 599 | CfuGfaGfaCfcUfcAfaUfaCfcCfcAfdTsdT | 723 | uGfgGfgUfaUfuGfaGfgUfcUfcA |
| | | | | fgdTsdT |
| AD-47367.1 | 600 | cuGAGAccucAAuAccccAdTsdT | 724 | UGGGGuAuUGAGGuCucagdTsdT |
| AD-45153.1end | 601 | uGAGAccucAAuAccccAAdTsdT | 725 | UUGGGGuAUUGAGGUCUcAdTsdT |
| AD-45131.1 | 602 | UfGAGACfCfUfCfAAUfACfCfCfCfAAdTsdT | 726 | UUGGGGUfABUGAGGUCUCfAdTsdT |
| AD-46821.1 | 603 | UGAGACCUCAAUACCCCAAdTsdT | 727 | UUGGGGUAUUGAGGUCUCAdTsdT |
| AD-47344.1 | 604 | UfgAfgAfcCfuCfaAfuAfcCfcCfaAfdTsdT | 728 | uUfgGfgGfuAfuUfgAfgGfuCfuC |
| | | | | fadTsdT |
| AD-47371.1 | 605 | uGAGAccucAAuAccccAAdTsdT | 729 | UuGGGGuAuUGAGGuCucadTsdT |
| AD-45158.1end | 606 | ccucAAuAccccAAGuccAdTsdT | 730 | UGGACUUGGGGuAUUGAGGdTsdT |
| AD-45137.1 | 607 | CfCfUfCfAAUfACfCfCfCfAAGUfCfCfAdTsdT | 731 | UGGACUUGGGGUfABUGAGGdTsdT |
| AD-46824.1 | 608 | CCUCAAUACCCCAAGUCCAdTsdT | 732 | UGGACUUGGGGUAUUGAGGdTsdT |
| AD-47348.1 | 609 | CfcUfcAfaUfaCfcCfcAfaGfuCfcAfdTsdT | 733 | uGfgAfcUfuGfgGfgUfaUfuGfaG |
| | | | | fgdTsdT |
| AD-47375.1 | 610 | ccucAAuAccccAAGuccAdTsdT | 734 | UGGACuUGGGGuAuuGaggdTsdT |
| AD-45128.1end | 611 | GcuGccccuGuAGGuuGcudTsdT | 735 | AGcAACCuAcAGGGGcAGCdTsdT |
| AD-45147.1 | 612 | GCfUfGCfCfCfCfUfGUfAGGUfUfGCfUfdTsdT | 736 | AGCfAACCUfACfAGGGGCfAGCdTs |
| | | | | dT |
| AD-46827.1 | 613 | GCUGCCCCUGUAGGUUGCUdTsdT | 737 | AGCAACCUACAGGGGCAGCdTsdT |
| AD-47354.1 | 614 | GfcUfgCfcCfcUfgUfaGfgUfuGfcUfdTsdT | 738 | aGfcAfaCfcUfaCfaGfgGfgCfaG |
| | | | | fcdTsdT |
| AD-45139.1end | 615 | AGGuuGcuuAAAAGGGAcAdTsdT | 739 | UGUCCCUUUuAAGcAACCUdTsdT |
| AD-45157.1 | 616 | AGGUfUfGCfUfUfAAAAGGGACfAdTsdT | 740 | UGUCCCUUUUfAAGCfAACCUdTsdT |
| AD-46830.1 | 617 | AGGUUGCUUAAAAGGGACAdTsdT | 741 | UGUCCCUUUUAAGCAACCUdTsdT |
| AD-47360.1 | 618 | AfgGfuUfgCfuUfaAfaAfgGfgAfcAfdTsdT | 742 | uGfuCfcCfuUfuUfaAfgCfaAfcC |
| | | | | fudTsdT |
| AD-47387.1 | 619 | AGGuuGcuuAAAAGGGAcAdTsdT | 743 | UGUCCCuUuUAAGCAAccudTsdT |
| AD-45144.1end | 620 | uGcuuAAAAGGGAcAGuAudTsdT | 744 | AuACUGUCCCUUUuAAGcAdTsdT |
| AD-45162.1 | 621 | UfGCfUfUfAAAAGGGACfAGUfAUfdTsdT | 745 | AUfACUGUCCCUUUUfAAGCfAdTsd |
| | | | | T |
| AD-46833.1 | 622 | UGCUUAAAAGGGACAGUAUdTsdT | 746 | AUACUGUCCCUUUUAAGCAdTsdT |
| AD-47337.1 | 623 | UfgCfuUfaAfaAfgGfgAfcAfgUfaUfdTsdT | 747 | aUfaCfuGfuCfcCfuUfuUfaAfgC |
| | | | | fadTsdT |
| AD-47364.1 | 624 | uGcuuAAAAGGGAcAGuAudTsdT | 748 | AuACuGUCCCuuuuAAgcadTsdT |
| AD-45149.1end | 625 | GcuuAAAAGGGAcAGuAuudTsdT | 749 | AAuACUGUCCCUUUuAAGCdTsdT |
| AD-45126.1 | 626 | GCfUfUfAAAAGGGACfAGUfAUfUfdTsdT | 750 | AAUfACUGUCCCUUUUfAAGCdTsdT |
| AD-46813.1 | 627 | GCUUAAAAGGGACAGUAUUdTsdT | 751 | AAUACUGUCCCUUUUAAGCdTsdT |
| AD-47341.1 | 628 | GfcUfuAfaAfaGfgGfaCfaGfuAfuUfdTsdT | 752 | aAfuAfcUfgUfcCfcUfuUfuAfaG |
| | | | | fcdTsdT |
| AD-47368.1 | 629 | GcuuAAAAGGGAcAGuAuudTsdT | 753 | AAuACuGUCCCuuuuAagcdTsdT |
| AD-45154.1end | 630 | cuGGAcAAGAAGcuGcuAudTsdT | 754 | AuAGcAGCUUCUUGUCcAGdTsdT |
| AD-45132.1 | 631 | CfUfGGACfAAGAAGCfUfGCfUfAUfdTsdT | 755 | AUfAGCfAGCUUCUUGUCCfAGdTsd |
| | | | | T |
| AD-46817.1 | 632 | CUGGACAAGAAGCUGCUAUdTsdT | 756 | AUAGCAGCUUCUUGUCCAGdTsdT |
| AD-47345.1 | 633 | CfuGfgAfcAfaGfaAfgCfuGfcUfaUfdTsdT | 757 | aUfaGfcAfgCfuUfcUfuGfuCfcA |
| | | | | fgdTsdT |
| AD-47372.1 | 634 | cuGGAcAAGAAGcuGcuAudTsdT | 758 | AuAGCAGCuUCuuGuCcagdTsdT |
| AD-45114.1end | 635 | ucccuGAAAGAcuAcuGGAdTsdT | 759 | UCcAGuAGUCUUUcAGGGAdTsdT |
| AD-45130.1 | 636 | UfCfCfCfUfGAAAGACfUfACfUfGGAdTsdT | 760 | UCCfAGUfAGUCUUUCfAGGGAdTsd |
| | | | | T |
| AD-47343.1 | 637 | UfcCfcUfgAfaAfgAfcUfaCfuGfgAfdTsdT | 761 | uCfcAfgUfaGfuCfuUfuCfaGfgG |
| | | | | fadTsdT |
| AD-45141.1 | 638 | AGACfUfACfUfGGAGCfACfCfGUfUfdTsdT | 762 | AACGGUGCUCCfAGUfAGUCUdTsdT |
| AD-45121.1end | 639 | AGAcuAcuGGAGcAccGuudTsdT | 763 | AACGGUGCUCcAGuAGUCUdTsdT |
| AD-47350.1 | 640 | AfgAfcUfaCfuGfgAfgCfaCfcGfuUfdTsdT | 764 | aAfcGfgUfgCfuCfcAfgUfaGfuC |
| | | | | fudTsdT |
| AD-45138.1end | 641 | GGcuGccuGAGAccucAAudTsdT | 765 | AUUGAGGUCUcAGGcAGCCdTsdT |
| AD-47359.1 | 642 | GfgCfuGfcCfuGfaGfaCfcUfcAfaUfdTsdT | 766 | aUfuGfaGfgUfcUfcAfgGfcAfgC |
| | | | | fcdTsdT |
| AD-47386.1 | 643 | GGcuGccuGAGAccucAAudTsdT | 767 | AuUGAGGUCuCAGGCAgccdTsdT |
| AD-45122.1end | 644 | cAGGGcuGccccuGuAGGudTsdT | 768 | ACCuAcAGGGGcAGCCCUGdTsdT |
| AD-47351.1 | 645 | CfaGfgGfcUfgCfcCfcUfgUfaGfgUfdTsdT | 769 | aCfcUfaCfaGfgGfgCfaGfcCfcU |
| | | | | fgdTsdT |
| AD-45152.1 | 647 | CfCfCfUfGUfAGGUfUfGCfUfUfAAAAdTsdT | 771 | UUUUfAAGCfAACCUfACfAGGGdTs |
| | | | | dT |
| AD-47357.1 | 648 | CfcCfuGfuAfgGfuUfgCfuUfaAfaAfdTsdT | 772 | uUfuUfaAfgCfaAfcCfuAfcAfgG |
| | | | | fgdTsdT |
| AD-47384.1 | 649 | cccuGuAGGuuGcuuAAAAdTsdT | 773 | UuUuAAGCAACCuACAgggdTsdT |

TABLE 11
ApoC3 modified siRNA (third set) Activity in cells

| Duplex Name | 10 nM | 0.1 nM | 10 nM SD | 0.1 nM SD |
|---|---|---|---|---|
| AD-45101.1end | 1.01 | 1.30 | 0.22 | 0.42 |
| AD-45159.1 | 0.12 | 0.53 | 0.01 | 0.03 |
| AD-46822.1 | 0.15 | 0.31 | 0.00 | 0.00 |
| AD-47334.1 | 0.41 | 0.82 | 0.12 | 0.10 |
| AD-47361.1 | 0.89 | 1.00 | 0.19 | 0.03 |
| AD-45107.1end | 0.87 | 1.06 | 0.02 | 0.08 |
| AD-45123.1 | 0.15 | 0.41 | 0.02 | 0.02 |
| AD-46825.1 | 0.13 | 0.26 | 0.01 | 0.01 |
| AD-47338.1 | 0.38 | 0.73 | 0.12 | 0.12 |
| AD-47365.1 | 0.74 | 0.93 | 0.01 | 0.07 |
| AD-45113.1end | 0.97 | 1.04 | 0.20 | 0.01 |
| AD-45129.1 | 0.20 | 0.47 | 0.07 | 0.06 |
| AD-46828.1 | 0.15 | 0.57 | 0.00 | 0.04 |
| AD-47342.1 | 0.38 | 0.91 | 0.26 | 0.06 |
| AD-47369.1 | 1.10 | 1.26 | 0.18 | 0.29 |
| AD-45119.1end | 0.91 | 0.87 | 0.18 | 0.05 |
| AD-45135.1 | 0.02 | 0.08 | 0.01 | 0.02 |
| AD-46831.1 | 0.01 | 0.37 | 0.00 | 0.05 |
| AD-47346.1 | 0.57 | 0.95 | 0.17 | 0.08 |
| AD-47373.1 | 0.80 | 1.06 | 0.06 | 0.15 |
| AD-45078.1end | 0.38 | 0.78 | 0.08 | 0.05 |
| AD-45140.1 | 0.07 | 0.33 | 0.02 | 0.06 |
| AD-46811.1 | 0.03 | 0.31 | 0.01 | 0.04 |
| AD-47349.1 | 0.03 | 0.29 | 0.02 | 0.18 |
| AD-47376.1 | 0.38 | 0.95 | 0.15 | 0.01 |
| AD-45084.1end | 0.60 | 0.92 | 0.03 | 0.06 |
| AD-45145.1 | 0.04 | 0.13 | 0.01 | 0.02 |
| AD-46815.1 | 0.06 | 0.37 | 0.00 | 0.00 |
| AD-47352.1 | 0.04 | 0.35 | 0.04 | 0.23 |
| AD-47379.1 | 0.81 | 1.03 | 0.12 | 0.05 |
| AD-45090.1end | 0.97 | 0.86 | 0.06 | 0.05 |
| AD-45150.1 | 0.09 | 0.28 | 0.01 | 0.05 |
| AD-46818.1 | 0.03 | 0.26 | 0.00 | 0.03 |
| AD-47355.1 | 0.23 | 0.60 | 0.14 | 0.08 |
| AD-47382.1 | 0.40 | 0.81 | 0.11 | 0.09 |
| AD-45096.1end | 0.47 | 0.82 | 0.07 | 0.26 |
| AD-45155.1 | 0.60 | 0.68 | 0.11 | 0.07 |
| AD-46820.1 | 0.03 | 0.38 | 0.00 | 0.02 |
| AD-47358.1 | 0.05 | 0.45 | 0.03 | 0.31 |
| AD-47385.1 | 0.53 | 0.84 | 0.12 | 0.19 |
| AD-45102.1end | 0.05 | 0.22 | 0.03 | 0.03 |
| AD-45160.1 | 0.03 | 0.11 | 0.00 | 0.01 |
| AD-46823.1 | 0.02 | 0.22 | 0.00 | 0.04 |
| AD-47335.1 | 0.15 | 0.70 | 0.05 | 0.00 |
| AD-47362.1 | 0.66 | 1.07 | 0.01 | 0.09 |
| AD-45108.1end | 0.02 | 0.12 | 0.00 | 0.01 |
| AD-45124.1 | 0.03 | 0.07 | 0.01 | 0.01 |
| AD-46826.1 | 0.02 | 0.18 | 0.00 | 0.02 |
| AD-47339.1 | 0.19 | 0.62 | 0.12 | 0.04 |
| AD-47366.1 | 0.60 | 0.82 | 0.02 | 0.09 |
| AD-45120.1end | 0.03 | 0.08 | 0.01 | 0.03 |
| AD-45136.1 | 0.04 | 0.10 | 0.00 | 0.01 |
| AD-46829.1 | 0.02 | 0.22 | 0.01 | 0.01 |
| AD-47347.1 | 0.16 | 0.66 | 0.14 | 0.11 |
| AD-47374.1 | 0.90 | 1.15 | 0.03 | 0.03 |
| AD-45127.1end | 0.58 | 0.75 | 0.02 | 0.05 |
| AD-45146.1 | 0.06 | 0.21 | 0.00 | 0.00 |
| AD-46832.1 | 0.09 | 0.56 | 0.02 | 0.01 |
| AD-47353.1 | 0.21 | 0.65 | 0.04 | 0.02 |
| AD-47380.1 | 0.66 | 1.02 | 0.07 | 0.02 |
| AD-45133.1end | 0.03 | 0.10 | 0.01 | 0.04 |
| AD-45151.1 | 0.03 | 0.05 | 0.01 | 0.00 |
| AD-46812.1 | 0.01 | 0.10 | 0.00 | 0.00 |
| AD-47356.1 | 0.02 | 0.13 | 0.01 | 0.13 |
| AD-47383.1 | 0.03 | 0.21 | 0.02 | 0.10 |
| AD-45143.1end | 0.73 | 0.94 | 0.06 | 0.09 |
| AD-45161.1 | 0.59 | 0.54 | 0.05 | 0.06 |
| AD-46816.1 | 0.04 | 0.53 | 0.00 | 0.01 |
| AD-47336.1 | 0.10 | 0.37 | 0.06 | 0.14 |
| AD-47363.1 | 0.54 | 0.88 | 0.03 | 0.14 |
| AD-45148.1end | 0.80 | 0.94 | 0.06 | 0.10 |
| AD-45125.1 | 0.16 | 0.55 | 0.02 | 0.01 |
| AD-46819.1 | 0.06 | 0.49 | 0.01 | 0.02 |
| AD-47340.1 | 0.20 | 0.72 | 0.18 | 0.21 |
| AD-47367.1 | 0.99 | 1.10 | 0.17 | 0.07 |
| AD-45153.1end | 0.92 | 1.02 | 0.06 | 0.05 |
| AD-45131.1 | 0.24 | 0.64 | 0.04 | 0.00 |
| AD-46821.1 | 0.19 | 0.67 | 0.01 | 0.01 |
| AD-47344.1 | 0.48 | 0.90 | 0.16 | 0.06 |
| AD-47371.1 | 1.01 | 0.92 | 0.14 | 0.12 |
| AD-45158.1end | 0.37 | 0.78 | 0.04 | 0.00 |
| AD-45137.1 | 0.02 | 0.19 | 0.00 | 0.06 |
| AD-46824.1 | 0.02 | 0.21 | 0.00 | 0.02 |
| AD-47348.1 | 0.19 | 0.66 | 0.20 | 0.24 |
| AD-47375.1 | 0.83 | 0.94 | 0.06 | 0.00 |
| AD-45128.1end | 0.97 | 0.96 | 0.18 | 0.08 |
| AD-45147.1 | 0.20 | 0.49 | 0.01 | 0.16 |
| AD-46827.1 | 0.05 | 0.54 | 0.01 | 0.06 |
| AD-47354.1 | 0.23 | 0.79 | 0.15 | 0.19 |
| AD-45139.1end | 1.19 | 1.13 | 0.46 | 0.26 |
| AD-45157.1 | 0.13 | 0.29 | 0.02 | 0.11 |
| AD-46830.1 | 0.64 | 1.01 | 0.03 | 0.00 |
| AD-47360.1 | 0.76 | 1.22 | 0.09 | 0.22 |
| AD-47387.1 | 0.90 | 0.84 | 0.04 | 0.09 |
| AD-45144.1end | 0.45 | 0.95 | 0.15 | 0.20 |
| AD-45162.1 | 0.02 | 0.06 | 0.00 | 0.03 |
| AD-46833.1 | 0.06 | 0.46 | 0.01 | 0.05 |
| AD-47337.1 | 0.05 | 0.23 | 0.03 | 0.16 |
| AD-47364.1 | 0.52 | 0.79 | 0.10 | 0.02 |
| AD-45149.1end | 0.03 | 0.07 | 0.00 | 0.03 |
| AD-45126.1 | 0.03 | 0.08 | 0.02 | 0.02 |
| AD-46813.1 | 0.02 | 0.27 | 0.00 | 0.01 |
| AD-47341.1 | 0.04 | 0.17 | 0.02 | 0.11 |
| AD-47368.1 | 0.46 | 0.56 | 0.02 | 0.13 |
| AD-45154.1end | 0.14 | 0.29 | 0.00 | 0.04 |
| AD-45132.1 | 0.30 | 0.52 | 0.04 | 0.05 |
| AD-46817.1 | 0.10 | 0.29 | 0.01 | 0.04 |
| AD-47345.1 | 0.27 | 0.58 | 0.17 | 0.19 |
| AD-47372.1 | 0.34 | 0.44 | 0.17 | 0.04 |
| AD-45114.1end | 0.37 | 0.77 | 0.07 | 0.15 |
| AD-45130.1 | 0.05 | 0.12 | 0.03 | 0.00 |
| AD-47343.1 | 0.21 | 0.66 | 0.15 | 0.18 |
| AD-45141.1 | 0.03 | 0.07 | 0.00 | 0.00 |
| AD-45121.1end | 0.93 | 0.94 | 0.05 | 0.02 |
| AD-47350.1 | 0.12 | 0.58 | 0.05 | 0.31 |
| AD-45138.1end | 0.86 | 1.04 | 0.06 | 0.33 |
| AD-47359.1 | 0.82 | 1.04 | 0.10 | 0.18 |
| AD-47386.1 | 0.88 | 1.28 | 0.15 | 0.21 |
| AD-45122.1end | 0.92 | 0.97 | 0.02 | 0.11 |
| AD-47351.1 | 0.48 | 1.01 | 0.18 | 0.14 |
| AD-47378.1 | 1.10 | 1.08 | 0.09 | 0.03 |
| AD-45152.1 | 0.04 | 0.13 | 0.01 | 0.02 |
| AD-47357.1 | 0.09 | 0.28 | 0.02 | 0.25 |
| AD-47384.1 | 0.09 | 0.35 | 0.01 | 0.17 |

| NCBI Reference Sequence: NM_000040.1, Homo sapiens Apolipoprotein | |
|---|---|
| C-III(APOC3), mRNA | |
| SEQ ID NO: 1 | |
| 1 | tgctcagttc atccctagag gcagctgctc caggaacaga ggtgccatgc agccccgggt |
| 61 | actccttgtt gttgccctcc tggcgctcct ggcctctgcc cgagcttcag aggccgagga |
| 121 | tgcctccctt ctcagcttca tgcagggtta catgaagcac gccaccaaga ccgccaagga |
| 181 | tgcactgagc agcgtgcagg agtcccaggt ggcccagcag gccaggggct gggtgaccga |
| 241 | tggcttcagt tccctgaaag actactggag caccgttaag gacaagttct ctgagttctg |
| 301 | ggatttggac cctgaggtca gaccaacttc agccgtggct gcctgagacc tcaatacccc |
| 361 | aagtccacct gcctatccat cctgcgagct ccttgggtcc tgcaatctcc agggctgccc |
| 421 | ctgtaggttg cttaaaaggg acagtattct cagtgctctc ctaccccacc tcatgcctgg |
| 481 | cccccctcca ggcatgctgg cctcccaata aagctggaca agaagctgct atg |

1. A double-stranded ribonucleic acid (dsRNA) for inhibiting expression of an APOC3 gene,
wherein the dsRNA comprises a sense strand and an antisense strand forming a double stranded region, wherein the sense strand and the antisense strand are each 30 nucleotides or less in length, and wherein the nucleotide sequence of the antisense strand comprises at least 15 contiguous nucleotides of any one of the antisense strand nucleotide sequences in any one of Tables 1, 2, 6, 7, or 10.

2. (canceled)

3. The dsRNA of claim 1, wherein the nucleotide sequence of the sense strand is selected from any one of the sense strand nucleotide sequences in any one of Tables 1, 2, 6, 7, or 10, and the nucleotide sequence of the antisense strand is selected from any one of the antisense strand nucleotide sequences in any one of Tables 1, 2, 6, 7, or 10.

4. The dsRNA of claim 1, wherein at least one nucleotide of the dsRNA is a modified nucleotide.

5. The dsRNA of claim 4, wherein the modified nucleotide is selected from the group consisting of: a 2′-O-methyl modified nucleotide, a nucleotide comprising a 5′-phosphorothioate group, and a terminal nucleotide linked to a cholesteryl derivative or dodecanoic acid bisdecylamide group.

6. The dsRNA of claim 4, wherein the modified nucleotide is selected from the group consisting of: a 2′-deoxy-2′-fluoro modified nucleotide, a 2′-deoxy-modified nucleotide, a locked nucleotide, an abasic nucleotide, 2′-amino-modified nucleotide, 2′-alkyl-modified nucleotide, morpholino nucleotide, a phosphoramidate, and a non-natural base comprising nucleotide.

7. The dsRNA of claim 1, wherein at least one strand comprises a 3′ overhang of at least 1 nucleotide.

8. The dsRNA of claim 1, wherein each strand comprises a 3′ overhang of at 2 nucleotides.

9. The dsRNA of claim 1, further comprising a ligand.

10. The dsRNA of claim 9, wherein the ligand is conjugated to the 3′ end of the sense strand of the dsRNA.

11. The dsRNA of claim 9 wherein the ligand is an N-Acetyl-Galactosamine derivative.

12. An isolated cell comprising the dsRNA of claim 1.

13. A vector encoding at least one strand of the dsRNA of claim 1.

14. An isolated cell comprising the vector of claim 13.

15. A pharmaceutical composition for inhibiting expression of an APOC3 gene comprising the dsRNA of claim 1.

16. The pharmaceutical composition of claim 15, comprising a lipid formulation.

17. The pharmaceutical composition of claim 15, comprising a lipid formulation comprising MC3.

18. A method of inhibiting APOC3 expression in a cell, the method comprising:
(a) contacting the cell with the dsRNA of claim 1; and
(b) maintaining the cell produced in step (a) for a time sufficient to obtain degradation of the mRNA transcript of an APOC3 gene, thereby inhibiting expression of the APOC3 gene in the cell.

19. The method of claim 18, wherein the APOC3 expression is inhibited by at least 30%.

20. A method of treating a disorder mediated by APOC3 expression comprising administering to a human in need of such treatment a therapeutically effective amount of the APOC3 dsRNA of claim 1.

21.-24. (canceled)
